(19) United States
(12) Patent Application Publication
(10) Pub. No.: US 2023/0255933 A1
(43) Pub. Date: Aug. 17, 2023

(54) ANTIVIRAL USE OF FABP4 MODULATING COMPOUNDS

(71) Applicant: CRESCENTA BIOSCIENCES, Union, NJ (US)

(72) Inventors: Emre KOYUNCU, Irvine, CA (US); Hahn KIM, Princeton, NJ (US); Gokhan HOTAMISLIGIL, Weston, MA (US)

(73) Assignee: CRESCENTA BIOSCIENCES, Union, NJ (US)

(21) Appl. No.: 18/014,981

(22) PCT Filed: Jul. 6, 2021
(86) PCT No.: PCT/US21/40588

§ 371 Date: Jan. 6, 2023

Related U.S. Application Data
(60) Provisional application No. 63/048,609, filed on Jul. 6, 2020.

(51) Int. Cl. A61K 31/403; A61P 31/14

(52) U.S. Cl. CPC A61K 31/403; A61P 31/14; C07B 2200/07

(57) ABSTRACT

A method for treating a viral disease in a subject comprising administering to said subject a therapeutically effective dose of one or more compounds that bind to fatty acid binding protein FABP4, with compounds described in the specification some having the general formula, Formula I-Formula XII, where W₁-W₄, Z₁-Z₄, Z₁-Z₅, X, Y, n, and R₁-R₈ are as defined in the claims and description of embodiments. In examples, the ring Z contains Z₁-Z₄. In other examples, the ring Z contains Z₁-Z₅.

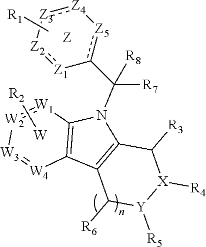

### CROSS-REFERENCE TO RELATED APPLICATIONS SECTION

This application is a U.S. national phase application of a PCT Application No. PCT/US2021/040588 filed on Jul. 6, 2021, which claims priority to U.S. Provisional Patent Application Ser. 63/048,609 filed on Jul. 6, 2020, the entire contents of which are hereby incorporated by reference in their entirety.

### FIELD OF THE EMBODIMENTS

The field of the embodiments of the present invention relate to antiviral therapies for treatment or prophylaxis of diseases caused by coronavirus infections. Compounds in this invention particularly interact with fatty acid binding protein 4 (FABP4) and inhibit the growth of coronaviruses in cells.

### BACKGROUND OF THE EMBODIMENTS

FABPs are a family of proteins that reversibly bind free fatty acids and other lipid molecules and facilitate their transport in cells. To date, nine different FABP isoforms have been identified in mammals. FABP isoforms display differential expression patterns in different tissues. Fatty acid binding protein 4 (FABP4), also often referred to as aP2 in literature, is primarily expressed in adipocytes and macrophages, and mediates key metabolic and inflammatory pathways in these cells such as lipid storage and degradation, signaling and eicosanoid production. In addition, FABP4 is also secreted to plasma and has been proposed to act as an adipose-derived factor regulating systemic glucose homeostasis.

The present invention relates to the pharmaceutical industry and medicines and presents a new therapy for the treatment and prevention of acute viral infections, particularly acute respiratory viral infections and more particularly to therapy for coronavirus and severe acute respiratory syndrome (SARS) diseases, comprising the administration of a therapeutically effective dose of a compound described herein to a subject in need thereof.

Acute respiratory viral infections are a group of infectious diseases of viral etiology and characterized by damage to the respiratory tract and exhibiting common clinical symptoms. The successes achieved by medicine in the field of infectious pathology have contributed to the development and improvement of methods for the prevention and treatment of diseases, but acute respiratory viral infections continue to be a serious public health problem due to extremely high rates of incidence and the occurrence of epidemics and pandemics.

Acute respiratory viral infections are polyetiological. Currently, more than 200 relevant pathogens are known with rhinoviruses, coronaviruses, and influenza viruses frequently seen causing a high outbreak in the autumn and winter. Other viruses causing acute respiratory viral infections at relatively higher rates include respiratory syncytial viruses (RSVs), parainfluenza, adenoviruses, Human metapneumovirus (HMPV), Human Bocavirus (HBoV) along with unidentified viruses responsible for the remainder of acute infections of the respiratory tract of viral and unspecified etiology.

Of recent concern, SARS, a form of acute respiratory viral infection, presents as one of the leading severe diseases active in children and adults, resulting in temporary disability and significant mortality. The high incidence of SARS is associated with significant economic losses due to indirect costs associated with disability and compounded by societal and economic damage from governmentally imposed lockdowns instituted to prevent viral spread. Most seasonal morbidity is affected by children, the elderly, people with concomitant diseases (various immunodeficiencies, diseases of the lungs, cardiovascular system, liver, kidneys, diabetes, etc.).

Acute respiratory diseases of coronavirus etiology can be severe and cause extensive damage to lung tissue as well as other organs. Common human coronaviruses cause varying levels of acute respiratory infections. Viruses HCoV-OC43, and HCoV-HKU1, HCoV-229E, and HCoV-NL63 cause common colds and self-limiting upper respiratory infections in immunocompetent individuals. In immunocompromised subjects and the elderly, lower respiratory tract infections can occur. Other coronaviruses can cause more severe form of acute respiratory disease and include SARS-CoV, SARS-CoV-2, and MERS-CoV. These coronaviruses can cause epidemics and pandemics with variable clinical severity featuring respiratory and extra-respiratory manifestations.

The COVID-19 pandemic caused by SARS-CoV-2 dramatically demonstrated that there is a great unmet need for effective therapeutic agents that can broadly treat or prevent coronavirus infections. This highlighted the importance of host factors, independent of the viral proteins, that are intimately related to the virus life cycle and also the disease severity and mortality associated with viral infections.

The invention and embodiments thereof disclosed herein describes a novel class of antiviral agents that interact with fatty acid binding protein 4 (FABP4) and inhibit viral replication in human cells. FABP4 is a key mediator of fatty acid metabolism and inflammation, two pathways that have been implicated in the life cycle of many viral pathogens. Furthermore, FABP4 has been particularly associated with the development of a number of metabolic conditions such as diabetes, cardiovascular disease, and airway inflammation that are known to confer susceptibility to coronavirus, influenza virus and certain other virus infections. In view of the present discovery that compounds that target FABP4 also inhibit viral replication in cells, the compounds described in this invention present a universally applicable treatment for disease caused by known viruses and variants that emerge in future.

### SUMMARY OF THE EMBODIMENTS

The present inventors have found that compounds that interact with a host protein FABP4 are potent and sustained inhibitors of viral replication in a host. The inventors have found that compounds that target FABP4 also inhibit viral replication and dramatically reduce virus production and the spread of infection. The inventors further found that certain compounds described herein are particularly suited to interact with FABP4 and protect against viruses causing acute respiratory viral infections. The inventors thus recognized that such compounds have applications in treating and/or preventing viral disease in a subject. The compounds can be advantageously provided in the form of an aerosol formulation. The compounds can advantageously be used in the form of a pharmaceutical composition comprising a pharmaceutically acceptable carrier or diluent. The compounds can also be advantageously used in the form of a combination comprising an additional antiviral agent. Such combination therapies have particular relevance in the prevention or treatment of viral infection caused by highly infectious viral strains such as epidemic or pandemic coronavirus.

The invention is a method for treating a viral disease in a subject comprising administering to said subject a therapeutically effective dose of a compound suited to interact with FABP4.

In further embodiments, the invention is a method for treating a viral disease in a subject comprising administering to said subject a therapeutically effective dose of one or more of the compounds described in Formula (I):

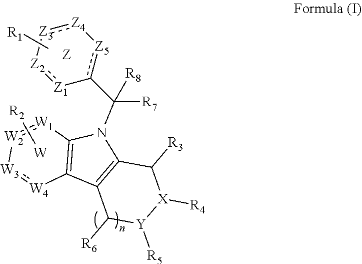

Wherein:

W₁₋₄ and Z₁-Z₅ are each independently —C, —CH, CH₂, O, S, or N;

X is independently CH₂, N or CHR₄;

Y is independently CH₂, or CHR₅;

n is a number between 0 and 3;

One or more R₁'s on the ring Z are independently selected from the group consisting of: CN, OH, COOH, OCH₃, CF₃, CONH₂, B(OH)₂, B(OR)₂, an acid isostere, a substituted amine, ethers, and a halogen, substituted or unsubstituted alkyl, substituted or unsubstituted cyclic or heterocyclic, substituted or unsubstituted cycloaryl or cychloheteroaryl, wherein the substituted cycloaryl or cychloheteroaryl may be substituted with hydrogen, CN, OH, COOH, OCH₃, CF₃, CONH₂, B(OH)₂, B(OR)₂, an acid isostere, a substituted amine, ethers, and a halogen, substituted or unsubstituted alkyl, substituted or unsubstituted cyclic or heterocyclic, substituted or unsubstituted cycloaryl or cychloheteroaryl, and SO₂NH₂;

One or more R₂'s on the ring W are independently selected from the group consisting of: CN, OH, CHF2, CH2F, CF₃, COOH, CONH₂, B(OH)₂, B(OR)₂, an acid isostere, a halogen, and a bicyclic heteroaryl;

R₇ is hydrogen or CN, COOH, CONH₂, B(OH)₂, B(OR)₂ or an acid isostere;

R is alkyl;

when n is not zero, R₃, R₄, R₅ or R₈, or R₆ is each independently selected from:
(1) hydrogen;
(2) substituted or unsubstituted alkyl or ether having 1 to 12 carbon atoms,
(3) a substituted amine, or
(4) —(CH₂)ₘ G, wherein m is 1 to 12 and G is independently selected from:
   (a) cycloalkyl containing 3 to 6 carbon atoms,
   (b) aryl or heteroaryl,
   (c) CF₃, CF₂H or CFH₂, or
   (d) a heterocycle,
provided that R₃, R₄, R₅, R₈, or R₆ are not all hydrogen;

or pharmaceutically acceptable salts or stereoisomers thereof.

In other embodiments the invention is a method for treating a viral disease in a subject comprising administering to said subject a therapeutically effective dose of a compound selected from the group consisting of: 5-[(3-cyanophenyl)methyl]-2-fluoro-7-hexyl-5H,6H,7H,8H,9H,10H-cyclohepta[b]indole-4-carboxylic acid, 5-[(6-cyanopyridin-2-yl)methyl]-7-hexyl-5H,6H,7H,8H,9H,10H-cyclohepta[b]indole-4-carboxylic acid, 5-[(6-carbamoylpyridin-2-yl)methyl]-7-hexyl-5H,6H,7H,8H,9H,10H-cyclohepta[b]indole-4-carboxylic acid, 6-({4-carboxy-7-hexyl-5H,6H,7H,8H,9H,10H-cyclohepta[b]indol-5-yl}methyl)pyridine-2-carboxylic acid, 5-[(3-cyano-2-fluorophenyl)methyl]-7-hexyl-5H,6H,7H,8H,9H,10H-cyclohepta[b]indole-4-carboxylic acid, 5-[(1,3-benzoxazol-6-yl)methyl]-7-hexyl-5H,6H,7H,8H,9H,10H-cyclohepta[b]indole-4-carboxylic acid, 5-[(1,3-benzoxazol-5-yl)methyl]-7-hexyl-5H,6H,7H,8H,9H,10H-cyclohepta[b]indole-4-carboxylic acid, 5-[(6-fluoropyridin-2-yl)methyl]-7-hexyl-5H,6H,7H,8H,9H,10H-cyclohepta[b]indole-4-carboxylic acid, 5-[(2-fluoropyridin-4-yl)methyl]-7-hexyl-5H,6H,7H,8H,9H,10H-cyclohepta[b]indole-4-carboxylic acid, 7-hexyl-5-{[6-(trifluoromethyl)pyridin-2-yl]methyl}-5H,6H,7H,8H,9H,10H-cyclohepta[b]indole-4-carboxylic acid, 7-hexyl-5-{[2-(trifluoromethyl)pyridin-4-yl]methyl}-5H,6H,7H,8H,9H,10H-cyclohepta[b]indole-4-carboxylic acid, 5-[(5-cyanopyridin-3-yl)methyl]-7-hexyl-5H,6H,7H,8H,9H,10H-cyclohepta[b]indole-4-carboxylic acid, 5-[(5-cyanothiophen-2-yl)methyl]-7-hexyl-5H,6H,7H,8H,9H,10H-cyclohepta[b]indole-4-carboxylic acid, 5-[(4-cyanothiophen-2-yl)methyl]-7-hexyl-5H,6H,7H,8H,9H,10H-cyclohepta[b]indole-4-carboxylic acid, 5-[(5-cyanofuran-2-yl)methyl]-7-hexyl-5H,6H,7H,8H,9H,10H-cyclohepta[b]indole-4-carboxylic acid, 5-[(3,5-dimethyl-1,2-oxazol-4-yl)methyl]-7-hexyl-5H,6H,7H,8H,9H,10H-cyclohepta[b]indole-4-carboxylic acid, 5-(3-cyanobenzoyl)-7-hexyl-5H,6H,7H,8H,9H,10H-cyclohepta[b]indole-4-carboxylic acid, 5-[(1,3-benzoxazol-7-yl)methyl]-7-hexyl-5H,6H,7H,8H,9H,10H-cyclohepta[b]indole-4-carboxylic acid, 5-[(5-cyanothiophen-3-yl)methyl]-7-hexyl-5H,6H,7H,8H,9H,10H-cyclohepta[b]indole-4-carboxylic acid, 7-hexyl-5-[(1H-indol-4-yl)methyl]-5H,6H,7H,8H,9H,10H-cyclohepta[b]indole-4-carboxylic acid, 5-[(3-carbamoylphenyl)methyl]-7-propyl-5H,6H,7H,8H,9H,10H-cyclohepta[b]indole-4-carboxylic acid, 7-butyl-5-[(3-carbamoylphenyl)methyl]-5H,6H,7H,8H,9H,10H-cyclohepta[b]indole-4-carboxylic acid, 7-butyl-5-[(3-cyanophenyl)methyl]-5H,6H,7H,8H,9H,10H-cyclohepta[b]indole-4-carboxylic acid, 7-butyl-5-[(pyridin-3-yl)methyl]-5H,6H,7H,8H,9H,10H-cyclohepta[b]indole-4-carboxylic acid, 7-butyl-5-[(3-methylphenyl)methyl]-5H,6H,7H,8H,9H,10H-cyclohepta[b]indole-4-carboxylic acid, 7-butyl-5-[(3-methoxyphenyl)methyl]-5H,6H,7H,8H,9H,10H-cyclohepta[b]indole-4-carboxylic acid, 7-butyl-5-[(3-chlorophenyl)methyl]-5H,6H,7H,8H,9H,10H-cyclohepta[b]indole-4-carboxylic acid, 7-butyl-5-[(3-hydroxyphenyl)methyl]-5H,6H,7H,8H,9H,10H-cyclohepta[b]indole-4-carboxylic acid, 7-butyl-5-[(2-methoxypyridin-4-yl)methyl]-5H,6H,7H,8H,9H,10H-cyclohepta[b]indole-4-carboxylic acid, 7-butyl-5-[(4-carbamoylphenyl)methyl]-5H,6H,7H,8H,9H,10H-cyclohepta[b]indole-4-carboxylic acid, 7-butyl-5-[(2-carbamoylphenyl)methyl]-5H,6H,7H,8H,9H,10H-cyclohepta[b]indole-4-carboxylic acid, 7-butyl-5-[(4-methylphenyl)methyl]-5H,6H,7H,8H,9H,10H-cyclohepta[b]indole-4-carboxylic acid, 7-butyl-5-[(2-cyanophenyl)methyl]-5H,6H,7H,8H,9H,10H-cyclohepta[b]indole-4-carboxylic acid, 7-butyl-5-[(2-methylphenyl)methyl]-5H,6H,7H,8H,9H,10H-cyclohepta[b]indole-4-carboxylic acid, 7-butyl-5-[(2-fluorophenyl)methyl]-5H,6H,7H,8H,9H,10H-cyclohepta[b]indole-4-carboxylic acid, 5-[(3-carbamoylphenyl)methyl]-7-pentyl-5H,6H,7H,8H,9H,10H-cyclohepta[b]indole-4-carboxylic acid, 5-[(3-cyanophenyl)methyl]-7-pentyl-5H,6H,7H,8H,9H,10H-cyclohepta[b]indole-4-carboxylic acid, 5-[(3-carbamoylphenyl)methyl]-7-(2-phenylethyl)-5H,6H,7H,8H,9H,10H-cyclohepta[b]indole-4-carboxylic acid, 5-[(3-cyanophenyl)methyl]-7-(2-phenylethyl)-5H,6H,7H,8H,9H,10H-cyclohepta[b]indole-4-carboxylic acid, 5-[(3-carbamoylphenyl)methyl]-7-hexyl-5H,6H,7H,8H,9H,10H-cyclohepta[b]indole-4-carboxylic acid, 5-[(3-cyanophenyl)methyl]-7-hexyl-5H,6H,7H,8H,9H,10H-cyclohepta[b]indole-4-carboxylic acid, 5-[(3-carbamoylphenyl)methyl]-7-octyl-5H,6H,7H,8H,9H,10H-cyclohepta[b]indole-4-carboxylic acid, 5-[(3-cyanophenyl)methyl]-7-octyl-5H,6H,7H,8H,9H,10H-cyclohepta[b]indole-4-carboxylic acid, 5-[(3-fluorophenyl)methyl]-7-hexyl-5H,6H,7H,8H,9H,10H-cyclohepta[b]indole-4-carboxylic acid, 7-hexyl-5-[(pyridin-3-yl)methyl]-5H,6H,7H,8H,9H,10H-cyclohepta[b]indole-4-carboxylic acid, 7-hexyl-5-[(3-methylphenyl)methyl]-5H,6H,7H,8H,9H,10H-cyclohepta[b]indole-4-carboxylic acid, 7-hexyl-5-[(3-methoxyphenyl)methyl]-5H,6H,7H,8H,9H,10H-cyclohepta[b]indole-4-carboxylic acid, 5-[(3-chlorophenyl)methyl]-7-hexyl-5H,6H,7H,8H,9H,10H-cyclohepta[b]indole-4-carboxylic acid, 7-hexyl-5-[(2-methoxypyridin-4-yl)methyl]-5H,6H,7H,8H,9H,10H-cyclohepta[b]indole-4-carboxylic acid, 5-[(3-carboxyphenyl)methyl]-7-hexyl-5H,6H,7H,8H,9H,10H-cyclohepta[b]indole-4-carboxylic acid, 5-[(4-carbamoylphenyl)methyl]-7-hexyl-5H,6H,7H,8H,9H,10H-cyclohepta[b]indole-4-carboxylic acid, 5-[(2-carbamoylphenyl)methyl]-7-hexyl-5H,6H,7H,8H,9H,10H-cyclohepta[b]indole-4-carboxylic acid, 7-hexyl-5-[(4-methylphenyl)methyl]-5H,6H,7H,8H,9H,10H-cyclohepta[b]indole-4-carboxylic acid, 5-[(4-cyanophenyl)methyl]-7-hexyl-5H,6H,7H,8H,9H,10H-cyclohepta[b]indole-4-carboxylic acid, 5-[(2-cyanophenyl)methyl]-7-hexyl-5H,6H,7H,8H,9H,10H-cyclohepta[b]indole-4-carboxylic acid, 7-hexyl-5-[(2-methylphenyl)methyl]-5H,6H,7H,8H,9H,10H-cyclohepta[b]indole-4-carboxylic acid, 5-[(2-fluorophenyl)methyl]-7-hexyl-5H,6H,7H,8H,9H,10H-cyclohepta[b]indole-4-carboxylic acid, 5-[(4-fluorophenyl)methyl]-7-hexyl-5H,6H,7H,8H,9H,10H-cyclohepta[b]indole-4-carboxylic acid, 9-[(3-carbamoylphenyl)methyl]-2-hexyl-2,3,4,9-tetrahydro-1H-carbazole-8-carboxylic acid, 9-[(3-cyanophenyl)methyl]-2-hexyl-2,3,4,9-tetrahydro-1H-carbazole-8-carboxylic acid, 9-[(3-carboxyphenyl)methyl]-2-hexyl-2,3,4,9-tetrahydro-1H-carbazole-8-carboxylic acid, 9-[(3-fluorophenyl)methyl]-2-hexyl-2,3,4,9-tetrahydro-1H-carbazole-8-carboxylic acid, 2-hexyl-9-[(pyridin-3-yl)methyl]-2,3,4,9-tetrahydro-1H-carbazole-8-carboxylic acid, 2-hexyl-9-[(3-methylphenyl)methyl]-2,3,4,9-tetrahydro-1H-carbazole-8-carboxylic acid, 2-hexyl-9-[(3-methoxyphenyl)methyl]-2,3,4,9-tetrahydro-1H-carbazole-8-carboxylic acid, 9-[(3-chlorophenyl)methyl]-2-hexyl-2,3,4,9-tetrahydro-1H-carbazole-8-carboxylic acid, 2-hexyl-9-[(3-hydroxyphenyl)methyl]-2,3,4,9-tetrahydro-1H-carbazole-8-carboxylic acid, 2-hexyl-9-[(2-methoxypyridin-4-yl)methyl]-2,3,4,9-tetrahydro-1H-carbazole-8-carboxylic acid, 9-[(3-carboxyphenyl)methyl]-2-hexyl-2,3,4,9-tetrahydro-1H-carbazole-8-carboxylic acid, 9-[(4-carbamoylphenyl)methyl]-2-hexyl-2,3,4,9-tetrahydro-1H-carbazole-8-carboxylic acid, 9-[(2-carbamoylphenyl)methyl]-2-hexyl-2,3,4,9-tetrahydro-1H-carbazole-8-carboxylic acid, 2-hexyl-9-[(4-methylphenyl)methyl]-2,3,4,9-tetrahydro-1H-carbazole-8-carboxylic acid, 9-[(4-cyanophenyl)methyl]-2-hexyl-2,3,4,9-tetrahydro-1H-carbazole-8-carboxylic acid, 9-[(2-cyanophenyl)methyl]-2-hexyl-2,3,4,9-tetrahydro-1H-carbazole-8-carboxylic acid, 2-hexyl-9-[(2-methylphenyl)methyl]-2,3,4,9-tetrahydro-1H-carbazole-8-carboxylic acid, 9-[(2-fluorophenyl)methyl]-2-hexyl-2,3,4,9-tetrahydro-1H-carbazole-8-carboxylic acid, 9-[(3-carbamoylphenyl)methyl]-2-(2-phenylethyl)-2,3,4,9-tetrahydro-1H-carbazole-8-carboxylic acid, 9-[(3-cyanophenyl)methyl]-2-(2-phenylethyl)-2,3,4,9-tetrahydro-1H-carbazole-8-carboxylic acid, 9-[(3-carbamoylphenyl)methyl]-4-(2-phenylethyl)-2,3,4,9-tetrahydro-1H-carbazole-8-carboxylic acid, 9-[(3-cyanophenyl)methyl]-4-(2-phenylethyl)-2,3,4,9-tetrahydro-1H-carbazole-8-carboxylic acid, 9-[(3-carbamoylphenyl)methyl]-2-propyl-2,3,4,9-tetrahydro-1H-carbazole-8-carboxylic acid, 9-[(3-cyanophenyl)methyl]-2-propyl-2,3,4,9-tetrahydro-1H-carbazole-8-carboxylic acid, 2-butyl-9-[(3-carbamoylphenyl)methyl]-2,3,4,9-tetrahydro-1H-carbazole-8-carboxylic acid, 9-[(3-carbamoylphenyl)methyl]-2-pentyl-2,3,4,9-tetrahydro-1H-carbazole-8-carboxylic acid, 9-[(3-cyanophenyl)methyl]-2-pentyl-2,3,4,9-tetrahydro-1H-carbazole-8-carboxylic acid, 1-butyl-9-[(3-carbamoylphenyl)methyl]-2,3,4,9-tetrahydro-1H-carbazole-8-carboxylic acid, 9-[(3-carbamoylphenyl)methyl]-1-(pentyloxy)-2,3,4,9-tetrahydro-1H-carbazole-8-carboxylic acid, 9-[(3-cyanophenyl)methyl]-1-(pentyloxy)-2,3,4,9-tetrahydro-1H-carbazole-8-carboxylic acid, 1-butyl-9-[(3-cyanophenyl)methyl]-2,3,4,9-tetrahydro-1H-carbazole-8-carboxylic acid, 6-butyl-5-[(3-carbamoylphenyl)methyl]-5H,6H,7H,8H,9H,10H-cyclohepta[b]indole-4-carboxylic acid, 6-butyl-5-[(3-cyanophenyl)methyl]-5H,6H,7H,8H,9H,10H-cyclohepta[b]indole-4-carboxylic acid, 9-[(3-carbamoylphenyl)methyl]-1-ethyl-2,3,4,9-tetrahydro-1H-carbazole-8-carboxylic acid, 9-[(3-cyanophenyl)methyl]-1-ethyl-2,3,4,9-tetrahydro-1H-carbazole-8-carboxylic acid, 9-[(3-carboxyphenyl)methyl]-1-ethyl-2,3,4,9-tetrahydro-1H-carbazole-8-carboxylic acid, 9-[(3-carbamoylphenyl)methyl]-1-propoxy-2,3,4,9-tetrahydro-1H-carbazole-8-carboxylic acid, 9-[(3-carbamoylphenyl)methyl]-4-ethyl-2,3,4,9-tetrahydro-1H-carbazole-8-carboxylic acid, 4-[(3-carbamoylphenyl)methyl]-3-ethyl-1H,2H,3H,4H-cyclopenta[b]indole-5-carboxylic acid, 4-[(3-cyanophenyl)methyl]-3-ethyl-1H,2H,3H,4H-cyclopenta[b]indole-5-carboxylic acid, 3-butyl-4-[(3-carbamoylphenyl)methyl]-1H,2H,3H,4H-cyclopenta[b]indole-5-carboxylic acid, 3-butyl-4-[(3-cyanophenyl)methyl]-1H,2H,3H,4H-cyclopenta[b]indole-5-carboxylic acid, 2-butyl-4-[(3-carbamoylphenyl)methyl]-1H,2H,3H,4H-cyclopenta[b]indole-5-carboxylic acid, 2-butyl-4-[(3-cyanophenyl)methyl]-1H,2H,3H,4H-cyclopenta[b]indole-5-carboxylic acid, 5-[(3-carbamoylphenyl)methyl]-10-ethyl-5H,6H,7H,8H,9H,10H-cyclohepta[b]indole-4-carboxylic acid, 5-[(3-cyanophenyl)methyl]-10-ethyl-5H,6H,7H,8H,9H,10H-cyclohepta[b]indole-4-carboxylic acid, 5-[(3-carbamoylphenyl)methyl]-10-propyl-5H,6H,7H,8H,9H,10H-cyclohepta[b]indole-4-carboxylic acid, 5-[(3-cyanophenyl)methyl]-10-propyl-5H,6H,7H,8H,9H,10H-cyclohepta[b]indole-4-carboxylic acid, 9-[(3-carboxyphenyl)methyl]-4-ethyl-2,3,4,9-tetrahydro-1H-carbazole-8-carboxylic acid, 9-[(3-carbamoylphenyl)methyl]-3-ethyl-2,3,4,9-tetrahydro-1H-carbazole-8-carboxylic acid, 10-butyl-5-[(3-carbamoylphenyl)methyl]-5H,6H,7H,8H,9H,10H-cyclohepta[b]indole-4-carboxylic acid, 10-butyl-5-[(3-cyanophenyl)methyl]-5H,6H,7H,8H,9H,10H-cyclohepta[b]indole-4-carboxylic acid, 5-[(3-carbamoylphenyl)methyl]-10-pentyl-5H,6H,7H,8H,9H,10H-cyclohepta[b]indole-4-carboxylic acid, 5-[(3-cyanophenyl)methyl]-10-pentyl-5H,6H,7H,8H,9H,10H-cyclohepta[b]indole-4-carboxylic acid, 4-[(3-carbamoylphenyl)methyl]-2-pentyl-1H,2H,3H,4H-cyclopenta[b]indole-5-carboxylic acid, 4-[(3-cyanophenyl)methyl]-2-pentyl-1H,2H,3H,4H-cyclopenta[b]indole-5-carboxylic acid, 9-[(3-carbamoylphenyl)methyl]-2-ethyl-2,3,4,9-tetrahydro-1H-carbazole-8-carboxylic acid, 9-[(3-carboxyphenyl)methyl]-2-ethyl-2,3,4,9-tetrahydro-1H-carbazole-8-carboxylic acid, 5-[(3-carbamoylphenyl)methyl]-7-ethyl-5H,6H,7H,8H,9H,10H-cyclohepta[b]indole-4-carboxylic acid, 5-[(3-cyanophenyl)methyl]-7-ethyl-5H,6H,7H,8H,9H,10H-cyclohepta[b]indole-4-carboxylic acid, 9-[(3-cyanophenyl)methyl]-3-ethyl-2,3,4,9-tetrahydro-1H-carbazole-8-carboxylic acid, 9-[(3-carbamoylphenyl)methyl]-4-propyl-2,3,4,9-tetrahydro-1H-carbazole-8-carboxylic acid, 9-[(3-carbamoylphenyl)methyl]-3-propyl-2,3,4,9-tetrahydro-1H-carbazole-8-carboxylic acid, 9-[(3-cyanophenyl)methyl]-3-propyl-2,3,4,9-tetrahydro-1H-carbazole-8-carboxylic acid, 4-butyl-9-[(3-carbamoylphenyl)methyl]-2,3,4,9-tetrahydro-1H-carbazole-8-carboxylic acid, 4-butyl-9-[(3-cyanophenyl)methyl]-2,3,4,9-tetrahydro-1H-carbazole-8-carboxylic acid, 3-butyl-9-[(3-carbamoylphenyl)methyl]-2,3,4,9-tetrahydro-1H-carbazole-8-carboxylic acid, 3-butyl-9-[(3-cyanophenyl)methyl]-2,3,4,9-tetrahydro-1H-carbazole-8-carboxylic acid, 9-[(3-carbamoylphenyl)methyl]-4-pentyl-2,3,4,9-tetrahydro-1H-carbazole-8-carboxylic acid, 9-[(3-cyanophenyl)methyl]-4-pentyl-2,3,4,9-tetrahydro-1H-carbazole-8-carboxylic acid, 9-[(3-carbamoylphenyl)methyl]-3-pentyl-2,3,4,9-tetrahydro-1H-carbazole-8-carboxylic acid, 9-[(3-cyanophenyl)methyl]-3-pentyl-2,3,4,9-tetrahydro-1H-carbazole-8-carboxylic acid, 4-[(3-carbamoylphenyl)methyl]-3-propyl-1H,2H,3H,4H-cyclopenta[b]indole-5-carboxylic acid, 4-[(3-cyanophenyl)methyl]-3-propyl-1H,2H,3H,4H-cyclopenta[b]indole-5-carboxylic acid, 2-({7-butyl-5-[(3-carbamoylphenyl)methyl]-5H,6H,7H,8H,9H,10H-cyclohepta[b]indol-4-yl}formamido)acetic acid, 2-({7-butyl-5-[(3-cyanophenyl)methyl]-5H,6H,7H,8H,9H,10H-cyclohepta[b]indol-4-yl}formamido)acetic acid, 7-butyl-5-[(3-carbamoylphenyl)methyl]-N-(2-hydroxyethyl)-5H,6H,7H,8H,9H,10H-cyclohepta[b]indole-4-carboxamide, 7-butyl-5-[(3-cyanophenyl)methyl]-N-(2-hydroxyethyl)-5H,6H,7H,8H,9H,10H-cyclohepta[b]indole-4-carboxamide, 7-butyl-5-[(3-fluorophenyl)methyl]-5H,6H,7H,8H,9H,10H-cyclohepta[b]indole-4-carboxylic acid, and 7-butyl-5-[(3-carboxyphenyl)methyl]-5H,6H,7H,8H,9H,10H-cyclohepta[b]indole-4-carboxylic acid, or pharmaceutically acceptable salts or stereoisomers thereof.

Yet in other embodiments the invention is a method for treating a viral disease in a subject comprising administering to said subject a therapeutically effective dose of a compound selected from the group consisting of: 2-((2′-(5-Ethyl-3,4-diphenyl-1H-pyrazol-1-yl)-[1,1′-biphenyl]-3-yl)oxy)acetic acid, 6-chloro-4-phenyl-2-(piperidin-1-yl)-3-(1H-tetrazol-5-yl)quinoline, 5-((3-chloro-2-methylphenoxy)methyl)-2-phenyl-[1,2,4]triazolo[1,5-a]pyrimidin-7(4H)-one, and 3-(5-cyclopropyl-3-(3,5-dimethyl-1H-pyrazol-4-yl)-2-(3-isopropoxyphenyl)-1H-indol-1-yl)propanoic acid, or pharmaceutically acceptable salts or stereoisomers thereof. It should be appreciated that WO 00/59506, U.S. 2013/0116234 A1, WO 2010/056631, PCT/US21/39470, U.S. 2013/0116231 A1, Sulsky et al., Bioorg Med Chem Lett. 2007 Jun. 15; 17(12):3511-5. doi: 10.1016/j.bmc.2006.12.044. Epub 2006 Dec. 21, Kuhne et al., Bioorg Med Chem Lett. 2016 Oct. 15; 26(20):5092-5097. doi: 10.1016/j.bmcl.2016.08.071. Epub 2016 Aug. 22.), and Lan et al., J Lipid Res. 2011 April; 52(4):646-56. doi: 10.1194/jlr.M012757. Epub 2011 Feb. 4.), Tagami et al., ACS Med Chem Lett. 2016 Apr. 14; 7(4): 435-439. doi: 10.1021/acsmedchemlett.6b00040) are hereby incorporated by reference in their entirety.

It is an object of the present invention to utilize the compounds described herein in the treatment of viral disorders by acting on the fatty acid binding protein (FABP4).

Yet another object of the present invention is a pharmaceutical composition comprising a compounds herein as active ingredient, in combination with a pharmaceutically acceptable diluent or carrier for use in the treatment of viral disorders by acting on FABP4. Here, the pharmaceutical composition can further comprise an additional therapeutically active agent.

Yet another object of the present invention is a method for the treatment of viral disorders by acting on the FABP4, which comprises administering to a subject in need of such treatment (preferably a human) an effective amount of the compounds herein, including, optionally, the co-administration with other therapeutic agents, either as a single (or multiple) dosing, and either simultaneously or sequentially.

Yet another object of the present invention is the use of the compounds herein for the manufacture of a medicament for use in the treatment of viral disorders by acting on the fatty acid binding protein FABP4.

Examples of such disorders include common cold, SARS, and COVID-19.

Other features and advantages of the invention will be apparent from the detailed description and the claims.

### BRIEF DESCRIPTION OF THE DRAWINGS

FIG. 1A and FIG. 1B depict graphical charts showing a virus yield obtained from cells treated with corresponding compounds and vehicle control (DMSO), according to at least some embodiments disclosed herein.

### DESCRIPTION OF THE PREFERRED EMBODIMENTS

Reference will now be made in detail to each embodiment of the present invention. Such embodiments are provided by way of explanation of the present invention, which is not intended to be limited thereto. In fact, those of ordinary skill in the art may appreciate upon reading the present specification and viewing the present drawings that various modifications and variations can be.

The invention is a method for treating a viral disease in a subject comprising administering to said subject a therapeutically effective dose of one or more of the compounds described in Formula (I):

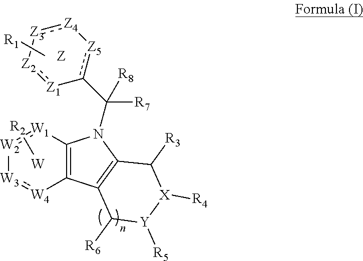

Wherein:

W₁₋₄ and Z₁-Z₅ are each independently —C, —CH, CH₂, O, S, or N;

X is independently CH₂, N or CHR₄;

Y is independently CH₂, or CHR₅;

n is a number between 0 and 3;

One or more R₁'s on the ring Z are independently selected from the group consisting of: CN, OH, COOH, OCH₃, CF₃, CONH₂, B(OH)₂, B(OR)₂, an acid isostere, a substituted amine, ethers, and a halogen, substituted or unsubstituted alkyl, substituted or unsubstituted cyclic or heterocyclic, substituted or unsubstituted cycloaryl or cychloheteroaryl, wherein the substituted cycloaryl or cychloheteroaryl may be substituted with hydrogen, CN, OH, COOH, OCH₃, CF₃, CONH₂, B(OH)₂, B(OR)₂, an acid isostere, a substituted amine, ethers, and a halogen, substituted or unsubstituted alkyl, substituted or unsubstituted cyclic or heterocyclic, substituted or unsubstituted cycloaryl or cychloheteroaryl, and SO₂NH₂;

One or more R₂'s on the ring W are independently selected from the group consisting of: CN, OH, CHF2, CH2F, CF₃, COOH, CONH₂, B(OH)₂, B(OR)₂, an acid isostere, a halogen, and a bicyclic heteroaryl;

R₇ is hydrogen or CN, COOH, CONH₂, B(OH)₂, B(OR)₂ or an acid isostere;

R is alkyl;

when n is not zero, R₃, R₄, R₅ or R₈, or R₆ is each independently selected from:
(1) hydrogen;
(2) substituted or unsubstituted alkyl or ether having 1 to 12 carbon atoms,
(3) a substituted amine, or
(4) —(CH₂)ₘ G, wherein m is 1 to 12 and G is independently selected from:
   (a) cycloalkyl containing 3 to 6 carbon atoms,
   (b) aryl or heteroaryl,
   (c) CF₃, CF₂H or CFH₂, or
   (d) a heterocycle,
provided that R₃, R₄, R₅, R₈, or R₆ are not all hydrogen;

or pharmaceutically acceptable salts or stereoisomers thereof.

In examples, where R₁ and R₂ are both present, each is independently CN, COOH, or CONH₂.

In examples, Formula I includes more than one R₁ and more than one R₂.

In examples, R₃, R₄, R₅ R₈, or R₆, when n is not zero, are each independently alkyl having 4 carbon atoms.

In examples, R₃, R₄, R₅ R₈, or R₆, when n is not zero, are each independently alkyl having 5 carbon atoms.

In examples, R₃, R₄, R₅ R₈, or R₆, when n is not zero, are each independently alkyl having 6 carbon atoms.

In examples, the ring Z contains Z₁-Z₄.

In examples, the ring Z contains Z₁-Z₅.

In examples, the R₁ on the ring Z is the halogen.

In examples, the R₁ on the ring Z is the CN.

In examples, the R₁ on the ring Z is the CF₃.

In examples, R₂ on the ring W is the halogen.

In examples, the R₁ on the ring Z comprise the CN and/or the halogen.

In examples, R₁ on the ring Z comprise the CN and/or the halogen, and the R₂ on the ring W comprise another halogen. In examples, the halogen is identical to the other halogen. In other examples, the halogen differs from the other halogen.

In examples, each of the one or more compounds are a pure optical isomer.

The invention also describes a method for treating a viral disease in a subject comprising administering to said subject a therapeutically effective dose of one or more compounds described in Formula (II):

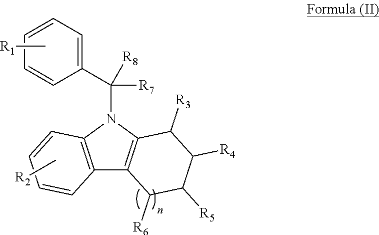

Wherein:

n=0, 1, or 2;

R₁ is selected from the group consisting of: CN, COOH, CONH₂, B(OH)₂, B(OR)₂, an acid isostere, and a halogen;

R₂ is selected from the group consisting of: CN, COOH, CONH₂, B(OH)₂, B(OR)₂, an acid isostere, a halogen, and a bicyclic compound;

R₇ is hydrogen or CN, COOH, CONH₂, B(OH)₂, B(OR)₂ or an acid isostere;

R is alkyl;

R₃, R₄, R₅ or R₈, or R₆ when n is not zero, is each independently selected from:
(1) hydrogen;
(2) alkyl having 1 to 12 carbon atoms, or
(3) —(CH₂)ₘG, wherein m is 1 to 12 and G is independently selected from:
   (a) cycloalkyl containing 3 to 6 carbon atoms,
   (b) aryl or heteroaryl, or
   (c) CF₃, CF₂H or CFH₂;
provided that G is not a nitrogen or oxygen-containing group; and
provided that R₃, R₄, R₅, R₈, or R₆ are not all hydrogen;

or pharmaceutically acceptable salts thereof.

In examples, when R₁ and R₂ are both present, each is independently CN, COOH, or CONH₂.

In examples, Formula II includes more than one R₁ and more than one R₂.

In examples, R₃, R₄, R₅, R₈, or R₆, when n is not zero, are each independently alkyl having 4 carbon atoms.

In examples, R₃, R₄, R₅, R₈, or R₆, when n is not zero, are each independently alkyl having 5 carbon atoms.

In examples, R₃, R₄, R₅, R₈, or R₆, when n is not zero, are each independently alkyl having 6 carbon atoms.

The present invention also describes a method for treating a viral disease in a subject comprising administering to said subject a therapeutically effective dose of one or more of the compounds described in Formula (III):

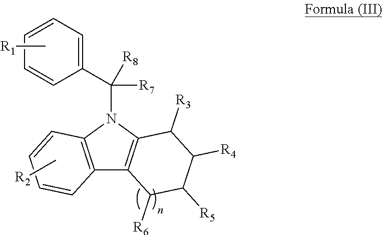

### Wherein:

n=0, 1, or 2;
R₁ and R₂ are each independently halogen, alkyl, cycloalkyl, aryl, heteroaryl, CN, COOH, CONH₂, B(OH)₂, B(OR)₂, or an acid isostere;

R₇ is hydrogen, alkyl, cycloalkyl, aryl, heteroaryl, CN, COOH, CONH₂, B(OH)₂, B(OR)₂ or an acid isostere;

R is alkyl;

R₃, R₄, R₅ or R₈, or R₆ when n is not zero, are each independently selected from:
(1) hydrogen;
(2) alkyl having 1 to 12 carbon atoms, or
(3) —(CH₂)ₘG, wherein m is 1 to 12 and G is independently selected from:
   (a) cycloalkyl containing 3 to 6 carbon atoms;
   (b) aryl or heteroaryl; or
   (c) CF₃, CF₂H, or CFH₂;

provided that R₃, R₄, R₅, R₈, or R₆ are not all hydrogen;

or pharmaceutically acceptable salts or stereoisomers thereof.

In examples, R₃, R₄, R₅, R₈, or R₆, when n is 1, are each independently alkyl having 4 carbon atoms.

In examples, R₃, R₄, R₅, R₈, or R₆, when n is 1, are each independently alkyl having 5 carbon atoms.

In examples, R₃, R₄, R₅, R₈, or R₆, when n is 1, are each independently alkyl having 6 carbon atoms.

The present invention also describes a method for treating a viral disease in a subject comprising administering to said subject a therapeutically effective dose of one or more of the of compounds described in Formula (IV):

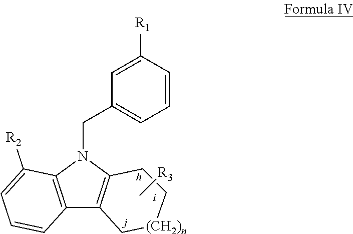

Wherein:

n=0, 1 or 2;

R₁ and R₂ are each independently CN, COOH or CONH₂;

R₃ is independently selected from:
(1) alkyl having 1 to 12 carbon atoms;
(2) —(CH₂)ₘG, wherein m is 1 to 12 and G is independently selected from:
   (a) cycloalkyl containing 3 to 6 carbon atoms;
   (b) phenyl; and
provided that G is not a nitrogen or oxygen-containing group;

or pharmaceutically acceptable salts thereof.

In examples, when n=0, R₃ is attached to the h-, i- or j-position.

In examples, when n=1, R₃ is attached to the h-, i- or j-position.

In examples, when n=2, R₃ is attached to the h-, i- or j-position.

In other embodiments the invention is a method for treating a viral disease in a subject comprising administering to said subject a therapeutically effective dose of a compound from PCT/US21/39470 filed on Jun. 28, 2021, U.S. Published Patent Application No. 2013/0116231 A1, or WO 2010/056631 A1, the entire contents of which are hereby incorporated by reference in their entirety.

In other examples, the invention provides a method for treating a viral disease in a subject comprising administering to said subject a therapeutically effective dose of one or more of the compounds selected from the group consisting of: 5-[(3-cyanophenyl)methyl]-2-fluoro-7-hexyl-5H,6H,7H,8H,9H,10H-cyclohepta[b]indole-4-carboxylic acid, 5-[(6-cyanopyridin-2-yl)methyl]-7-hexyl-5H,6H,7H,8H,9H,10H-cyclohepta[b]indole-4-carboxylic acid, 5-[(6-carbamoylpyridin-2-yl)methyl]-7-hexyl-5H,6H,7H,8H,9H,10H-cyclohepta[b]indole-4-carboxylic acid, 6-({4-carboxy-7-hexyl-5H,6H,7H,8H,9H,10H-cyclohepta[b]indol-5-yl}methyl)pyridine-2-carboxylic acid, 5-[(3-cyano-2-fluorophenyl)methyl]-7-hexyl-5H,6H,7H,8H,9H,10H-cyclohepta[b]indole-4-carboxylic acid, 5-[(1,3-benzoxazol-6-yl)methyl]-7-hexyl-5H,6H,7H,8H,9H,10H-cyclohepta[b]indole-4-carboxylic acid, 5-[(1,3-benzoxazol-5-yl)methyl]-7-hexyl-5H,6H,7H,8H,9H,10H-cyclohepta[b]indole-4-carboxylic acid, 5-[(6-fluoropyridin-2-yl)methyl]-7-hexyl-5H,6H,7H,8H,9H,10H-cyclohepta[b]indole-4-carboxylic acid, 5-[(2-fluoropyridin-4-yl)methyl]-7-hexyl-5H,6H,7H,8H,9H,10H-cyclohepta[b]indole-4-carboxylic acid, 7-hexyl-5-{[6-(trifluoromethyl)pyridin-2-yl]methyl}-5H,6H,7H,8H,9H,10H-cyclohepta[b]indole-4-carboxylic acid, 7-hexyl-5-{[2-(trifluoromethyl)pyridin-4-yl]methyl}-5H,6H,7H,8H,9H,10H-cyclohepta[b]indole-4-carboxylic acid, 5-[(5-cyanopyridin-3-yl)methyl]-7-hexyl-5H,6H,7H,8H,9H,10H-cyclohepta[b]indole-4-carboxylic acid, 5-[(5-cyanothiophen-2-yl)methyl]-7-hexyl-5H,6H,7H,8H,9H,10H-cyclohepta[b]indole-4-carboxylic acid, 5-[(4-cyanothiophen-2-yl)methyl]-7-hexyl-5H,6H,7H,8H,9H,10H-cyclohepta[b]indole-4-carboxylic acid, 5-[(5-cyanofuran-2-yl)methyl]-7-hexyl-5H,6H,7H,8H,9H,10H-cyclohepta[b]indole-4-carboxylic acid, 5-[(3,5-dimethyl-1,2-oxazol-4-yl)methyl]-7-hexyl-5H,6H,7H,8H,9H,10H-cyclohepta[b]indole-4-carboxylic acid, 5-(3-cyanobenzoyl)-7-hexyl-5H,6H,7H,8H,9H,10H-cyclohepta[b]indole-4-carboxylic acid, 5-[(1,3-benzoxazol-7-yl)methyl]-7-hexyl-5H,6H,7H,8H,9H,10H-cyclohepta[b]indole-4-carboxylic acid, 5-[(5-cyanothiophen-3-yl)methyl]-7-hexyl-5H,6H,7H,8H,9H,10H-cyclohepta[b]indole-4-carboxylic acid, 7-hexyl-5-[(1H-indol-4-yl)methyl]-5H,6H,7H,8H,9H,10H-cyclohepta[b]indole-4-carboxylic acid, 5-[(3-carbamoylphenyl)methyl]-7-propyl-5H,6H,7H,8H,9H,10H-cyclohepta[b]indole-4-carboxylic acid, 7-butyl-5-[(3-carbamoylphenyl)methyl]-5H,6H,7H,8H,9H,10H-cyclohepta[b]indole-4-carboxylic acid, 7-butyl-5-[(3-cyanophenyl)methyl]-5H,6H,7H,8H,9H,10H-cyclohepta[b]indole-4-carboxylic acid, 7-butyl-5-[(pyridin-3-yl)methyl]-5H,6H,7H,8H,9H,10H-cyclohepta[b]indole-4-carboxylic acid, 7-butyl-5-[(3-methylphenyl)methyl]-5H,6H,7H,8H,9H,10H-cyclohepta[b]indole-4-carboxylic acid, 7-butyl-5-[(3-methoxyphenyl)methyl]-5H,6H,7H,8H,9H,10H-cyclohepta[b]indole-4-carboxylic acid, 7-butyl-5-[(3-chlorophenyl)methyl]-5H,6H,7H,8H,9H,10H-cyclohepta[b]indole-4-carboxylic acid, 7-butyl-5-[(3-hydroxyphenyl)methyl]-5H,6H,7H,8H,9H,10H-cyclohepta[b]indole-4-carboxylic acid, 7-butyl-5-[(2-methoxypyridin-4-yl)methyl]-5H,6H,7H,8H,9H,10H-cyclohepta[b]indole-4-carboxylic acid, 7-butyl-5-[(4-carbamoylphenyl)methyl]-5H,6H,7H,8H,9H,10H-cyclohepta[b]indole-4-carboxylic acid, 7-butyl-5-[(2-carbamoylphenyl)methyl]-5H,6H,7H,8H,9H,10H-cyclohepta[b]indole-4-carboxylic acid, 7-butyl-5-[(4-methylphenyl)methyl]-5H,6H,7H,8H,9H,10H-cyclohepta[b]indole-4-carboxylic acid, 7-butyl-5-[(2-cyanophenyl)methyl]-5H,6H,7H,8H,9H,10H-cyclohepta[b]indole-4-carboxylic acid, 7-butyl-5-[(2-methylphenyl)methyl]-5H,6H,7H,8H,9H,10H-cyclohepta[b]indole-4-carboxylic acid, 7-butyl-5-[(2-fluorophenyl)methyl]-5H,6H,7H,8H,9H,10H-cyclohepta[b]indole-4-carboxylic acid, 5-[(3-carbamoylphenyl)methyl]-7-pentyl-5H,6H,7H,8H,9H,10H-cyclohepta[b]indole-4-carboxylic acid, 5-[(3-cyanophenyl)methyl]-7-pentyl-5H,6H,7H,8H,9H,10H-cyclohepta[b]indole-4-carboxylic acid, 5-[(3-carbamoylphenyl)methyl]-7-(2-phenylethyl)-5H,6H,7H,8H,9H,10H-cyclohepta[b]indole-4-carboxylic acid, 5-[(3-cyanophenyl)methyl]-7-(2-phenylethyl)-5H,6H,7H,8H,9H,10H-cyclohepta[b]indole-4-carboxylic acid, 5-[(3-carbamoylphenyl)methyl]-7-hexyl-5H,6H,7H,8H,9H,10H-cyclohepta[b]indole-4-carboxylic acid, 5-[(3-cyanophenyl)methyl]-7-hexyl-5H,6H,7H,8H,9H,10H-cyclohepta[b]indole-4-carboxylic acid, 5-[(3-carbamoylphenyl)methyl]-7-octyl-5H,6H,7H,8H,9H,10H-cyclohepta[b]indole-4-carboxylic acid, 5-[(3-cyanophenyl)methyl]-7-octyl-5H,6H,7H,8H,9H,10H-cyclohepta[b]indole-4-carboxylic acid, 5-[(3-fluorophenyl)methyl]-7-hexyl-5H,6H,7H,8H,9H,10H-cyclohepta[b]indole-4-carboxylic acid, 7-hexyl-5-[(pyridin-3-yl)methyl]-5H,6H,7H,8H,9H,10H-cyclohepta[b]indole-4-carboxylic acid, 7-hexyl-5-[(3-methylphenyl)methyl]-5H,6H,7H,8H,9H,10H-cyclohepta[b]indole-4-carboxylic acid, 7-hexyl-5-[(3-methoxyphenyl)methyl]-5H,6H,7H,8H,9H,10H-cyclohepta[b]indole-4-carboxylic acid, 5-[(3-chlorophenyl)methyl]-7-hexyl-5H,6H,7H,8H,9H,10H-cyclohepta[b]indole-4-carboxylic acid, 7-hexyl-5-[(2-methoxypyridin-4-yl)methyl]-5H,6H,7H,8H,9H,10H-cyclohepta[b]indole-4-carboxylic acid, 5-[(3-carboxyphenyl)methyl]-7-hexyl-5H,6H,7H,8H,9H,10H-cyclohepta[b]indole-4-carboxylic acid, 5-[(4-carbamoylphenyl)methyl]-7-hexyl-5H,6H,7H,8H,9H,10H-cyclohepta[b]indole-4-carboxylic acid, 5-[(2-carbamoylphenyl)methyl]-7-hexyl-5H,6H,7H,8H,9H,10H-cyclohepta[b]indole-4-carboxylic acid, 7-hexyl-5-[(4-methylphenyl)methyl]-5H,6H,7H,8H,9H,10H-cyclohepta[b]indole-4-carboxylic acid, 5-[(4-cyanophenyl)methyl]-7-hexyl-5H,6H,7H,8H,9H,10H-cyclohepta[b]indole-4-carboxylic acid, 5-[(2-cyanophenyl)methyl]-7-hexyl-5H,6H,7H,8H,9H,10H-cyclohepta[b]indole-4-carboxylic acid, 7-hexyl-5-[(2-methylphenyl)methyl]-5H,6H,7H,8H,9H,10H-cyclohepta[b]indole-4-carboxylic acid, 5-[(2-fluorophenyl)methyl]-7-hexyl-5H,6H,7H,8H,9H,10H-cyclohepta[b]indole-4-carboxylic acid, 5-[(4-fluorophenyl)methyl]-7-hexyl-5H,6H,7H,8H,9H,10H-cyclohepta[b]indole-4-carboxylic acid, 9-[(3-carbamoylphenyl)methyl]-2-hexyl-2,3,4,9-tetrahydro-1H-carbazole-8-carboxylic acid, 9-[(3-cyanophenyl)methyl]-2-hexyl-2,3,4,9-tetrahydro-1H-carbazole-8-carboxylic acid, 9-[(3-carboxyphenyl)methyl]-2-hexyl-2,3,4,9-tetrahydro-1H-carbazole-8-carboxylic acid, 9-[(3-fluorophenyl)methyl]-2-hexyl-2,3,4,9-tetrahydro-1H-carbazole-8-carboxylic acid, 2-hexyl-9-[(pyridin-3-yl)methyl]-2,3,4,9-tetrahydro-1H-carbazole-8-carboxylic acid, 2-hexyl-9-[(3-methylphenyl)methyl]-2,3,4,9-tetrahydro-1H-carbazole-8-carboxylic acid, 2-hexyl-9-[(3-methoxyphenyl)methyl]-2,3,4,9-tetrahydro-1H-carbazole-8-carboxylic acid, 9-[(3-chlorophenyl)methyl]-2-hexyl-2,3,4,9-tetrahydro-1H-carbazole-8-carboxylic acid, 2-hexyl-9-[(3-hydroxyphenyl)methyl]-2,3,4,9-tetrahydro-1H-carbazole-8-carboxylic acid, 2-hexyl-9-[(2-methoxypyridin-4-yl)methyl]-2,3,4,9-tetrahydro-1H-carbazole-8-carboxylic acid, 9-[(3-carboxyphenyl)methyl]-2-hexyl-2,3,4,9-tetrahydro-1H-carbazole-8-carboxylic acid, 9-[(4-carbamoylphenyl)methyl]-2-hexyl-2,3,4,9-tetrahydro-1H-carbazole-8-carboxylic acid, 9-[(2-carbamoylphenyl)methyl]-2-hexyl-2,3,4,9-tetrahydro-1H-carbazole-8-carboxylic acid, 2-hexyl-9-[(4-methylphenyl)methyl]-2,3,4,9-tetrahydro-1H-carbazole-8-carboxylic acid, 9-[(4-cyanophenyl)methyl]-2-hexyl-2,3,4,9-tetrahydro-1H-carbazole-8-carboxylic acid, 9-[(2-cyanophenyl)methyl]-2-hexyl-2,3,4,9-tetrahydro-1H-carbazole-8-carboxylic acid, 2-hexyl-9-[(2-methylphenyl)methyl]-2,3,4,9-tetrahydro-1H-carbazole-8-carboxylic acid, 9-[(2-fluorophenyl)methyl]-2-hexyl-2,3,4,9-tetrahydro-1H-carbazole-8-carboxylic acid, 9-[(3-carbamoylphenyl)methyl]-2-(2-phenylethyl)-2,3,4,9-tetrahydro-1H-carbazole-8-carboxylic acid, 9-[(3-cyanophenyl)methyl]-2-(2-phenylethyl)-2,3,4,9-tetrahydro-1H-carbazole-8-carboxylic acid, 9-[(3-carbamoylphenyl)methyl]-4-(2-phenylethyl)-2,3,4,9-tetrahydro-1H-carbazole-8-carboxylic acid, 9-[(3-cyanophenyl)methyl]-4-(2-phenylethyl)-2,3,4,9-tetrahydro-1H-carbazole-8-carboxylic acid, 9-[(3-carbamoylphenyl)methyl]-2-propyl-2,3,4,9-tetrahydro-1H-carbazole-8-carboxylic acid, 9-[(3-cyanophenyl)methyl]-2-propyl-2,3,4,9-tetrahydro-1H-carbazole-8-carboxylic acid, 2-butyl-9-[(3-carbamoylphenyl)methyl]-2,3,4,9-tetrahydro-1H-carbazole-8-carboxylic acid, 9-[(3-carbamoylphenyl)methyl]-2-pentyl-2,3,4,9-tetrahydro-1H-carbazole-8-carboxylic acid, 9-[(3-cyanophenyl)methyl]-2-pentyl-2,3,4,9-tetrahydro-1H-carbazole-8-carboxylic acid, 1-butyl-9-[(3-carbamoylphenyl)methyl]-2,3,4,9-tetrahydro-1H-carbazole-8-carboxylic acid, 9-[(3-carbamoylphenyl)methyl]-1-(pentyloxy)-2,3,4,9-tetrahydro-1H-carbazole-8-carboxylic acid, 9-[(3-cyanophenyl)methyl]-1-(pentyloxy)-2,3,4,9-tetrahydro-1H-carbazole-8-carboxylic acid, 1-butyl-9-[(3-cyanophenyl)methyl]-2,3,4,9-tetrahydro-1H-carbazole-8-carboxylic acid, 6-butyl-5-[(3-carbamoylphenyl)methyl]-5H,6H,7H,8H,9H,10H-cyclohepta[b]indole-4-carboxylic acid, 6-butyl-5-[(3-cyanophenyl)methyl]-5H,6H,7H,8H,9H,10H-cyclohepta[b]indole-4-carboxylic acid, 9-[(3-carbamoylphenyl)methyl]-1-ethyl-2,3,4,9-tetrahydro-1H-carbazole-8-carboxylic acid, 9-[(3-cyanophenyl)methyl]-1-ethyl-2,3,4,9-tetrahydro-1H-carbazole-8-carboxylic acid, 9-[(3-carboxyphenyl)methyl]-1-ethyl-2,3,4,9-tetrahydro-1H-carbazole-8-carboxylic acid, 9-[(3-carbamoylphenyl)methyl]-1-propoxy-2,3,4,9-tetrahydro-1H-carbazole-8-carboxylic acid, 9-[(3-carbamoylphenyl)methyl]-4-ethyl-2,3,4,9-tetrahydro-1H-carbazole-8-carboxylic acid, 4-[(3-carbamoylphenyl)methyl]-3-ethyl-1H,2H,3H,4H-cyclopenta[b]indole-5-carboxylic acid, 4-[(3-cyanophenyl)methyl]-3-ethyl-1H,2H,3H,4H-cyclopenta[b]indole-5-carboxylic acid, 3-butyl-4-[(3-carbamoylphenyl)methyl]-1H,2H,3H,4H-cyclopenta[b]indole-5-carboxylic acid, 3-butyl-4-[(3-cyanophenyl)methyl]-1H,2H,3H,4H-cyclopenta[b]indole-5-carboxylic acid, 2-butyl-4-[(3-carbamoylphenyl)methyl]-1H,2H,3H,4H-cyclopenta[b]indole-5-carboxylic acid, 2-butyl-4-[(3-cyanophenyl)methyl]-1H,2H,3H,4H-cyclopenta[b]indole-5-carboxylic acid, 5-[(3-carbamoylphenyl)methyl]-10-ethyl-5H,6H,7H,8H,9H,10H-cyclohepta[b]indole-4-carboxylic acid, 5-[(3-cyanophenyl)methyl]-10-ethyl-5H,6H,7H,8H,9H,10H-cyclohepta[b]indole-4-carboxylic acid, 5-[(3-carbamoylphenyl)methyl]-10-propyl-5H,6H,7H,8H,9H,10H-cyclohepta[b]indole-4-carboxylic acid, 5-[(3-cyanophenyl)methyl]-10-propyl-5H,6H,7H,8H,9H,10H-cyclohepta[b]indole-4-carboxylic acid, 9-[(3-carboxyphenyl)methyl]-4-ethyl-2,3,4,9-tetrahydro-1H-carbazole-8-carboxylic acid, 9-[(3-carbamoylphenyl)methyl]-3-ethyl-2,3,4,9-tetrahydro-1H-carbazole-8-carboxylic acid, 10-butyl-5-[(3-carbamoylphenyl)methyl]-5H,6H,7H,8H,9H,10H-cyclohepta[b]indole-4-carboxylic acid, 10-butyl-5-[(3-cyanophenyl)methyl]-5H,6H,7H,8H,9H,10H-cyclohepta[b]indole-4-carboxylic acid, 5-[(3-carbamoylphenyl)methyl]-10-pentyl-5H,6H,7H,8H,9H,10H-cyclohepta[b]indole-4-carboxylic acid, 5-[(3-cyanophenyl)methyl]-10-pentyl-5H,6H,7H,8H,9H,10H-cyclohepta[b]indole-4-carboxylic acid, 4-[(3-carbamoylphenyl)methyl]-2-pentyl-1H,2H,3H,4H-cyclopenta[b]indole-5-carboxylic acid, 4-[(3-cyanophenyl)methyl]-2-pentyl-1H,2H,3H,4H-cyclopenta[b]indole-5-carboxylic acid, 9-[(3-carbamoylphenyl)methyl]-2-ethyl-2,3,4,9-tetrahydro-1H-carbazole-8-carboxylic acid, 9-[(3-carboxyphenyl)methyl]-2-ethyl-2,3,4,9-tetrahydro-1H-carbazole-8-carboxylic acid, 5-[(3-carbamoylphenyl)methyl]-7-ethyl-5H,6H,7H,8H,9H,10H-cyclohepta[b]indole-4-carboxylic acid, 5-[(3-cyanophenyl)methyl]-7-ethyl-5H,6H,7H,8H,9H,10H-cyclohepta[b]indole-4-carboxylic acid, 9-[(3-cyanophenyl)methyl]-3-ethyl-2,3,4,9-tetrahydro-1H-carbazole-8-carboxylic acid, 9-[(3-carbamoylphenyl)methyl]-4-propyl-2,3,4,9-tetrahydro-1H-carbazole-8-carboxylic acid, 9-[(3-carbamoylphenyl)methyl]-3-propyl-2,3,4,9-tetrahydro-1H-carbazole-8-carboxylic acid, 9-[(3-cyanophenyl)methyl]-3-propyl-2,3,4,9-tetrahydro-1H-carbazole-8-carboxylic acid, 4-butyl-9-[(3-carbamoylphenyl)methyl]-2,3,4,9-tetrahydro-1H-carbazole-8-carboxylic acid, 4-butyl-9-[(3-cyanophenyl)methyl]-2,3,4,9-tetrahydro-1H-carbazole-8-carboxylic acid, 3-butyl-9-[(3-carbamoylphenyl)methyl]-2,3,4,9-tetrahydro-1H-carbazole-8-carboxylic acid, 3-butyl-9-[(3-cyanophenyl)methyl]-2,3,4,9-tetrahydro-1H-carbazole-8-carboxylic acid, 9-[(3-carbamoylphenyl)methyl]-4-pentyl-2,3,4,9-tetrahydro-1H-carbazole-8-carboxylic acid, 9-[(3-cyanophenyl)methyl]-4-pentyl-2,3,4,9-tetrahydro-1H-carbazole-8-carboxylic acid, 9-[(3-carbamoylphenyl)methyl]-3-pentyl-2,3,4,9-tetrahydro-1H-carbazole-8-carboxylic acid, 9-[(3-cyanophenyl)methyl]-3-pentyl-2,3,4,9-tetrahydro-1H-carbazole-8-carboxylic acid, 4-[(3-carbamoylphenyl)methyl]-3-propyl-1H,2H,3H,4H-cyclopenta[b]indole-5-carboxylic acid, 4-[(3-cyanophenyl)methyl]-3-propyl-1H,2H,3H,4H-cyclopenta[b]indole-5-carboxylic acid, 2-({7-butyl-5-[(3-carbamoylphenyl)methyl]-5H,6H,7H,8H,9H,10H-cyclohepta[b]indol-4-yl}formamido)acetic acid, 2-({7-butyl-5-[(3-cyanophenyl)methyl]-5H,6H,7H,8H,9H,10H-cyclohepta[b]indol-4-yl}formamido)acetic acid, 7-butyl-5-[(3-carbamoylphenyl)methyl]-N-(2-hydroxyethyl)-5H,6H,7H,8H,9H,10H-cyclohepta[b]indole-4-carboxamide, 7-butyl-5-[(3-cyanophenyl)methyl]-N-(2-hydroxyethyl)-5H,6H,7H,8H,9H,10H-cyclohepta[b]indole-4-carboxamide, 7-butyl-5-[(3-fluorophenyl)methyl]-5H,6H,7H,8H,9H,10H-cyclohepta[b]indole-4-carboxylic acid, and 7-butyl-5-[(3-carboxyphenyl)methyl]-5H,6H,7H,8H,9H,10H-cyclohepta[b]indole-4-carboxylic acid, or pharmaceutically acceptable salts or stereoisomers thereof.

Yet in other embodiments the invention is a method for treating a viral disease in a subject comprising administering to said subject a therapeutically effective dose of a compound selected from the group consisting of: 2-((2′-(5-Ethyl-3,4-diphenyl-1H-pyrazol-1-yl)-[1,1′-biphenyl]-3-yl)oxy)acetic acid, 6-chloro-4-phenyl-2-(piperidin-1-yl)-3-(1H-tetrazol-5-yl)quinoline, 5-((3-chloro-2-methylphenoxy)methyl)-2-phenyl-[1,2,4]triazolo[1,5-a]pyrimidin-7(4H)-one, and 3-(5-cyclopropyl-3-(3,5-dimethyl-1H-pyrazol-4-yl)-2-(3-isopropoxyphenyl)-1H-indol-1-yl)propanoic acid, or pharmaceutically acceptable salts or stereoisomers thereof. It should be appreciated that WO 00/59506, U.S. 2013/0116234 A1, WO 2010/056631, PCT/US21/39470, U.S. 2013/0116231 A1, Sulsky et al., Bioorg Med Chem Lett. 2007 Jun. 15; 17(12):3511-5. doi: 10.1016/j.bmc.2006.12.044. Epub 2006 Dec. 21, Kuhne et al., Bioorg Med Chem Lett. 2016 Oct. 15; 26(20):5092-5097. doi: 10.1016/j.bmcl.2016.08.071. Epub 2016 Aug. 22.), and Lan et al., J Lipid Res. 2011 April; 52(4):646-56. doi: 10.1194/jlr.M012757. Epub 2011 Feb. 4.), Tagami et al., ACS Med Chem Lett. 2016 Apr. 14; 7(4): 435-439. doi: 10.1021/acsmedchemlett.6b00040) are hereby incorporated by reference in their entirety.

The invention also describes a method for treating a viral disease in a subject comprising administering to said subject a therapeutically effective dose of one or more of the compounds described in Formula (V):

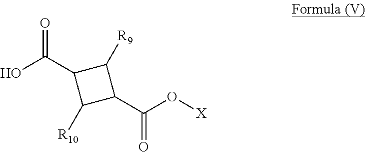

Wherein:

R₉ and R₁₀ are each independently a phenyl or anisole; and

X comprises naphthalene or fluorene;

or pharmaceutically acceptable salts or stereoisomers thereof.

The invention also describes a method for treating a viral disease in a subject comprising administering to said subject a therapeutically effective dose of one or more of the compounds described in Formula (VI):

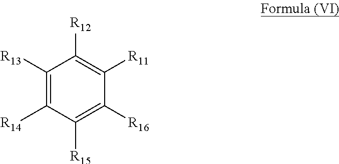

Wherein:

R₁₁ is —C(CH₃)₂ or

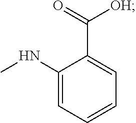

R₁₂ is —H, —O, a halogen, a phenyl, or piperidine;

R₁₃ is —C(CH₃)₂, a halogen, or

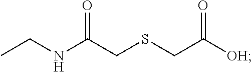

R₁₄ is a halogen, —CF₃, —NHCH₃, coumaran, or

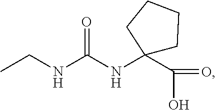

R₁₅ is —C(CH₃)₂ or a phenyl; and

R₁₆ is —COOH, a halogen, —SO₃H,

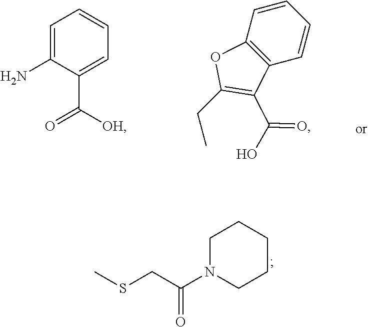

or pharmaceutically acceptable salts or stereoisomers thereof.

The invention describes a method for treating a viral disease in a subject comprising administering to said subject a therapeutically effective dose of one or more of the compounds described in Formula (VII):

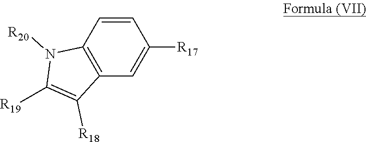

Wherein:

R₁₇ is cycloalkane;

R₁₈ is

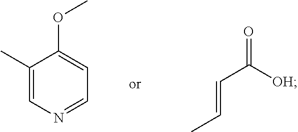

R₁₉ is a phenyl; and

R₂₀ is —(CH₂)₂COOH or

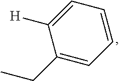

wherein H is a halogen;

or pharmaceutically acceptable salts or stereoisomers thereof.

The invention further describes a method for treating a viral disease in a subject comprising administering to said subject a therapeutically effective dose of one or more of the compounds described in Formula (VIII):

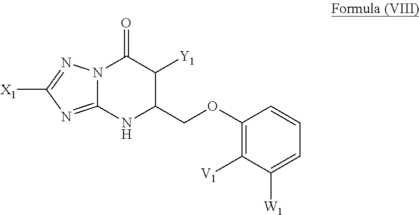

Wherein:

X₁ is a phenyl;

Y₁ and W₁ are independently a halogen; and

V₁ is —CH₃ or cycloalkane;

or pharmaceutically acceptable salts or stereoisomers thereof.

The invention describes a method for treating a viral disease in a subject comprising administering to said subject a therapeutically effective dose of one or more of the compounds described in Formula (IX):

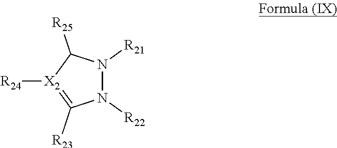

Wherein:

X₂ is —CH or O;

R₂₁ is —H or

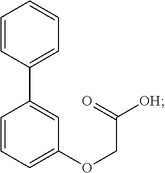 [SMILES: O=C(O)COc1cccc(-c2ccccc2)c1]

R₂₂ is —H, —O, a phenyl, or

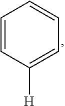

wherein H is a halogen;

R₂₃ is —H, thiophene, a phenyl

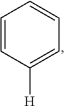

wherein H is a halogen; and

R₂₄ is —H or a phenyl; and

R₂₅ is —CH₂CH₃,

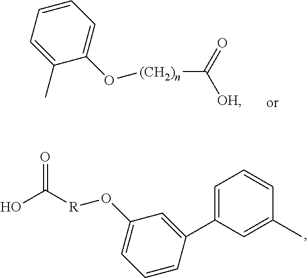

wherein n is 3 or 4, and wherein R is —CH₂ or —C(CH₃)₂;

or pharmaceutically acceptable salts or stereoisomers thereof.

The invention describes a method for treating a viral disease in a subject comprising administering to said subject a therapeutically effective dose of a compound selected from the group consisting of: 2-((2′-(5-ethyl-3,4-diphenyl-1H-pyrazol-1-yl)-[1,1′-biphenyl]-3-yl)oxy)acetic acid, 2-((2-oxo-2-((2-(piperidin-1-yl)benzyl)amino)ethyl)thio)acetic acid, (3,5-dibromo-4-hydroxyphenyl)(2-ethylbenzofuran-3-yl)methanone, (E)-4-((5-(methoxycarbonyl)-[2,2′-bithiophen]-3-yl)amino)-4-oxobut-2-enoic acid, 2-(1-(methoxymethyl)cyclopentyl)-6-pentyl-4-phenyl-3-(2H-tetrazol-5-yl)-5,6,7,8-tetrahydroquinoline, 2-cyclohexyl-4-(2-methylpyridin-4-yl)-3-(2H-tetrazol-5-yl)-6-(trifluoromethyl)-5,6,7,8-tetrahydroquinoline, 6-chloro-4-phenyl-2-(piperidin-1-yl)-3-(2H-tetrazol-5-yl)quinoline, 6-chloro-2-methyl-4-phenylquinoline-3-carboxylic acid, N-(2-(4-(1-allyl-2,4-dioxo-2,3,4,5-tetrahydro-1H-pyrrolo[3,2-d]pyrimidin-6-yl)phenoxy)ethyl)picolinamide, (5-chloro-3-hydroxyquinoxalin-2-yl)(2-(3,5-dichlorobenzyl)pyrrolidin-1-yl)methanone, 1-(3-(4,6-dichloro-[1,1′-biphenyl]-2-yl)ureido)cyclopentane-1-carboxylic acid, 5-((3-chloro-2-methylphenoxy)methyl)-2-phenyl-[1,2,4]triazolo[1,5-a]pyrimidin-7(4H)-one, 6-chloro-5-((3-chloro-2-cyclopropylphenoxy)methyl)-2-phenyl-[1,2,4]triazolo[1,5-a]pyrimidin-7(4H)-one, 2-((3-chloro-2-(2,3-dihydrobenzofuran-5-yl)phenyl)amino)benzoic acid, 2-((6-chloro-5-(methylamino)-[1,1′-biphenyl]-2-yl)amino)benzoic acid, (1S,2R,3S,4R)-2,4-bis(2-methoxyphenyl)-3-((naphthalen-1-yloxy)carbonyl)cyclobutane-1-carboxylic acid, (1R,2R,3R,4R)-3-(((9H-fluoren-9-yl)methoxy)carbonyl)-2,4-bis(2-methoxyphenyl)cyclobutane-1-carboxylic acid, (1R,2R,3R,4R)-3-((naphthalen-1-yloxy)carbonyl)-2,4-diphenylcyclobutane-1-carboxylic acid, 1-[l-[4,4-Bis(4-fluorophenyl)butyl]-4-piperidinyl]-1,3-dihydro-2H-benzimidazole-2-one, 2,4,6-triisopropylbenzoic acid, 2,4,6-triisopropylbenzenesulfonic acid, 2-((2′-(1-(4-chlorophenyl)-5-(thiophen-2-yl)-1H-pyrazol-3-yl)-[1,1′-biphenyl]-3-yl)oxy)-2-methylpropanoic acid, (E)-3-(1-(2-fluorobenzyl)-1H-indol-3-yl)acrylic acid, 4-(2-(5-(2-chlorophenyl)-1-phenyl-1H-pyrazol-3-yl)phenoxy)butanoic acid, 5-(2-(4,5-diphenyloxazol-2-yl)phenoxy)pentanoic acid, 2-((2′-(4,5-diphenyloxazol-2-yl)-[1,1′-biphenyl]-3-yl)oxy)acetic acid, 4-(9H-carbazol-9-yl)butanoic acid, 3-((9H-carbazol-9-yl)sulfonyl)thiophene-2-carboxylic acid, 5-(3-carbamoylbenzyl)-5,6,7,8,9,10-hexahydrocyclohepta[b]indole-4-carboxylic acid, 9-(2-(trifluoromethyl)benzyl)-2,3,4,9-tetrahydro-1H-carbazole-8-carboxylic acid, 3-(5-cyclopropyl-3-(3,5-dimethyl-1H-pyrazol-4-yl)-2-(3-isopropoxyphenyl)-1H-indol-1-yl)propanoic acid, 3-(5-cyclopropyl-3-(3-methoxypyridin-4-yl)-2-phenyl-1H-indol-1-yl)propanoic acid, and 2-((2-oxo-2-(piperidin-1-yl)ethyl)thio)-6-(trifluoromethyl)pyrimidin-4(3H)-one.

The present invention also describes a method for treating a viral disease in a subject comprising administering to said subject a therapeutically effective dose of one or more of the compounds described in Formula X:

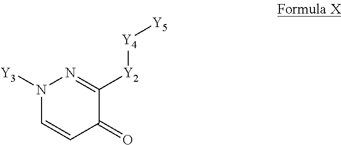

Wherein:

Y₂ is CR¹³R¹³, 0, NR¹⁵ or S;

Y₃ is heteroaryl or aryl, wherein said heteroaryl and aryl groups are optionally substituted with one to three groups independently selected from the group consisting of halo, cyano, C₁₋₆ alkyl, (C₁₋₆ alkyl)R¹⁷, OR¹⁷, (C=0)NR¹⁵R¹⁶, heterocyclyl, aryl, heteroaryl and (heteroaryl)R¹⁶;

Y₄ is CR¹⁴R¹⁴0, C₂₋₃ alkenyl, 0, NR¹⁵ or S;

Y₅ is heteroaryl or phenyl, wherein said heteroaryl and phenyl groups are optionally substituted with one to three groups independently selected from the group consisting of halo, cyano, oxo, C₁₋₆ alkyl, (C₁₋₆ alkyl)0R¹⁶, 0R¹⁶, R¹⁷, 0R¹⁷, 0(C₁₋₆ alkyl)0R¹⁶, 0(C₁₋₆ alkyl)R¹⁷, (C=0)R¹⁶, (C=0)0R¹⁶, (C=0)NHR¹⁶, (C=0)R¹⁷, NHR¹⁶, NH(C0)0R¹⁶, NH(C=0)R¹⁷, NH(C=0)0, (C₁₋₆ alkyl)0R¹⁶ and N0₂;

R¹³ is hydrogen, halo or C₁₋₆ alkyl;

R¹⁴ is hydrogen, halo, C₁₋₆ alkyl or (C=0)0R¹⁶;

R¹⁵ is hydrogen or C₁₋₆ alkyl,

R¹⁶ is hydrogen or C₁₋₆ alkyl, wherein said alkyl is optionally substituted with one to three groups independently selected from the group consisting of halo and hydroxyl; R¹⁷ is hydrogen, heterocyclyl, aryl or heteroaryl, wherein said heterocyclyl and heteroaryl groups are optionally substituted with one to two groups independently selected from the group consisting of cyano, halo, hydroxyl, R¹⁶, R¹⁸, 0R¹⁶, (C₁₋₆ alkyl)0R¹⁶, (C₁₋₆ alkyl)0R¹⁸, S0iC₁₋₆ alkyl), (C=0)R¹⁸; and

R¹⁸ is heterocyclyl or heteroaryl, wherein said heterocyclyl group is optionally substituted with cyano, halo, hydroxyl, R¹⁶, 0R¹⁶ or (C=0)0R¹⁶;

or a pharmaceutically acceptable salt thereof.

The present invention also describes a method for treating a viral disease in a subject comprising administering to said subject a therapeutically effective dose of one or more of the compounds described in Formula (XI):

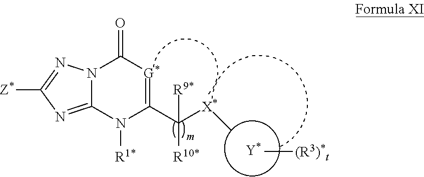

wherein:

Y* is a ring as described below;

X* is either:

(i) C(R>7′oR8^{α}), N(FT), S, S(O₂), or O, and both

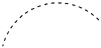

are absent; or

(ii) C(R⁷), and X* is linked, either to the ring marked Y* as shown by the

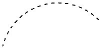

towards the right of X*, or to the position marked G* on the triazolopyrimidinone ring as shown by the

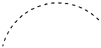

towards the left of X*, to form a 3-8 membered cycloalkyl or a 4-8 membered heterocyclyl ring containing 1-3 heteroatoms independently selected from N, O and S, or (iii) N (with R³ being absent), and X* is linked, either to the ring marked Y* as shown by the

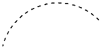

towards the right of X*, or to the position marked G* on the triazolopyrimidinone ring as shown by the

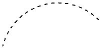

towards the left of X*, to form a 4-8 membered heterocyclyl ring containing 1-3 heteroatoms independently selected from N, O and S, including the nitrogen atom of X*; further wherein

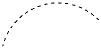

shown to the right of X* is mutually exclusive of

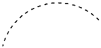

shown to the left of X*;

G* is C(R⁶) when

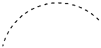

shown to the left of X* is absent, and G* is C when

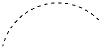

shown to the left of X* is present;

Ring Y* is aryl, heteroaryl, heterocyclyl or cycloalkyl, and when

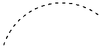

shown to the right of X* is present, a ring atom on Y* is the point of attachment for said

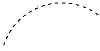

shown to the right of X*, wherein each of said aryl, heteroaryl, heterocyclyl and cycloalkyl can be unsubstituted or optionally independently substituted with one or more substituents which can be the same or different, each substituent being independently selected from the group consisting of halo, hydroxyl, alkyl, alkoxy, CN and CF₃;

R₃ may be present or absent as stated above, and when R³ is present, R³ is H, alkyl or cycloalkyl, wherein each of said alkyl and cycloalkyl may be unsubstituted or optionally independently substituted with one or more substituents which can be the same or different, each substituent being independently selected from the group consisting of halo, hydroxyl, alkoxy, CN and CF₃;

R¹* is H, alkyl, -alkyl-OR⁴, haloalkyl, haloalkoxy or -alkyl-CN; m is 1-2; t is 0-4; each

R²* is independently selected from the group consisting of halo, CN, —OR⁵, alkyl, haloalkyl, haloalkoxy, aryl, heteroaryl, heterocyclyl, cycloalkyl, —NH₂, —NH(alkyl), —NH(aryl), —NH(heteroaryl), —NH(cycloalkyl), —S-alkyl, —S-aryl, —S-heteroaryl, —S-cycloalkyl, —C(O)OH, —C(O)—NH₂, —C(O)N(H)(alkyl), —C(O)N(H)aryl, —C(O)N(H)(heteroaryl), —C(O)N(H)(heterocyclyl), —C(O)—N(H)(cycloalkyl), —C(O)N(alkyl)₂, —C(O)N(aryl)₂, —C(O)N(heteroaryl)₂, —C(O)N(heterocyclyl)₂, —C(O)—N(cycloalkyl)₂, —C(O)N(aryl)(alkyl), —C(O)N(heteroaryl)(aryl), —C(O)N(heterocyclyl)(heteroaryl), —C(O)N(aryl)(heterocyclyl), —C(O)—N(alkyl)(cycloalkyl), —C(O)N(cycloalkyl)(aryl), —C(O)N(cycloalkyl)(heterocyclyl), —NH—C(O)—NH₂, —C(O)R⁵, and —C(O)OR⁵, wherein each of said alkyl, aryl, heteroaryl, heterocyclyl and cycloalkyl may be unsubstituted or optionally independently substituted with one or more substituents which can be the same or different, each substituent being independently selected from the group consisting of halo, alkyl, aryl, heteroaryl, hydroxyl, cycloalkyl, alkoxy, —C(O)R⁵, CN and CF₃;

R* is H, —OR⁵, —C(O)OR⁵, alkyl, alkenyl, alkynyl, cycloalkyl, haloalkyl, aryl or heteroaryl, wherein each of said alkyl, cycloalkyl, haloalkyl, aryl and heteroaryl may be unsubstituted or optionally independently substituted with one or more substituents which can be the same or different, each substituent being independently selected from the group consisting of halo, CN, —OR⁵, alkyl, haloalkyl, haloalkoxy, aryl, heteroaryl, heterocyclyl, cycloalkyl, —C(O)—NH₂, —C(O)N(H)(CH₃), —NH—C(O)—NH₂, —C(O)R⁵, and —C(O)OR⁵;

R⁴ is H, alkyl, aryl or heteroaryl, wherein each of said alkyl, aryl and heteroaryl may be unsubstituted or optionally independently substituted with one or more substituents which can be the same or different, each substituents being independently selected from the group consisting of halo, hydroxyl, alkoxy, CN and CF₃;

R⁵ is H, alkyl, cycloalkyl, aryl or heteroaryl, wherein each of said alkyl, cycloalkyl, aryl and heteroaryl may be unsubstituted or optionally independently substituted with one or more substituents which can be the same or different, each substituents being independently selected from the group consisting of halo, hydroxyl, alkoxy, —OC(O)R⁴, CN and CF₃; R⁶ is H, halo, alkyl, aryl or heteroaryl, wherein each of said alkyl, aryl and heteroaryl may be unsubstituted or optionally independently substituted with one or more substituents which can be the same or different, each substituents being independently selected from the group consisting of halo, hydroxyl, alkoxy, CN and CF₃; R⁷ is H, alkyi, hydroxy or alkoxy, wherein each of said alkyl and alkoxy may be independently unsubstituted or optionally independently substituted with one or more substituents which can be the same or different, each substituent being independently selected from the group consisting of halo, hydroxyl, alkoxy, CN and CF₃;

R⁸ is H, alkyl, hydroxy or alkoxy, wherein each of said alkyl and alkoxy may be independently unsubstituted or optionally independently substituted with one or more substituents which can be the same or different, each substituent being independently selected from the group consisting of halo, hydroxyl, alkoxy, CN and CF₃;

R⁹* is H, halo, alkyl, cycloalkyl, aryl or heteroaryl, wherein each of said alkyl, aryl and heteroaryl may be unsubstituted or optionally independently substituted with one or more substituents which can be the same or different, each substituents being independently selected from the group consisting of halo, hydroxyl, alkoxy, CN and CF₃; and

R¹⁰* is H, halo, alkyl, cycloalkyl, aryl or heteroaryl, —C(O)OH, —C(O)—NH₂, —C(O)N(H)(alkyl), —C(O)N(H)aryl, —C(O)N(H)(heteroaryl), —C(O)N(H)(heterocyclyl), —C(O)—N(H)(cycloalkyl), —C(O)N(alkyl)₂, —C(O)N(aryl)₂, —C(O)N(heteroaryl)₂, —C(O)N(heterocyclyl)₂, —C(O)—N(cycloalkyl)₂, —C(O)N(aryl)(alkyl), —C(O)N(heteroaryl)(aryl), —C(O)N(heterocyclyl)(heteroaryl), —C(O)N(aryl)(heterocyclyl), —C(O)—N(alkyl)(cycloalkyl), —C(O)N(cycloalkyl)(aryl), —C(O)N(cycloalkyl)(heterocyclyl), —NH—C(O)—NH₂, —C(O)R⁵, and —C(O)OR⁵, wherein each of said alkyl, aryl, heteroaryl, heterocyclyl and cycloalkyl may be unsubstituted or optionally independently substituted with one or more substituents which can be the same or different, each substituent being independently selected from the group consisting of halo, alkyl, aryl, heteroaryl, hydroxyl, cycloalkyl, alkoxy, —C(O)R⁵, CN and CF₃; or alternatively,

R⁹* and R₁₀* can together form ═O or R⁹* and R¹⁰* can be joined to form a spirocyclyl group.

The present invention also describes a method for treating a viral disease in a subject comprising administering to said subject a therapeutically effective dose of one or more of the compounds described in Formula (XII):

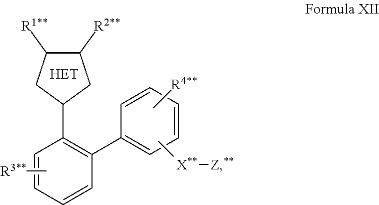

its stereoisomers, a pharmaceutically acceptable salt or prodrug ester thereof,

wherein R¹** and R²** are the same or different and are independently selected from, alkyl, cycloalkyl, cycloalkenyl, aryl, aralkyl, or;

R³** is selected from hydrogen, halogen, alky, alkenyl, alkynyl, alkoxy, cycloalkyl, cycloalkylalkyl, cycloalkenyl, alkylcarbonyl, cycloalkenylalkyl, haloalkyl, polyhaloalkyl, cyano, nitro, hydroxy, amino, alkanoyl, alkylthio, alkylsulfonyl, alkoxycarbonyl, alkylaminocarbonyl, alkylcarbonylamino, alkylcarbonyloxy, alkylaminosulfonyl, alkylamino, dialkylamino, all optionally substituted through available carbon atoms with 1, 2, 3, 4 or 5 groups selected from hydrogen, halo, alkyl, polyhaloalkyl, alkoxy, haloalkoxy, polyhaloalkoxy, alkoxycarbonyl, alkenyl, alkynyl, cycloalkyl, cycloalkylalkyl, hydroxy, hydroxyalkyl, nitro, cyano, amino, substituted amino, alkylamino, dialkylamino, thiol, alkylthio, alkylcarbonyl, acyl, alkoxycarbonyl, aminocarbonyl, alkynylaminocarbonyl, alkylaminocarbonyl, alkenylaminocarbonyl, alkylcarbonyloxy, alkylcarbonylamino, arylcarbonylamino, alkoxycarbonylamino, alkylsulfonyl, aminosulfinyl, aniinosulfonyl, alkylsulfinyl, sulfonamido or sulfonyl;

R⁴** is selected from hydrogen, halogen, alkyl, alkenyl, alkynyl, alkoxy, aryl, arylalkyl, arylalkenyl, arylalkynyl, cycloalkyl, cycloalkylalkyl, polycycloalkyl, polycycloalkylaikyl, cycloalkenyl, cycloalkynyl, alkylcarbonyl, arylcarbonyl, cycloheteroalkyl, cycooheteroalkylalkyl, cycloalkenylalkyl, polycycloalkenyl, polycycloalkenylalkyl, polycycloalkynyl, polveycloalkynylalkyl, haloalkyl, polyhaloalkyl, cyano, nitro, hydroxy, amino, alkanoyl, aroyl, alkylsulfonyl, arylsulfonyl, alkoxycarbonyl, aryloxycarbonyl, alkylaminocarbonyl, arylaminocarbonyl, alkylcarbonylamino, alkoxycarbonyloxy, alkylaminosulfonyl, arylaminosulfonyl, alkylamino, dialkylamino, all optionally substituted through available carbon atoms with 1, 2, 3, 4 or 5 groups selected from hydrogen, halo, haloalkyl, polyhaloalkyl, alkoxy, haloalkoxy, polyhaloalkoxy, alkoxycarbonyl, alkenyl, alkynyl, cycloalkyl, cycloalkylalkyl, aryl, arylalkyl, arylcycloalkyl, arylalkenyl, arylalkynyl, aryloxy, aryloxyalkyl, arylalkoxy, arylazo, hydroxy, hydroxyalkyl, nitro, cyano, amino, substituted amino, alkylamino, dialkylamino, thiol, alkylthio, arylthio, arylthioalkyl, alkylcarbonyl, arylcarbonyl, acyl, arylaminocarbonyl, alkoxycarbonyl, aminocarbonyl, alkynylaminocarbonyl, alkylaminocarbonyl, alkenylaminocarbonyl, alkylcarbonyloxy, arylcarbonyloxy, alkylcarbonylamino, arylcarbonylamino, alkoxycarbonylamino, arylsulfinyl, arylsulfmylalkyl, arylsulfonyl, alkylsulfonyl, aminosulfinyl, aminosulfonyl, arylsulfonylamino, alkylsulfinyl, sulfonamido or sulfonyl;

X** is a bond or a linker group selected from (CH₂)ₙ, O(CH₂)ₙ, S(CH₂)ₙ, cycloalkylene, N(R⁵**)(CH₂)ₙ, NHCO, or CH═CH where n=0-5 and R⁵ is hydrogen alkyl, or alkanoyl;

Z* is CO₂H or tetrazole of the formula

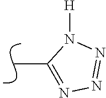

or its tautomer; and the group

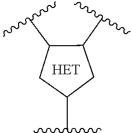 [SMILES: *C1CC(*)C(*)C1]

represents a heteroaryl group of the formula

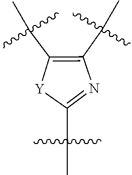

where Y is NR⁸** or O;

which may further be optionally substituted with one or two groups, which may be the same or different and are independently selected from alkyl, alkenyl, hydroxyalkyl, keto, carboxyalkyl, carboxy, cycloalkyl, alkoxy, formyl, alkanoyl, alkoxyalkyl or alkoxycarbonyl, with the provisos that

(1) n≠0 when Z*8 is CO₂H and X** is O(CH₂)ₙ, S(CH₂), or N(R⁵)CH₂)ₙ); and

(2) when

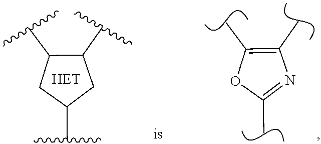

then X**—Z** may not be O-lower alkylene-CO₂H or —O-lower alkylene-CO₂alkyl when R¹** and R²** are both aryl or substituted aryl and R²** and R⁴** are each hydrogen.

It is an object of the present invention to utilize the compounds described herein in the prophylaxis or treatment of viral disorders by acting on the fatty acid binding protein (FABP4), such as common cold, SARS, and COVID-19.

Yet another object of the present invention is a pharmaceutical composition comprising a compounds herein as active ingredient, in combination with a pharmaceutically acceptable diluent or carrier for use in the treatment of viral disorders by acting on FABP4. Here, the pharmaceutical composition can further comprise an additional therapeutically active agent.

Yet another object of the present invention is a method for the treatment of viral disorders by acting on the FABP4, which comprises administering to a subject in need of such treatment (preferably, a human) an effective amount of the compounds herein, including, optionally, the co-administration with other therapeutic agents, either as a single (or multiple) dosing, and either simultaneously or sequentially.

Any compound disclosed herein which is a pure optical isomer.

Any compound disclosed herein which is the (+)-isomer.

### Definitions

As used herein, the term “acid isostere” includes, but is not limited to, the following functional groups where R is an alkyl group:

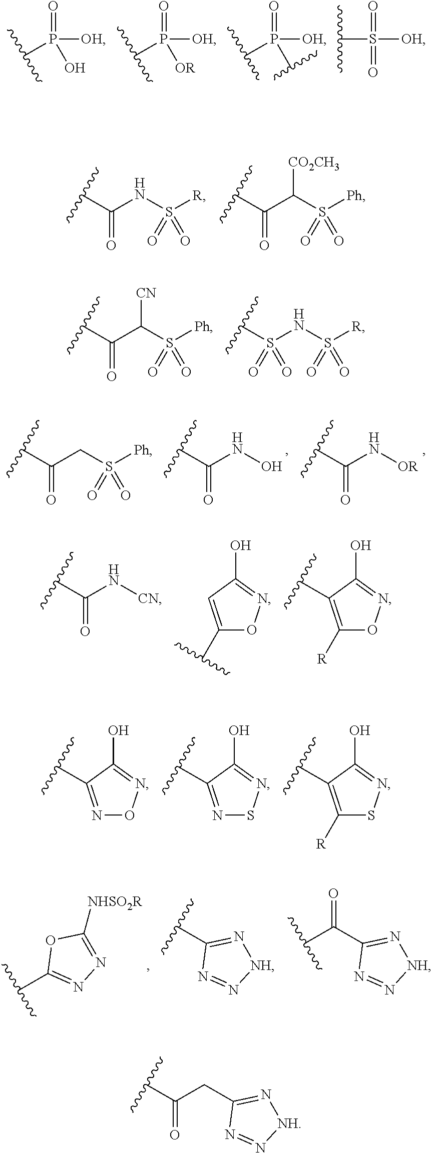

The term “alkyl” refers to a saturated, straight- or branched-chain hydrocarbon group having from 1 to 10 carbon atoms. Representative alkyl groups include, but are not limited to, methyl, ethyl, n-propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, butyl, isobutyl, t-butyl, n-pentyl, isopentyl, neopentyl, n-hexyl, and the like, and longer alkyl groups, such as heptyl, octyl, and the like. As used herein, “lower alkyl” means an alkyl having from 1 to 6 carbon atoms.

The term “alkylenyl” refers to a divalent alkyl group.

The term “alkoxy” as used herein includes —O-(alkyl), wherein alkyl is defined above.

The term “amino” as used herein refers to an —NH₂ group.

“Aryl” means a mono-, bi-, or tricyclic aromatic group, wherein all rings of the group are aromatic and all ring atoms are carbon atoms. For bi- or tricyclic systems, the individual aromatic rings are fused to one another. Examples of aryl groups are 6 and 10 membered aryls.

Further examples of aryl groups include, but are not limited to, phenyl, naphthalene, and anthracene.

The term “cyano” as used herein means a substituent having a carbon atom joined to a nitrogen atom by a triple bond.

The term “deuterium” as used herein means a stable isotope of hydrogen having one proton and one neutron.

The term “halo” represents chloro, fluoro, bromo, or iodo. In some embodiments, halo is chloro, fluoro, or bromo. The term “halogen” as used herein refers to fluorine, chlorine, bromine, or iodine.

The term “hydroxy” means an —OH group.

The term “oxo” means an ═O group and may be attached to a carbon atom or a sulfur atom.

The term “N-oxide” refers to the oxidized form of a nitrogen atom.

As used herein, the term “cycloalkyl” refers to a saturated or partially saturated, monocyclic, fused polycyclic, bridged polycyclic, or spiro polycyclic carbocycle having from 3 to 15 carbon ring atoms. A non-limiting category of cycloalkyl groups are saturated or partially saturated, monocyclic carbocycles having from 3 to 6 carbon atoms. Illustrative examples of cycloalkyl groups include, but are not limited to, the following moieties:

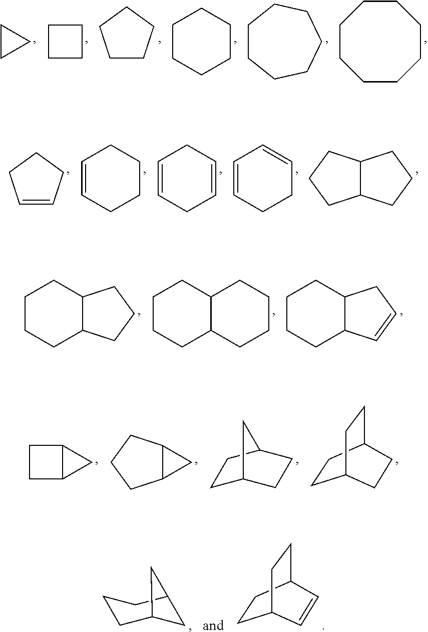

“Heterocycloalkyl” as used herein refers to a monocyclic, or fused, bridged, or spiro polycyclic ring structure that is saturated or partially saturated and has from three to 12 ring atoms selected from carbon atoms and up to three heteroatoms selected from nitrogen, oxygen, and sulfur. The ring structure may optionally contain up to two oxo groups on carbon or sulfur ring members, or an N-oxide. Illustrative heterocycloalkyl entities include, but are not limited to:

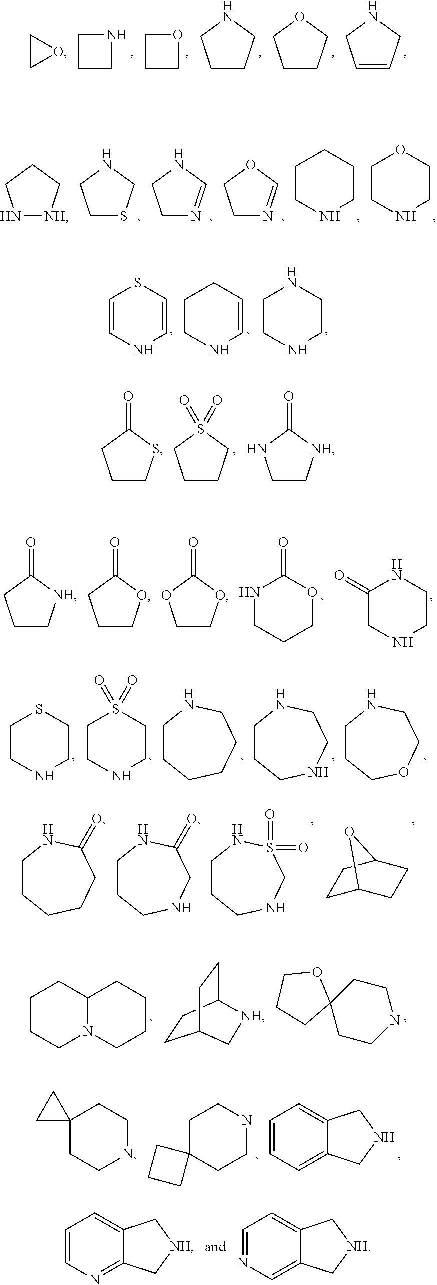

As used herein, the term “heteroaryl” refers to a monocyclic, or fused polycyclic, aromatic heterocycle having from three to 15 ring atoms that are selected from carbon, oxygen, nitrogen, and sulfur. Suitable heteroaryl groups do not include ring systems that must be charged to be aromatic, such as pyrylium. Suitable 5-membered heteroaryl rings (as a monocyclic heteroaryl or as part of a polycyclic heteroaryl) have one oxygen, sulfur, or nitrogen ring atom, or one nitrogen plus one oxygen or sulfur, or 2, 3, or 4 nitrogen ring atoms. Suitable 6-membered heteroaryl rings (as a monocyclic heteroaryl or as part of a polycyclic heteroaryl) have 1, 2, or 3 nitrogen ring atoms. Examples of heteroaryl groups include, but are not limited to, pyridinyl, imidazolyl, imidazopyridinyl, pyrimidinyl, pyrazolyl, triazolyl, pyrazinyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, triazolyl, thiadiazolyl, furazanyl, benzofurazanyl, benzothiophenyl, benzothiazolyl, benzoxazolyl, quinazolinyl, quinoxalinyl, naphthyridinyl, and furopyridinyl.

The term “bicyclic heteroaryl” refers to a heteroaryl as defined above, having two constituent aromatic rings, wherein the two rings are fused to one another and at least one of the rings is a heteroaryl as defined above. Bicyclic heteroaryls include bicyclic heteroaryl groups comprising 1, 2, 3, or 4 heteroatom ring atoms selected from O, N or S. In certain embodiments, wherein the heteroatom is N it can be an N-oxide. Bicyclic heteroaryls also include 8-, 9-, or 10-membered bicyclic heteroaryl groups. Bicyclic heteroaryls also include 8-, 9-, or 10-membered bicyclic heteroaryl groups that have 1, 2, 3, or 4 heteroatom ring atoms selected from O, N or S. Illustrative examples of bicyclic heteroaryls include, but are not limited to:

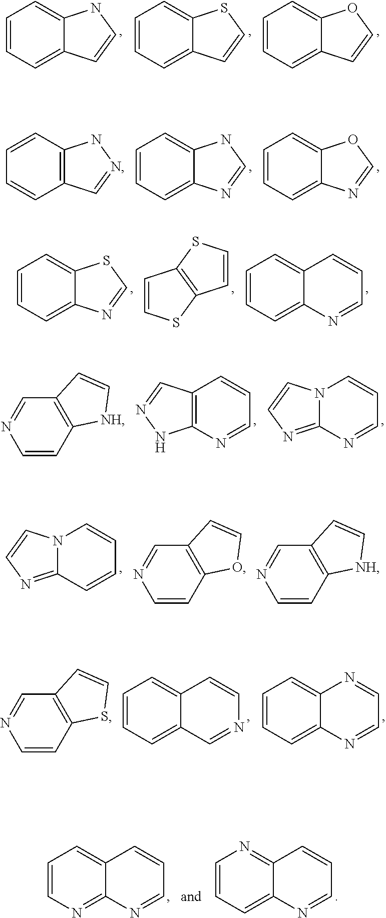

Those skilled in the art will recognize that the species of heteroaryl, cycloalkyl, and heterocycloalkyl groups listed or illustrated above are not exhaustive, and that additional species within the scope of these defined terms may also be selected.

As used herein, the term “substituted” means that the specified group or moiety bears one or more suitable substituents. As used herein, the term “unsubstituted” means that the specified group bears no substituents. As used herein, the term “optionally substituted” means that the specified group is unsubstituted or substituted by the specified number of substituents. Where the term “substituted” is used to describe a structural system, the substitution is meant to occur at any valency-allowed position on the system.

As used herein, the expression “one or more substituents” denotes one to maximum possible number of substitution(s) that can occur at any valency-allowed position on the system. In a certain embodiment, one or more substituent means 1, 2, 3, 4, or 5 substituents. In another embodiment, one or more substituent means 1, 2, or 3 substituents.

Any atom that is represented herein with an unsatisfied valence is assumed to have the sufficient number of hydrogen atoms to satisfy the atom's valence.

When any variable (e.g., alkyl or Ra) appears in more than one place in any formula or description provided herein, the definition of that variable on each occurrence is independent of its definition at every other occurrence.

Numerical ranges, as used herein, are intended to include sequential whole numbers. For example, a range expressed as “from 0 to 4” or “0-4” includes 0, 1, 2, 3 and 4.

When a multifunctional moiety is shown, the point of attachment to the remainder of the formula can be at any point on the multifunctional moiety. In some embodiments, the point of attachment is indicated by a line or hyphen. For example, aryloxy- refers to a moiety in which an oxygen atom is the point of attachment to the core molecule while aryl is attached to the oxygen atom.

### Additional Definitions

As used herein, “proton nuclear magnetic resonance” or 1H NMR is the application of nuclear magnetic resonance in NMR spectroscopy with respect to hydrogen-1 nuclei within the molecules of a substance, in order to determine the structure of its molecules.

As used herein, the term “subject” encompasses mammals and non-mammals. Examples of mammals include, but are not limited to, any member of the Mammalian class: humans; non-human primates such as chimpanzees, and other apes and monkey species; farm animals such as cattle, horses, sheep, goats, swine; domestic animals such as rabbits, dogs, and cats; and laboratory animals including rodents, such as rats, mice and guinea pigs, and the like. Examples of non-mammals include, but are not limited to, birds, fish and the like. In one embodiment of the present invention, the mammal is a human.

“Patient” includes both human and animals.

The term “inhibitor” refers to a molecule such as a compound, a drug, an enzyme activator, or a hormone that blocks or otherwise interferes with a particular biologic activity.

The term “modulator” refers to a molecule, such as a compound of the present invention, that increases or decreases, or otherwise affects the activity of a given enzyme or protein.

The terms “effective amount” or “therapeutically effective amount” refer to a sufficient amount of the agent to provide the desired biological result. That result can be reduction and/or alleviation of the signs, symptoms, or causes of a disease or medical condition, or any other desired alteration of a biological system. For example, an “effective amount” for therapeutic use is the amount of a compound, or of a composition comprising the compound, that is required to provide a clinically relevant change in a disease state, symptom, or medical condition. An appropriate “effective” amount in any individual case may be determined by one of ordinary skill in the art using routine experimentation. Thus, the expression “effective amount” generally refers to the quantity for which the active substance has a therapeutically desired effect.

As used herein, the terms “treat” or “treatment” encompass both “preventative” and “curative” treatment. “Preventative” treatment is meant to indicate a postponement of development of a disease, a symptom of a disease, or medical condition, suppressing symptoms that may appear, or reducing the risk of developing or recurrence of a disease or symptom. “Curative” treatment includes reducing the severity of or suppressing the worsening of an existing disease, symptom, or condition. Thus, treatment includes ameliorating or preventing the worsening of existing disease symptoms, preventing additional symptoms from occurring, ameliorating or preventing the underlying metabolic causes of symptoms, inhibiting the disorder or disease, e.g., arresting the development of the disorder or disease, relieving the disorder or disease, causing regression of the disorder or disease, relieving a condition caused by the disease or disorder, or stopping the symptoms of the disease or disorder.

### Additional Chemical Descriptions

Any formula given herein is intended to represent compounds having structures depicted by the structural formula as well as certain variations or forms. For example, compounds of any formula given herein may have asymmetric or chiral centers and therefore exist in different stereoisomeric forms. All stereoisomers, including optical isomers, enantiomers, and diastereomers, of the compounds of the general formula, and mixtures thereof, are considered to fall within the scope of the formula. Furthermore, certain structures may exist as geometric isomers (i.e., cis and trans isomers), as tautomers, or as atropisomers. All such isomeric forms, and mixtures thereof, are contemplated herein as part of the present invention. Thus, any formula given herein is intended to represent a racemate, one or more enantiomeric forms, one or more diastereomeric forms, one or more tautomeric or atropisomeric forms, and mixtures thereof.

Diastereomeric mixtures may be separated into their individual diastereomers on the basis of their physical chemical differences by methods well known to those skilled in the art, such as, for example, by chromatography and/or fractional crystallization. Enantiomers may be separated by converting the enantiomeric mixture into a diastereomeric mixture by reaction with an appropriate optically active compound (e.g., chiral auxiliary such as a chiral alcohol or Mosher's acid chloride, or formation of a mixture of diastereomeric salts), separating the diastereomers and converting (e.g., hydrolyzing or de-salting) the individual diastereomers to the corresponding pure enantiomers. Enantiomers may also be separated by use of chiral HPLC column. The chiral centers of compounds of the present invention may be designated as “R” or “S” as defined by the IUPAC 1974 Recommendations.

The compounds of the invention can form pharmaceutically acceptable salts, which are also within the scope of this invention. A “pharmaceutically acceptable salt” refers to a salt of a free acid or base of a compound of the invention that is non-toxic, is physiologically tolerable, is compatible with the pharmaceutical composition in which it is formulated, and is otherwise suitable for formulation and/or administration to a subject. Reference to a compound herein is understood to include reference to a pharmaceutically acceptable salt of said compound unless otherwise indicated.

Compound salts include acidic salts formed with inorganic and/or organic acids, as well as basic salts formed with inorganic and/or organic bases. In addition, where a given compound contains both a basic moiety, such as, but not limited to, a pyridine or imidazole, and an acidic moiety, such as, but not limited to, a carboxylic acid, one of skill in the art will recognize that the compound may exist as a zwitterion (“inner salt”); such salts are included within the term “salt” as used herein. Salts of the compounds of the invention may be prepared, for example, by reacting a compound with an amount of a suitable acid or base, such as an equivalent amount, in a medium such as one in which the salt precipitates or in an aqueous medium followed by lyophilization.

Exemplary salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucuronate, saccharate, formate, benzoate, glutamate, methanesulfonate (“mesylate”), ethanesulfonate, benzenesulfonate, p-toluenesulfonate, and pamoate (i.e., 1,1′-methylene-bis(2-hydroxy-3-naphthoate)) salts. A pharmaceutically acceptable salt may involve the inclusion of another molecule such as an acetate ion, a succinate ion or other counterion. The counterion may be any organic or inorganic moiety that stabilizes the charge on the parent compound. Furthermore, a pharmaceutically acceptable salt may have more than one charged atom in its structure. Instances where multiple charged atoms are part of the pharmaceutically acceptable salt can have multiple counterions. Hence, a pharmaceutically acceptable salt can have one or more charged atoms and/or one or more counter ion.

Exemplary acid addition salts include acetates, ascorbates, benzoates, benzenesulfonates, bisulfates, borates, butyrates, citrates, camphorates, camphorsulfonates, fumarates, hydrochlorides, hydrobromides, hydroiodides, lactates, maleates, methanesulfonates, naphthalenesulfonates, nitrates, oxalates, phosphates, propionates, salicylates, succinates, sulfates, tartarates, thiocyanates, toluenesulfonates (also known as tosylates) and the like.

Exemplary basic salts include ammonium salts, alkali metal salts such as sodium, lithium, and potassium salts, alkaline earth metal salts such as calcium and magnesium salts, salts with organic bases (for example, organic amines) such as dicyclohexylamines, t-butyl amines, and salts with amino acids such as arginine, lysine and the like. Basic nitrogen-containing groups may be quarternized with agents such as lower alkyl halides (e.g., methyl, ethyl, and butyl chlorides, bromides and iodides), dialkyl sulfates (e.g., dimethyl, diethyl, and dibutyl sulfates), long chain halides (e.g., decyl, lauryl, and stearyl chlorides, bromides and iodides), aralkyl halides (e.g., benzyl and phenethyl bromides), and others.

Additionally, acids and bases which are generally considered suitable for the formation of pharmaceutically useful salts from pharmaceutical compounds are discussed, for example, by P. Stahl et al., Camille G. (eds.) Handbook of Pharmaceutical Salts: Properties, Selection and Use. (2002) Zurich: Wiley-VCH; S. Berge et al., J. Pharm. Sci. (1977) 66(1) 1-19; P. Gould, Int. J. Pharm. (1986) 33 201-217; Anderson et al., The Practice of Medicinal Chemistry (1996), Academic Press, New York; and in The Orange Book (Food & Drug Administration, MD, available from FDA). These disclosures are incorporated herein by reference thereto.

Additionally, any compound described herein is intended to refer also to any unsolvated form, or a hydrate, solvate, or polymorph of such a compound, and mixtures thereof, even if such forms are not listed explicitly. “Solvate” means a physical association of a compound of the invention with one or more solvent molecules. This physical association involves varying degrees of ionic and covalent bonding, including hydrogen bonding. In certain instances, the solvate will be capable of isolation, for example when one or more solvent molecules are incorporated in the crystal lattice of a crystalline solid. “Solvate” encompasses both solution-phase and isolatable solvates. Suitable solvates include those formed with pharmaceutically acceptable solvents such as water, ethanol, and the like. In some embodiments, the solvent is water and the solvates are hydrates.

One or more compounds of the invention may optionally be converted to a solvate. Methods for the preparation of solvates are generally known. Thus, for example, M. Caira et al., J. Pharm. Sci., 93(3), 601-611 (2004), describes the preparation of the solvates of the antifungal fluconazole in ethyl acetate as well as from water. Similar preparations of solvates, hemisolvate, hydrates, and the like are described by E. C. van Tonder et al., AAPS PharmSciTech., 5(1), article 12 (2004); and A. L. Bingham et al., Chem. Commun., 603-604 (2001). A typical, non-limiting process involves dissolving the inventive compound in a suitable amounts of the solvent (organic solvent or water or a mixture thereof) at a higher than ambient temperature, and cooling the solution at a rate sufficient to form crystals which are then isolated by standard methods. Analytical techniques such as, for example, infrared spectroscopy, show the presence of the solvent (or water) in the crystals as a solvate (or hydrate).

The invention also relates to pharmaceutically acceptable prodrugs of the compounds of the invention, and treatment methods employing such pharmaceutically acceptable prodrugs. The term “prodrug” means a precursor of a designated compound that, following administration to a subject, yields the compound in vivo via a chemical or physiological process such as solvolysis or enzymatic cleavage, or under physiological conditions (e.g., a prodrug on being brought to physiological pH is converted to the compound of the invention). A “pharmaceutically acceptable prodrug” is a prodrug that is non-toxic, biologically tolerable, and otherwise suitable for formulation and/or administration to the subject. Illustrative procedures for the selection and preparation of suitable prodrug derivatives are described, for example, in “Design of Prodrugs”, ed. H. Bundgaard, Elsevier, 1985.

Examples of prodrugs include pharmaceutically acceptable esters of the compounds of the invention, which are also considered to be part of the invention. Pharmaceutically acceptable esters of the present compounds include the following groups: (1) carboxylic acid esters obtained by esterification of the hydroxy groups, in which the non-carbonyl moiety of the carboxylic acid portion of the ester grouping is selected from straight or branched chain alkyl (for example, acetyl, n-propyl, t-butyl, or n-butyl), alkoxyalkyl (for example, methoxymethyl), aralkyl (for example, benzyl), aryloxyalkyl (for example, phenoxymethyl), aryl (for example, phenyl optionally substituted with, for example, halogen, C₁₋₄alkyl, C₁₋₄alkoxy, or amino); (2) sulfonate esters, such as alkyl- or aralkylsulfonyl (for example, methanesulfonyl); (3) amino acid esters (for example, L-valyl or L-isoleucyl); (4) phosphonate esters and (5) mono-, di- or triphosphate esters. The phosphate esters may be further esterified by, for example, a C₁₋₂₀ alcohol or reactive derivative thereof, or by a 2,3-di(C6-24)acyl glycerol. Additional discussion of prodrugs is provided in T. Higuchi and V. Stella, Pro-drugs as Novel Delivery Systems (1987) 14 of the A.C.S. Symposium Series, and in Bioreversible Carriers in Drug Design, (1987) Edward B. Roche, ed., American Pharmaceutical Association and Pergamon Press.

For example, if a compound of the invention contains a carboxylic acid functional group, a prodrug can comprise an ester formed by the replacement of the hydrogen atom of the acid group with a group such as, for example, (C₁-C₈)alkyl, (C₂-C₁₂)alkanoyloxymethyl, 1-(alkanoyloxy)ethyl having from 4 to 9 carbon atoms, 1-methyl-1-(alkanoyloxy)-ethyl having from 5 to 10 carbon atoms, alkoxycarbonyloxymethyl having from 3 to 6 carbon atoms, 1-(alkoxycarbonyloxy)ethyl having from 4 to 7 carbon atoms, 1-methyl-1-(alkoxycarbonyloxy)ethyl having from 5 to 8 carbon atoms, N-(alkoxycarbonyl)aminomethyl having from 3 to 9 carbon atoms, 1-(N-(alkoxycarbonyl)amino)ethyl having from 4 to 10 carbon atoms, 3-phthalidyl, 4-crotonolactonyl, gamma-butyrolacton-4-yl, di-N,N—(C₁-C₂)alkylamino(C₂-C₃)alkyl (such as β-dimethylaminoethyl), carbamoyl-(C₁-C₂)alkyl, N,N-di(C₁-C₂)alkylcarbamoyl-(C₁-C₂)alkyl and piperidino-, pyrrolidino- or morpholine (C₂-C₃)alkyl, and the like.

Similarly, if a compound of the invention contains an alcohol functional group, a prodrug can be formed by the replacement of the hydrogen atom of the alcohol group with a group such as, for example, (C₁-C₆)alkanoyloxymethyl, 1-((C₁-C₆)alkanoyloxy)ethyl, 1-methyl-1-((C₁-C₆)alkanoyloxy)ethyl, (C₁-C₆)alkoxycarbonyloxymethyl, N—(C₁-C₆)alkoxycarbonylaminomethyl, succinoyl, (C₁-C₆)alkanoyl, α-amino(C₁-C₄)alkanyl, arylacyl and α-aminoacyl, or α-aminoacyl-α-aminoacyl, where each α-aminoacyl group is independently selected from the naturally occurring L-amino acids, P(O)(OH)₂, —P(O)(O(C₁-C₆)alkyl)₂ or glycosyl (the radical resulting from the removal of a hydroxyl group of the hemiacetal form of a carbohydrate), and the like.

If a compound of the invention incorporates an amine functional group, a prodrug can be formed by the replacement of a hydrogen atom in the amine group with a group such as, for example, R″-carbonyl, R″O-carbonyl, NR″R′-carbonyl where R″ and R′ are each independently (C₁-C₁₀)alkyl, (C₃-C₇) cycloalkyl, benzyl, or R″-carbonyl is a natural α-aminoacyl or natural α-aminoacyl, —C(OH)C(O)OY¹ wherein Y¹ is H, (C₁-C₆)alkyl or benzyl, —C(OY²)Y³ wherein Y² is (C₁-C₄) alkyl and Y³ is (C₁-C₆)alkyl, carboxy(C₁-C₆)alkyl, amino(C₁-C₄)alkyl or mono-N— or di-N,N—(C₁-C₆)alkylaminoalkyl, —C(Y⁴)Y⁵ wherein Y⁴ is H or methyl and Y⁵ is mono-N— or di-N,N—(C₁-C₆)alkylamino morpholino, piperidin-1-yl or pyrrolidin-1-yl, and the like.

The present invention also relates to pharmaceutically active metabolites of compounds of the invention, and uses of such metabolites in the methods of the invention. A “pharmaceutically active metabolite” means a pharmacologically active product of metabolism in the body of a compound of the invention or salt thereof. Prodrugs and active metabolites of a compound may be determined using routine techniques known or available in the art. See, e.g., Bertolini et al., J. Med. Chem. 1997, 40, 2011-2016; Shan et al., J. Pharm. Sci. 1997, 86 (7), 765-767; Bagshawe, Drug Dev. Res. 1995, 34, 220-230; Bodor, Adv. Drug Res. 1984, 13, 255-331; Bundgaard, Design of Prodrugs (Elsevier Press, 1985); and Larsen, Design and Application of Prodrugs, Drug Design and Development (Krogsgaard-Larsen et al., eds., Harwood Academic Publishers, 1991).

Any formula given herein is also intended to represent unlabeled forms as well as isotopically labeled forms of the compounds. Isotopically labeled compounds have structures depicted by the formulas given herein except that one or more atoms are replaced by an atom having a selected atomic mass or mass number. Examples of isotopes that can be incorporated into compounds of the invention include isotopes of hydrogen, carbon, nitrogen, oxygen, phosphorous, fluorine, chlorine, and iodine, such as ²H, ³H, ¹¹C, ¹³C, ¹⁴C, ¹⁵N, ¹⁸O, ¹⁷O, ³¹P, ³²P, ³⁵S, ¹⁸F, ³⁶Cl, and ¹²⁵I, respectively. Such isotopically labelled compounds are useful in metabolic studies (for example with ¹⁴C), reaction kinetic studies (with, for example ²H or ³H), detection or imaging techniques [such as positron emission tomography (PET) or single-photon emission computed tomography (SPECT)] including drug or substrate tissue distribution assays, or in radioactive treatment of patients. In particular, an ¹⁸F or ¹¹C labeled compound may be particularly suitable for PET or SPECT studies. Further, substitution with heavier isotopes such as deuterium (i.e., ²H) may afford certain therapeutic advantages resulting from greater metabolic stability, for example increased in vivo half-life or reduced dosage requirements. Isotopically labeled compounds of this invention and prodrugs thereof can generally be prepared by carrying out the procedures disclosed in the schemes or in the examples and preparations described below by substituting a readily available isotopically labeled reagent for a non-isotopically labeled reagent.

The use of the terms “salt,” “solvate,” “polymorph,” “prodrug,” and the like, with respect to the compounds described herein is intended to apply equally to the salt, solvate, polymorph, and prodrug forms of enantiomers, stereoisomers, rotamers, tautomers, atropisomers, and racemates of the inventive compounds.

In other embodiments the invention is a method for treating a viral disease in a subject comprising administering to said subject a therapeutically effective dose of one or more of the compounds selected from the group consisting of: 5-[(3-cyanophenyl)methyl]-2-fluoro-7-hexyl-5H,6H,7H,8H,9H,10H-cyclohepta[b]indole-4-carboxylic acid, 5-[(6-cyanopyridin-2-yl)methyl]-7-hexyl-5H,6H,7H,8H,9H,10H-cyclohepta[b]indole-4-carboxylic acid, 5-[(6-carbamoylpyridin-2-yl)methyl]-7-hexyl-5H,6H,7H,8H,9H,10H-cyclohepta[b]indole-4-carboxylic acid, 6-({4-carboxy-7-hexyl-5H,6H,7H,8H,9H,10H-cyclohepta[b]indol-5-yl}methyl)pyridine-2-carboxylic acid, 5-[(3-cyano-2-fluorophenyl)methyl]-7-hexyl-5H,6H,7H,8H,9H,10H-cyclohepta[b]indole-4-carboxylic acid, 5-[(1,3-benzoxazol-6-yl)methyl]-7-hexyl-5H,6H,7H,8H,9H,10H-cyclohepta[b]indole-4-carboxylic acid, 5-[(1,3-benzoxazol-5-yl)methyl]-7-hexyl-5H,6H,7H,8H,9H,10H-cyclohepta[b]indole-4-carboxylic acid, 5-[(6-fluoropyridin-2-yl)methyl]-7-hexyl-5H,6H,7H,8H,9H,10H-cyclohepta[b]indole-4-carboxylic acid, 5-[(2-fluoropyridin-4-yl)methyl]-7-hexyl-5H,6H,7H,8H,9H,10H-cyclohepta[b]indole-4-carboxylic acid, 7-hexyl-5-{[6-(trifluoromethyl)pyridin-2-yl]methyl}-5H,6H,7H,8H,9H,10H-cyclohepta[b]indole-4-carboxylic acid, 7-hexyl-5-{[2-(trifluoromethyl)pyridin-4-yl]methyl}-5H,6H,7H,8H,9H,10H-cyclohepta[b]indole-4-carboxylic acid, 5-[(5-cyanopyridin-3-yl)methyl]-7-hexyl-5H,6H,7H,8H,9H,10H-cyclohepta[b]indole-4-carboxylic acid, 5-[(5-cyanothiophen-2-yl)methyl]-7-hexyl-5H,6H,7H,8H,9H,10H-cyclohepta[b]indole-4-carboxylic acid, 5-[(4-cyanothiophen-2-yl)methyl]-7-hexyl-5H,6H,7H,8H,9H,10H-cyclohepta[b]indole-4-carboxylic acid, 5-[(5-cyanofuran-2-yl)methyl]-7-hexyl-5H,6H,7H,8H,9H,10H-cyclohepta[b]indole-4-carboxylic acid, 5-[(3,5-dimethyl-1,2-oxazol-4-yl)methyl]-7-hexyl-5H,6H,7H,8H,9H,10H-cyclohepta[b]indole-4-carboxylic acid, 5-(3-cyanobenzoyl)-7-hexyl-5H,6H,7H,8H,9H,10H-cyclohepta[b]indole-4-carboxylic acid, 5-[(1,3-benzoxazol-7-yl)methyl]-7-hexyl-5H,6H,7H,8H,9H,10H-cyclohepta[b]indole-4-carboxylic acid, 5-[(5-cyanothiophen-3-yl)methyl]-7-hexyl-5H,6H,7H,8H,9H,10H-cyclohepta[b]indole-4-carboxylic acid, 7-hexyl-5-[(1H-indol-4-yl)methyl]-5H,6H,7H,8H,9H,10H-cyclohepta[b]indole-4-carboxylic acid, 5-[(3-carbamoylphenyl)methyl]-7-propyl-5H,6H,7H,8H,9H,10H-cyclohepta[b]indole-4-carboxylic acid, 7-butyl-5-[(3-carbamoylphenyl)methyl]-5H,6H,7H,8H,9H,10H-cyclohepta[b]indole-4-carboxylic acid, 7-butyl-5-[(3-cyanophenyl)methyl]-5H,6H,7H,8H,9H,10H-cyclohepta[b]indole-4-carboxylic acid, 7-butyl-5-[(pyridin-3-yl)methyl]-5H,6H,7H,8H,9H,10H-cyclohepta[b]indole-4-carboxylic acid, 7-butyl-5-[(3-methylphenyl)methyl]-5H,6H,7H,8H,9H,10H-cyclohepta[b]indole-4-carboxylic acid, 7-butyl-5-[(3-methoxyphenyl)methyl]-5H,6H,7H,8H,9H,10H-cyclohepta[b]indole-4-carboxylic acid, 7-butyl-5-[(3-chlorophenyl)methyl]-5H,6H,7H,8H,9H,10H-cyclohepta[b]indole-4-carboxylic acid, 7-butyl-5-[(3-hydroxyphenyl)methyl]-5H,6H,7H,8H,9H,10H-cyclohepta[b]indole-4-carboxylic acid, 7-butyl-5-[(2-methoxypyridin-4-yl)methyl]-5H,6H,7H,8H,9H,10H-cyclohepta[b]indole-4-carboxylic acid, 7-butyl-5-[(4-carbamoylphenyl)methyl]-5H,6H,7H,8H,9H,10H-cyclohepta[b]indole-4-carboxylic acid, 7-butyl-5-[(2-carbamoylphenyl)methyl]-5H,6H,7H,8H,9H,10H-cyclohepta[b]indole-4-carboxylic acid, 7-butyl-5-[(4-methylphenyl)methyl]-5H,6H,7H,8H,9H,10H-cyclohepta[b]indole-4-carboxylic acid, 7-butyl-5-[(2-cyanophenyl)methyl]-5H,6H,7H,8H,9H,10H-cyclohepta[b]indole-4-carboxylic acid, 7-butyl-5-[(2-methylphenyl)methyl]-5H,6H,7H,8H,9H,10H-cyclohepta[b]indole-4-carboxylic acid, 7-butyl-5-[(2-fluorophenyl)methyl]-5H,6H,7H,8H,9H,10H-cyclohepta[b]indole-4-carboxylic acid, 5-[(3-carbamoylphenyl)methyl]-7-pentyl-5H,6H,7H,8H,9H,10H-cyclohepta[b]indole-4-carboxylic acid, 5-[(3-cyanophenyl)methyl]-7-pentyl-5H,6H,7H,8H,9H,10H-cyclohepta[b]indole-4-carboxylic acid, 5-[(3-carbamoylphenyl)methyl]-7-(2-phenylethyl)-5H,6H,7H,8H,9H,10H-cyclohepta[b]indole-4-carboxylic acid, 5-[(3-cyanophenyl)methyl]-7-(2-phenylethyl)-5H,6H,7H,8H,9H,10H-cyclohepta[b]indole-4-carboxylic acid, 5-[(3-carbamoylphenyl)methyl]-7-hexyl-5H,6H,7H,8H,9H,10H-cyclohepta[b]indole-4-carboxylic acid, 5-[(3-cyanophenyl)methyl]-7-hexyl-5H,6H,7H,8H,9H,10H-cyclohepta[b]indole-4-carboxylic acid, 5-[(3-carbamoylphenyl)methyl]-7-octyl-5H,6H,7H,8H,9H,10H-cyclohepta[b]indole-4-carboxylic acid, 5-[(3-cyanophenyl)methyl]-7-octyl-5H,6H,7H,8H,9H,10H-cyclohepta[b]indole-4-carboxylic acid, 5-[(3-fluorophenyl)methyl]-7-hexyl-5H,6H,7H,8H,9H,10H-cyclohepta[b]indole-4-carboxylic acid, 7-hexyl-5-[(pyridin-3-yl)methyl]-5H,6H,7H,8H,9H,10H-cyclohepta[b]indole-4-carboxylic acid, 7-hexyl-5-[(3-methylphenyl)methyl]-5H,6H,7H,8H,9H,10H-cyclohepta[b]indole-4-carboxylic acid, 7-hexyl-5-[(3-methoxyphenyl)methyl]-5H,6H,7H,8H,9H,10H-cyclohepta[b]indole-4-carboxylic acid, 5-[(3-chlorophenyl)methyl]-7-hexyl-5H,6H,7H,8H,9H,10H-cyclohepta[b]indole-4-carboxylic acid, 7-hexyl-5-[(2-methoxypyridin-4-yl)methyl]-5H,6H,7H,8H,9H,10H-cyclohepta[b]indole-4-carboxylic acid, 5-[(3-carboxyphenyl)methyl]-7-hexyl-5H,6H,7H,8H,9H,10H-cyclohepta[b]indole-4-carboxylic acid, 5-[(4-carbamoylphenyl)methyl]-7-hexyl-5H,6H,7H,8H,9H,10H-cyclohepta[b]indole-4-carboxylic acid, 5-[(2-carbamoylphenyl)methyl]-7-hexyl-5H,6H,7H,8H,9H,10H-cyclohepta[b]indole-4-carboxylic acid, 7-hexyl-5-[(4-methylphenyl)methyl]-5H,6H,7H,8H,9H,10H-cyclohepta[b]indole-4-carboxylic acid, 5-[(4-cyanophenyl)methyl]-7-hexyl-5H,6H,7H,8H,9H,10H-cyclohepta[b]indole-4-carboxylic acid, 5-[(2-cyanophenyl)methyl]-7-hexyl-5H,6H,7H,8H,9H,10H-cyclohepta[b]indole-4-carboxylic acid, 7-hexyl-5-[(2-methylphenyl)methyl]-5H,6H,7H,8H,9H,10H-cyclohepta[b]indole-4-carboxylic acid, 5-[(2-fluorophenyl)methyl]-7-hexyl-5H,6H,7H,8H,9H,10H-cyclohepta[b]indole-4-carboxylic acid, 5-[(4-fluorophenyl)methyl]-7-hexyl-5H,6H,7H,8H,9H,10H-cyclohepta[b]indole-4-carboxylic acid, 9-[(3-carbamoylphenyl)methyl]-2-hexyl-2,3,4,9-tetrahydro-1H-carbazole-8-carboxylic acid, 9-[(3-cyanophenyl)methyl]-2-hexyl-2,3,4,9-tetrahydro-1H-carbazole-8-carboxylic acid, 9-[(3-carboxyphenyl)methyl]-2-hexyl-2,3,4,9-tetrahydro-1H-carbazole-8-carboxylic acid, 9-[(3-fluorophenyl)methyl]-2-hexyl-2,3,4,9-tetrahydro-1H-carbazole-8-carboxylic acid, 2-hexyl-9-[(pyridin-3-yl)methyl]-2,3,4,9-tetrahydro-1H-carbazole-8-carboxylic acid, 2-hexyl-9-[(3-methylphenyl)methyl]-2,3,4,9-tetrahydro-1H-carbazole-8-carboxylic acid, 2-hexyl-9-[(3-methoxyphenyl)methyl]-2,3,4,9-tetrahydro-1H-carbazole-8-carboxylic acid, 9-[(3-chlorophenyl)methyl]-2-hexyl-2,3,4,9-tetrahydro-1H-carbazole-8-carboxylic acid, 2-hexyl-9-[(3-hydroxyphenyl)methyl]-2,3,4,9-tetrahydro-1H-carbazole-8-carboxylic acid, 2-hexyl-9-[(2-methoxypyridin-4-yl)methyl]-2,3,4,9-tetrahydro-1H-carbazole-8-carboxylic acid, 9-[(3-carboxyphenyl)methyl]-2-hexyl-2,3,4,9-tetrahydro-1H-carbazole-8-carboxylic acid, 9-[(4-carbamoylphenyl)methyl]-2-hexyl-2,3,4,9-tetrahydro-1H-carbazole-8-carboxylic acid, 9-[(2-carbamoylphenyl)methyl]-2-hexyl-2,3,4,9-tetrahydro-1H-carbazole-8-carboxylic acid, 2-hexyl-9-[(4-methylphenyl)methyl]-2,3,4,9-tetrahydro-1H-carbazole-8-carboxylic acid, 9-[(4-cyanophenyl)methyl]-2-hexyl-2,3,4,9-tetrahydro-1H-carbazole-8-carboxylic acid, 9-[(2-cyanophenyl)methyl]-2-hexyl-2,3,4,9-tetrahydro-1H-carbazole-8-carboxylic acid, 2-hexyl-9-[(2-methylphenyl)methyl]-2,3,4,9-tetrahydro-1H-carbazole-8-carboxylic acid, 9-[(2-fluorophenyl)methyl]-2-hexyl-2,3,4,9-tetrahydro-1H-carbazole-8-carboxylic acid, 9-[(3-carbamoylphenyl)methyl]-2-(2-phenylethyl)-2,3,4,9-tetrahydro-1H-carbazole-8-carboxylic acid, 9-[(3-cyanophenyl)methyl]-2-(2-phenylethyl)-2,3,4,9-tetrahydro-1H-carbazole-8-carboxylic acid, 9-[(3-carbamoylphenyl)methyl]-4-(2-phenylethyl)-2,3,4,9-tetrahydro-1H-carbazole-8-carboxylic acid, 9-[(3-cyanophenyl)methyl]-4-(2-phenylethyl)-2,3,4,9-tetrahydro-1H-carbazole-8-carboxylic acid, 9-[(3-carbamoylphenyl)methyl]-2-propyl-2,3,4,9-tetrahydro-1H-carbazole-8-carboxylic acid, 9-[(3-cyanophenyl)methyl]-2-propyl-2,3,4,9-tetrahydro-1H-carbazole-8-carboxylic acid, 2-butyl-9-[(3-carbamoylphenyl)methyl]-2,3,4,9-tetrahydro-1H-carbazole-8-carboxylic acid, 9-[(3-carbamoylphenyl)methyl]-2-pentyl-2,3,4,9-tetrahydro-1H-carbazole-8-carboxylic acid, 9-[(3-cyanophenyl)methyl]-2-pentyl-2,3,4,9-tetrahydro-1H-carbazole-8-carboxylic acid, 1-butyl-9-[(3-carbamoylphenyl)methyl]-2,3,4,9-tetrahydro-1H-carbazole-8-carboxylic acid, 9-[(3-carbamoylphenyl)methyl]-1-(pentyloxy)-2,3,4,9-tetrahydro-1H-carbazole-8-carboxylic acid, 9-[(3-cyanophenyl)methyl]-1-(pentyloxy)-2,3,4,9-tetrahydro-1H-carbazole-8-carboxylic acid, 1-butyl-9-[(3-cyanophenyl)methyl]-2,3,4,9-tetrahydro-1H-carbazole-8-carboxylic acid, 6-butyl-5-[(3-carbamoylphenyl)methyl]-5H,6H,7H,8H,9H,10H-cyclohepta[b]indole-4-carboxylic acid, 6-butyl-5-[(3-cyanophenyl)methyl]-5H,6H,7H,8H,9H,10H-cyclohepta[b]indole-4-carboxylic acid, 9-[(3-carbamoylphenyl)methyl]-1-ethyl-2,3,4,9-tetrahydro-1H-carbazole-8-carboxylic acid, 9-[(3-cyanophenyl)methyl]-1-ethyl-2,3,4,9-tetrahydro-1H-carbazole-8-carboxylic acid, 9-[(3-carboxyphenyl)methyl]-1-ethyl-2,3,4,9-tetrahydro-1H-carbazole-8-carboxylic acid, 9-[(3-carbamoylphenyl)methyl]-1-propoxy-2,3,4,9-tetrahydro-1H-carbazole-8-carboxylic acid, 9-[(3-carbamoylphenyl)methyl]-4-ethyl-2,3,4,9-tetrahydro-1H-carbazole-8-carboxylic acid, 4-[(3-carbamoylphenyl)methyl]-3-ethyl-1H,2H,3H,4H-cyclopenta[b]indole-5-carboxylic acid, 4-[(3-cyanophenyl)methyl]-3-ethyl-1H,2H,3H,4H-cyclopenta[b]indole-5-carboxylic acid, 3-butyl-4-[(3-carbamoylphenyl)methyl]-1H,2H,3H,4H-cyclopenta[b]indole-5-carboxylic acid, 3-butyl-4-[(3-cyanophenyl)methyl]-1H,2H,3H,4H-cyclopenta[b]indole-5-carboxylic acid, 2-butyl-4-[(3-carbamoylphenyl)methyl]-1H,2H,3H,4H-cyclopenta[b]indole-5-carboxylic acid, 2-butyl-4-[(3-cyanophenyl)methyl]-1H,2H,3H,4H-cyclopenta[b]indole-5-carboxylic acid, 5-[(3-carbamoylphenyl)methyl]-10-ethyl-5H,6H,7H,8H,9H,10H-cyclohepta[b]indole-4-carboxylic acid, 5-[(3-cyanophenyl)methyl]-10-ethyl-5H,6H,7H,8H,9H,10H-cyclohepta[b]indole-4-carboxylic acid, 5-[(3-carbamoylphenyl)methyl]-10-propyl-5H,6H,7H,8H,9H,10H-cyclohepta[b]indole-4-carboxylic acid, 5-[(3-cyanophenyl)methyl]-10-propyl-5H,6H,7H,8H,9H,10H-cyclohepta[b]indole-4-carboxylic acid, 9-[(3-carboxyphenyl)methyl]-4-ethyl-2,3,4,9-tetrahydro-1H-carbazole-8-carboxylic acid, 9-[(3-carbamoylphenyl)methyl]-3-ethyl-2,3,4,9-tetrahydro-1H-carbazole-8-carboxylic acid, 10-butyl-5-[(3-carbamoylphenyl)methyl]-5H,6H,7H,8H,9H,10H-cyclohepta[b]indole-4-carboxylic acid, 10-butyl-5-[(3-cyanophenyl)methyl]-5H,6H,7H,8H,9H,10H-cyclohepta[b]indole-4-carboxylic acid, 5-[(3-carbamoylphenyl)methyl]-10-pentyl-5H,6H,7H,8H,9H,10H-cyclohepta[b]indole-4-carboxylic acid, 5-[(3-cyanophenyl)methyl]-10-pentyl-5H,6H,7H,8H,9H,10H-cyclohepta[b]indole-4-carboxylic acid, 4-[(3-carbamoylphenyl)methyl]-2-pentyl-1H,2H,3H,4H-cyclopenta[b]indole-5-carboxylic acid, 4-[(3-cyanophenyl)methyl]-2-pentyl-1H,2H,3H,4H-cyclopenta[b]indole-5-carboxylic acid, 9-[(3-carbamoylphenyl)methyl]-2-ethyl-2,3,4,9-tetrahydro-1H-carbazole-8-carboxylic acid, 9-[(3-carboxyphenyl)methyl]-2-ethyl-2,3,4,9-tetrahydro-1H-carbazole-8-carboxylic acid, 5-[(3-carbamoylphenyl)methyl]-7-ethyl-5H,6H,7H,8H,9H,10H-cyclohepta[b]indole-4-carboxylic acid, 5-[(3-cyanophenyl)methyl]-7-ethyl-5H,6H,7H,8H,9H,10H-cyclohepta[b]indole-4-carboxylic acid, 9-[(3-cyanophenyl)methyl]-3-ethyl-2,3,4,9-tetrahydro-1H-carbazole-8-carboxylic acid, 9-[(3-carbamoylphenyl)methyl]-4-propyl-2,3,4,9-tetrahydro-1H-carbazole-8-carboxylic acid, 9-[(3-carbamoylphenyl)methyl]-3-propyl-2,3,4,9-tetrahydro-1H-carbazole-8-carboxylic acid, 9-[(3-cyanophenyl)methyl]-3-propyl-2,3,4,9-tetrahydro-1H-carbazole-8-carboxylic acid, 4-butyl-9-[(3-carbamoylphenyl)methyl]-2,3,4,9-tetrahydro-1H-carbazole-8-carboxylic acid, 4-butyl-9-[(3-cyanophenyl)methyl]-2,3,4,9-tetrahydro-1H-carbazole-8-carboxylic acid, 3-butyl-9-[(3-carbamoylphenyl)methyl]-2,3,4,9-tetrahydro-1H-carbazole-8-carboxylic acid, 3-butyl-9-[(3-cyanophenyl)methyl]-2,3,4,9-tetrahydro-1H-carbazole-8-carboxylic acid, 9-[(3-carbamoylphenyl)methyl]-4-pentyl-2,3,4,9-tetrahydro-1H-carbazole-8-carboxylic acid, 9-[(3-cyanophenyl)methyl]-4-pentyl-2,3,4,9-tetrahydro-1H-carbazole-8-carboxylic acid, 9-[(3-carbamoylphenyl)methyl]-3-pentyl-2,3,4,9-tetrahydro-1H-carbazole-8-carboxylic acid, 9-[(3-cyanophenyl)methyl]-3-pentyl-2,3,4,9-tetrahydro-1H-carbazole-8-carboxylic acid, 4-[(3-carbamoylphenyl)methyl]-3-propyl-1H,2H,3H,4H-cyclopenta[b]indole-5-carboxylic acid, 4-[(3-cyanophenyl)methyl]-3-propyl-1H,2H,3H,4H-cyclopenta[b]indole-5-carboxylic acid, 2-({7-butyl-5-[(3-carbamoylphenyl)methyl]-5H,6H,7H,8H,9H,10H-cyclohepta[b]indol-4-yl}formamido)acetic acid, 2-({7-butyl-5-[(3-cyanophenyl)methyl]-5H,6H,7H,8H,9H,10H-cyclohepta[b]indol-4-yl}formamido)acetic acid, 7-butyl-5-[(3-carbamoylphenyl)methyl]-N-(2-hydroxyethyl)-5H,6H,7H,8H,9H,10H-cyclohepta[b]indole-4-carboxamide, 7-butyl-5-[(3-cyanophenyl)methyl]-N-(2-hydroxyethyl)-5H,6H,7H,8H,9H,10H-cyclohepta[b]indole-4-carboxamide, 7-butyl-5-[(3-fluorophenyl)methyl]-5H,6H,7H,8H,9H,10H-cyclohepta[b]indole-4-carboxylic acid, and 7-butyl-5-[(3-carboxyphenyl)methyl]-5H,6H,7H,8H,9H,10H-cyclohepta[b]indole-4-carboxylic acid, or pharmaceutically acceptable salts or stereoisomers thereof.

Yet in other embodiments the invention is a method for treating a viral disease in a subject comprising administering to said subject a therapeutically effective dose of a compound selected from the group consisting of: 2-((2′-(5-Ethyl-3,4-diphenyl-1H-pyrazol-1-yl)-[1,1′-biphenyl]-3-yl)oxy)acetic acid, 6-chloro-4-phenyl-2-(piperidin-1-yl)-3-(1H-tetrazol-5-yl)quinoline, 5-((3-chloro-2-methylphenoxy)methyl)-2-phenyl-[1,2,4]triazolo[1,5-a]pyrimidin-7(4H)-one, and 3-(5-cyclopropyl-3-(3,5-dimethyl-1H-pyrazol-4-yl)-2-(3-isopropoxyphenyl)-1H-indol-1-yl)propanoic acid, or pharmaceutically acceptable salts or stereoisomers thereof. It should be appreciated that WO 00/59506, U.S. 2013/0116234 A1, WO 2010/056631, PCT/US21/39470, U.S. 2013/0116231 A1, Sulsky et al., Bioorg Med Chem Lett. 2007 Jun. 15; 17(12):3511-5. doi: 10.1016/j.bmc.2006.12.044. Epub 2006 Dec. 21, Kuhne et al., Bioorg Med Chem Lett. 2016 Oct. 15; 26(20):5092-5097. doi: 10.1016/j.bmcl.2016.08.071. Epub 2016 Aug. 22.), and Lan et al., J Lipid Res. 2011 April; 52(4):646-56. doi: 10.1194/jlr.M012757. Epub 2011 Feb. 4.), Tagami et al., ACS Med Chem Lett. 2016 Apr. 14; 7(4): 435-439. doi: 10.1021/acsmedchemlett.6b00040) are hereby incorporated by reference in their entirety.

It is an object of the present invention to utilize the compounds described herein in the treatment of viral disorders by acting on the fatty acid binding protein (FABP4).

Yet another object of the present invention is a pharmaceutical composition comprising a compounds herein as active ingredient, in combination with a pharmaceutically acceptable diluent or carrier for use in the treatment of viral disorders by acting on FABP4. Here, the pharmaceutical composition can further comprise an additional therapeutically active agent.

Yet another object of the present invention is a method for the treatment of viral disorders by acting on the FABP4, which comprises administering to a subject in need of such treatment (preferably, a human) an effective amount of the compounds herein, including, optionally, the co-administration with other therapeutic agents, either as a single (or multiple) dosing, and either simultaneously or sequentially.

Yet another embodiment is a method for administering a compound of the instant invention to a subject (e.g., a human) in need thereof by administering to the subject the pharmaceutical formulation of the present invention.

Yet another embodiment is a method of preparing a pharmaceutical formulation of the present invention by mixing at least one pharmaceutically acceptable compound of the present invention, and, optionally, one or more pharmaceutically acceptable additives or excipients.

For preparing pharmaceutical compositions from the compounds described by this invention, inert, pharmaceutically acceptable carriers can be either solid or liquid. Solid form preparations include powders, tablets, dispersible granules, capsules, cachets and suppositories. The powders and tablets may be comprised of from about 5 to about 95 percent active ingredient. Suitable solid carriers are known in the art, e.g., magnesium carbonate, magnesium stearate, talc, sugar or lactose. Tablets, powders, cachets and capsules can be used as solid dosage forms suitable for oral administration. Examples of pharmaceutically acceptable carriers and methods of manufacture for various compositions may be found in A. Gennaro (ed.), Remington's Pharmaceutical Sciences, 18th Edition, (1990), Mack Publishing Co., Easton, Pa.

The compositions and formulations of the invention can be administered as sterile compositions and sterile formulations. Sterile pharmaceutical formulations are compounded or manufactured according to pharmaceutical-grade sterilization standards (e.g., United States Pharmacopeia Chapters 797, 1072, and 1211; California Business & Professions Code 4127.7; 16 California Code of Regulations 1751, 21 Code of Federal Regulations 21, or ex-U.S. counterparts to such regulations) known to those of skill in the art.

Liquid form preparations include solutions, suspensions and emulsions. As an example, may be mentioned water or water-propylene glycol solutions for parenteral injection or addition of sweeteners and opacifiers for oral solutions, suspensions, and emulsions. Liquid form preparations may also include solutions for intranasal administration.

Aerosol preparations suitable for inhalation may include solutions and solids in powder form, which may be in combination with a pharmaceutically acceptable carrier, such as an inert compressed gas, e.g. nitrogen.

Methods of delivering drugs by pulmonary administration have been described. For example, each of U.S. Pat. Nos. 6,550,472, 6,546,927, 6,543,443, 6,540,154, 6,540,153, 6,467,476, 6,427,682, 6,503,480, 6,447,753, 6,387,390, 5,985,320, 5,985,309, 5,855,913, 6,431,167, 6,408,854, 6,349,719, 6,167,880, 6,098,620, 5,971,951, 5,957,124, 5,906,202, 5,819,726, 5,755,218, 5,522,385, 6,546,929, 6,543,448, 6,509,006, 6,423,344, 6,303,582, and 6,138,668 teaches methods and devices useful in the pulmonary administration of drugs and/or nasal instillation. Bioadhesives have been described for facilitating transport of medicaments across endothelial mucosa. For example, U.S. Pat. No. 6,228,383 teaches use of bioadhesive fatty acid esters for facilitating transport of drug substances across mucosa in the lung, nose and other tissues. Penetration enhancers have been described in, for example, U.S. patent application Ser. No. 09/315,298, filed on May 20, 1999. Penetration enhancers facilitate the penetration of mucosa, including pulmonary and nasal mucosa. The present invention provides, inter alia, compositions formulated for pulmonary or nasal administration of antiviral compounds, especially compounds capable of attenuating, mitigating or preventing viral infections, and especially coronavirus. In some embodiments of the invention, viral infections are treated by administering an antiviral compound of the present invention to a patient in need thereof. In some embodiments of the invention, viral infection is treated by administering an antiviral small molecule to a patient in need thereof. In some embodiments according to the present invention, an antiviral agent is administered by pulmonary or intranasal means to a patient in need thereof. In certain embodiments, the antiviral agent is a compound of the invention. In other embodiments of the invention, the antiviral agent is a mixture of antiviral compounds. In certain embodiments, the invention comprises a molecule of the invention in combination therapy, for example with one or more additional antiviral agents. In some embodiments of the present invention, the inventive composition comprises, in addition to one or more antiviral agents, a therapeutically acceptable agent for intrapulmonary or intranasal administration.

Also included are solid form preparations that are intended to be converted, shortly before use, to liquid form preparations for either oral or parenteral administration. Such liquid forms include solutions, suspensions and emulsions.

The compounds of the invention may also be deliverable transdermally. The transdermal compositions can take the form of creams, lotions, aerosols and/or emulsions and can be included in a transdermal patch of the matrix or reservoir type as are conventional in the art for this purpose.

The compounds of this invention may also be delivered subcutaneously.

The compound can be administered orally or intravenously.

The pharmaceutical preparation can be in a unit dosage form. In such form, the preparation is subdivided into suitably sized unit doses containing appropriate quantities of the active component, e.g., an effective amount to achieve the desired purpose.

The quantity of active compound in a unit dose of preparation may be varied or adjusted from about 1 mg to about 1000 mg, for example from about 1 mg to about 500 mg, in particular from about 1 mg to about 250 mg, or from about 1 mg to about 25 mg, according to the particular application.

The actual dosage employed may be varied depending upon the requirements of the patient and the severity of the condition being treated. Determination of the proper dosage regimen for a particular situation is within the skill of the art. For convenience, the total daily dosage may be divided and administered in portions during the day as required.

The amount and frequency of administration of the compounds of the invention and/or the pharmaceutically acceptable salts thereof will be regulated according to the judgment of the attending clinician considering such factors as age, condition and size of the patient as well as severity of the symptoms being treated. A typical recommended daily dosage regimen for oral administration can range from about 1 mg/day to about 500 mg/day, preferably 1 mg/day to 200 mg/day, in two to four divided doses.

Treatment or Prevention of Viral Infection

The compounds of the invention are useful in human and veterinary medicine for treating or preventing a viral infection in a patient. In one embodiment, the compounds of the invention are inhibitors of viral replication. In another embodiment, the compounds of the invention can be inhibitors of influenza, rhinovirus or coronavirus replication. Accordingly, the compounds of the invention are useful for treating viral infections, such as coronavirus. In accordance with the invention, the compounds of the invention can be administered to a patient in need of treatment or prevention of a viral infection.

Accordingly, in one embodiment, the invention provides methods for treating a viral infection in a patient comprising administering to the patient an effective amount of at least one compounds of the invention or a pharmaceutically acceptable salt thereof.

The compounds of the invention are useful in the inhibition of viruses, the treatment of viral infection and/or reduction of the likelihood or severity of symptoms of viral infection and the inhibition of viral replication and/or viral production in a cell-based system. For example, the compounds of the invention are useful in treating infection by viruses after suspected past exposure to viruses by such means as airborne transmission, blood transfusion, exchange of body fluids, etc.

In one embodiment, the viral infection is acute respiratory viral infection. In another embodiment, the viral infection is chronic viral infection.

Accordingly, in one embodiment, the invention provides methods for treating viral infection in a patient, the methods comprising administering to the patient an effective amount of at least one the invention or a pharmaceutically acceptable salt thereof. In a specific embodiment, the amount administered is effective to treat or prevent a viral infection in a patient. In another specific embodiment, the amount administered is effective to inhibit viral replication and/or viral production in the patient.

In particular embodiments, the viral infection is caused by influenza, coronavirus, rhinovirus, respiratory syncytial viruses (RSVs), parainfluenza, adenoviruses, Human metapneumovirus (HMPV) or Human Bocavirus (HBoV).

Combination Therapy

In one embodiment, one or more compounds of the present invention are administered with one or more additional therapeutic agents selected from: an interferon, an immunomodulator, a viral replication inhibitor, an antisense agent, a therapeutic vaccine, a viral polymerase inhibitor, a nucleoside inhibitor, a viral protease inhibitor, a viral helicase inhibitor, a virion production inhibitor, a viral entry inhibitor, a viral assembly inhibitor, an antibody therapy (monoclonal or polyclonal), and any agent useful for treating an RNA-dependent polymerase-related disorder.

In another embodiment, the present methods for treating or preventing viral infection can further comprise the administration of one or more additional therapeutic agents.

In one embodiment, the additional therapeutic agent is an antiviral agent.

In another embodiment, the additional therapeutic agent is an immunomodulatory agent, such as an immunosuppressive agent.

Accordingly, in one embodiment, the present invention provides methods for treating a viral infection in a patient, the method comprising administering to the patient: a compound of the invention or a pharmaceutically acceptable salt thereof, and (ii) at least one additional therapeutic agent that is other than a compound of the invention, wherein the amounts administered are together effective to treat or prevent a viral infection.

When administering a combination therapy of the invention to a patient, therapeutic agents in the combination, or a pharmaceutical composition or compositions comprising therapeutic agents, may be administered in any order such as, for example, sequentially, concurrently, together, simultaneously and the like. The amounts of the various actives in such combination therapy may be different amounts (different dosage amounts) or same amounts (same dosage amounts). Thus, for non-limiting illustration purposes, a compound of the invention and an additional therapeutic agent may be present in fixed amounts (dosage amounts) in a single dosage unit (e.g., a capsule, a tablet and the like).

In one embodiment, the at least one a compound of the invention is administered during a time when the additional therapeutic agent(s) exert their prophylactic or therapeutic effect, or vice versa.

In another embodiment, the at least one a compound of the invention and the additional therapeutic agent(s) are administered in doses commonly employed when such agents are used as monotherapy for treating a viral infection.

In another embodiment, the at least one a compound of the invention and the additional therapeutic agent(s) are administered in doses lower than the doses commonly employed when such agents are used as monotherapy for treating a viral infection.

In still another embodiment, the at least one a compound of the invention and the additional therapeutic agent(s) act synergistically and are administered in doses lower than the doses commonly employed when such agents are used as monotherapy for treating a viral infection.

In one embodiment, the at least one a compound of the invention and the additional therapeutic agent(s) are present in the same composition. In one embodiment, this composition is suitable for oral administration. In another embodiment, this composition is suitable for intravenous administration. In another embodiment, this composition is suitable for subcutaneous administration. In still another embodiment, this composition is suitable for parenteral administration.

Viral infections and virus-related disorders that can be treated or prevented using the combination therapy methods of the present invention include, but are not limited to, those listed above.

The at least one a compound of the invention and the additional therapeutic agent(s) can act additively or synergistically. A synergistic combination may allow the use of lower dosages of one or more agents and/or less frequent administration of one or more agents of a combination therapy. A lower dosage or less frequent administration of one or more agents may lower toxicity of therapy without reducing the efficacy of therapy.

In one embodiment, the administration of at least one compound of the invention and the additional therapeutic agent(s) may inhibit the resistance of a viral infection to these agents.

Non-limiting examples of additional therapeutic agents useful in the present combination therapy compositions and methods include an interferon, an immunomodulator, a viral replication inhibitor, an antisense agent, a therapeutic vaccine, a viral polymerase inhibitor, a nucleoside inhibitor, a viral protease inhibitor, a viral helicase inhibitor, a virion production inhibitor, a viral entry inhibitor, a viral assembly inhibitor, an antibody therapy (monoclonal or polyclonal), and any agent useful for treating an RNA-dependent polymerase-related disorder.

In one embodiment, the additional therapeutic agent is a viral protease inhibitor.

In another embodiment, the additional therapeutic agent is a viral replication inhibitor.

In another embodiment, the Compounds of the invention are in substantially purified form.

Other embodiments of the present invention include the following:

(a) A pharmaceutical composition comprising an effective amount of a compound of the invention or a pharmaceutically acceptable salt thereof, and a pharmaceutically acceptable carrier.

(b) The pharmaceutical composition of (a), further comprising a second therapeutic agent selected from the group consisting of antiviral agents, immunomodulators, and anti-infective agents.

(c) The pharmaceutical composition of (b), wherein the antiviral agent is an antiviral selected from the group consisting of protease inhibitors, polymerase inhibitors and other viral inhibitors.

(d) A pharmaceutical combination that is (i) a compound of the invention and (ii) a second therapeutic agent selected from the group consisting of antiviral agents, immunomodulators, and anti-infective agents; wherein the compound of the invention and the second therapeutic agent are each employed in an amount that renders the combination effective for inhibiting viral replication or disease, or for treating viral infection and/or reducing the likelihood or severity of symptoms of viral infection.

(e) The combination of (d), wherein the antiviral agent is an antiviral selected from the group consisting of protease inhibitors, polymerase inhibitors and other viral inhibitors.

(f) A method of inhibiting viral replication in a subject in need thereof which comprises administering to the subject an effective amount of a compound of the invention.

(g) A method of treating viral infection and/or reducing the likelihood or severity of symptoms of viral infection in a subject in need thereof which comprises administering to the subject an effective amount of a compound of the invention).

(h) The method of (g), wherein the compound of the invention is administered in combination with an effective amount of at least one second therapeutic agent selected from the group consisting of antiviral agents, immunomodulators, and anti-infective agents.

(i) The method of (h), wherein the antiviral agent is an antiviral selected from the group consisting of protease inhibitors, polymerase inhibitors and other viral inhibitors.

(j) A method of inhibiting viral replication in a subject in need thereof which comprises administering to the subject the pharmaceutical composition of (a), (b) or (c) or the combination of (d) or (e).

(k) A method of treating viral infection and/or reducing the likelihood or severity of symptoms of viral infection in a subject in need thereof which comprises administering to the subject the pharmaceutical composition of (a), (b) or (c) or the combination of (d) or (e).

The present invention also includes a compound of the present invention for use (i) in, (ii) as a medicament for, or (iii) in the preparation of a medicament for: (a) medicine, (b) inhibiting viral replication or (c) treating viral infection and/or reducing the likelihood or severity of symptoms of viral infection. In these uses, the compounds of the present invention can optionally be employed in combination with one or more second therapeutic agents selected from antiviral agents, anti-infective agents, and immunomodulators.

Additional embodiments of the invention include the pharmaceutical compositions, combinations and methods set forth in (a)-(k) above and the uses set forth in the preceding paragraph, wherein the compound of the present invention employed therein is a compound of one of the embodiments, aspects, classes, sub-classes, or features of the compounds described above. In all of these embodiments, the compound may optionally be used in the form of a pharmaceutically acceptable salt or hydrate as appropriate. It is understood that references to compounds would include the compound in its present form as well as in different forms, such as polymorphs, solvates and hydrates, as applicable.

It is further to be understood that the embodiments of compositions and methods provided as (a) through (k) above are understood to include all embodiments of the compounds, including such embodiments as result from combinations of embodiments.

### SCHEMES AND EXAMPLES

Exemplary, non-limiting, chemical entities and methods useful in preparing compounds of the invention will now be described by reference to illustrative synthetic schemes for their general preparation below and the specific examples that follow. Those skilled in the art will appreciate that other synthetic routes may be used to synthesize the compounds according to the invention. Although specific starting materials and reagents are depicted and discussed herein, other starting materials and reagents can be easily substituted to provide a variety of derivatives and/or reaction conditions. In addition, many of the exemplary compounds prepared by the described methods can be further modified in light of this disclosure using conventional chemistry well known to those skilled in the art.

Artisans will recognize that, to obtain the various compounds herein, starting materials may be suitably selected so that the ultimately desired substituents will be carried through the reaction scheme with or without protection as appropriate to yield the desired product. Alternatively, it may be necessary or desirable to employ, in the place of the ultimately desired substituent, a suitable group that may be carried through the reaction scheme and replaced as appropriate with the desired substituent. Each of the reactions depicted in the reaction schemes is preferably run at a temperature from about 0° C. to the reflux temperature of the solvent used. Unless otherwise specified, the variables shown in the schemes below are as defined above in reference to Formula (I).

Compounds according to the invention may be synthesized by synthetic routes that include processes analogous to those well-known in the chemical arts, particularly in light of the description contained herein, and those for other heterocycles described in: Comprehensive Heterocyclic Chemistry II, Editors Katritzky and Rees, Elsevier, 1997, e.g. Volume 3; Liebigs Annalen der Chemie, (9):1910-16, (1985); Helvetica Chimica Acta, 41:1052-60, (1958); Arzneimittel-Forschung, 40(12):1328-31, (1990), each of which are expressly incorporated by reference. Starting materials are generally available from commercial sources such as Sigma-Aldrich Chemicals (Milwaukee, Wis.) or are readily prepared using methods well known to those skilled in the art (e.g., prepared by methods generally described in Louis F. Fieser and Mary Fieser, Reagents for Organic Synthesis, v. 1-23, Wiley, N.Y. (1967-2006 ed.), or Beilsteins Handbuch der organischen Chemie, 4, Aufl. ed. Springer-Verlag, Berlin, including supplements (also available via the Beilstein online database).

Synthetic chemistry transformations and protecting group methodologies (protection and deprotection) useful in synthesizing compounds according to the invention and necessary reagents and intermediates are known in the art and include, for example, those described in R. Larock, Comprehensive Organic Transformations, VCH Publishers (1989); T. W. Greene and P. G. M. Wuts, Protective Groups in Organic Synthesis, 3rd Ed., John Wiley and Sons (1999); and L. Paquette, ed., Encyclopedia of Reagents for Organic Synthesis, John Wiley and Sons (1995) and subsequent editions thereof. The need for such protection will vary depending on the nature of the remote functionality and the conditions of the preparation methods. Suitable amino-protecting groups include acetyl, trifluoroacetyl, t-butoxycarbonyl (BOC), benzyloxycarbonyl (CBz) and 9-fluorenylmethyleneoxycarbonyl (Fmoc). The need for such protection is readily determined by one skilled in the art.

Additional particularly useful reactions in preparing compounds of the present invention include alkylation, reductive amination, oxidation, reduction, and hydrolysis reactions. Such transformations are well within the ordinary skill in the art.

Compounds according to the invention may be prepared singly or as compound libraries comprising, for example, at least two, or 5 to 1,000 compounds, or 10 to 100 compounds. Libraries of compounds of the invention may be prepared by a combinatorial “split and mix” approach or by multiple parallel syntheses using either solution phase or solid phase chemistry, by procedures known to those skilled in the art. Thus, according to a further aspect of the invention there is provided a compound library comprising at least two compounds of the invention, or pharmaceutically acceptable salts thereof.

In the methods of preparing compounds according to the invention, it may be advantageous to separate reaction products from one another and/or from starting materials. The desired products of each step or series of steps is separated and/or purified to the desired degree of homogeneity by the techniques common in the art. Typically such separations involve multiphase extraction, crystallization from a solvent or solvent mixture, distillation, sublimation, or chromatography. Chromatography can involve any number of methods including, for example: reverse-phase and normal phase; size exclusion; ion exchange; high, medium and low pressure liquid chromatography methods and apparatus; small scale analytical; simulated moving bed (SMB) and preparative thin or thick layer chromatography, as well as techniques of small scale thin layer and flash chromatography.

Another class of separation methods involves treatment of a mixture with a reagent selected to bind to or render otherwise separable a desired product, unreacted starting material, reaction by product, or the like. Such reagents include adsorbents or absorbents such as activated carbon, molecular sieves, ion exchange media, or the like. Alternatively, the reagents can be acids in the case of a basic material, bases in the case of an acidic material, binding reagents such as antibodies, binding proteins, selective chelators such as crown ethers, liquid/liquid ion extraction reagents (LIX), or the like. Selection of appropriate methods of separation depends on the nature of the materials involved, such as, boiling point and molecular weight in distillation and sublimation, presence or absence of polar functional groups in chromatography, stability of materials in acidic and basic media in multiphase extraction, and the like.

A single stereoisomer, e.g., an enantiomer, substantially free of its stereoisomer may be obtained by resolution of the racemic mixture using a method such as formation of diastereomers using optically active resolving agents (Eliel, E. and Wilen, S. “Stereochemistry of Organic Compounds,” John Wiley & Sons, Inc., New York, 1994; Lochmuller, C. H., (1975) J. Chromatogr., 113(3):283-302). Racemic mixtures of chiral compounds of the invention can be separated and isolated by any suitable method, including: (1) formation of ionic, diastereomeric salts with chiral compounds and separation by fractional crystallization or other methods, (2) formation of diastereomeric compounds with chiral derivatizing reagents, separation of the diastereomers, and conversion to the pure stereoisomers, and (3) separation of the substantially pure or enriched stereoisomers directly under chiral conditions. See: “Drug Stereochemistry, Analytical Methods and Pharmacology,” Irving W. Wainer, Ed., Marcel Dekker, Inc., New York (1993).

Under method (1), diastereomeric salts can be formed by reaction of enantiomerically pure chiral bases such as brucine, quinine, ephedrine, strychnine, a-methyl-b-phenylethylamine (amphetamine), and the like with asymmetric compounds bearing acidic functionality, such as carboxylic acid and sulfonic acid. The diastereomeric salts may be induced to separate by fractional crystallization or ionic chromatography. For separation of the optical isomers of amino compounds, addition of chiral carboxylic or sulfonic acids, such as camphorsulfonic acid, tartaric acid, mandelic acid, or lactic acid can result in formation of the diastereomeric salts.

Alternatively, by method (2), the substrate to be resolved is reacted with one enantiomer of a chiral compound to form a diastereomeric pair (E. and Wilen, S. “Stereochemistry of Organic Compounds”, John Wiley & Sons, Inc., 1994, p. 322). Diastereomeric compounds can be formed by reacting asymmetric compounds with enantiomerically pure chiral derivatizing reagents, such as menthyl derivatives, followed by separation of the diastereomers and hydrolysis to yield the pure or enriched enantiomer. A method of determining optical purity involves making chiral esters, such as a menthyl ester, e.g., (−) menthyl chloroformate in the presence of base, or Mosher ester, a-methoxy-a-(trifluoromethyl)phenyl acetate of the racemic mixture and analyzing the 1H NMR spectrum for the presence of the two atropisomeric enantiomers or diastereomers (Jacob III. J. Org. Chem. (1982) 47:4165). Stable diastereomers of atropisomeric compounds can be separated and isolated by normal- and reverse-phase chromatography following methods for separation of atropisomeric naphthyl-isoquinolines (WO 96/15111). By method (3), a racemic mixture of two enantiomers can be separated by chromatography using a chiral stationary phase (“Chiral Liquid Chromatography” (1989) W. J. Lough, Ed., Chapman and Hall, New York; Okamoto, J. Chromatogr., (1990) 513:375-378). Enriched or purified enantiomers can be distinguished by methods used to distinguish other chiral molecules with asymmetric carbon atoms, such as optical rotation and circular dichroism.

### DETAILED DESCRIPTION OF EXPERIMENTS

Synthetic Method A: Fisher indole synthesis using beta substituted cyclic ketone and 2-carboxylate-phenyl hydrazine followed by esterification gave indole intermediate. Alkylation of indole nitrogen with the required alkyl bromide followed by hydrolysis gave rise to the desired product after purification.

### Representative Example: 7-butyl-5-[(3-carbamoylphenyl)methyl]-5H,6H,7H,8H,9H,10H-cyclohepta[b]indole-4-carboxylic acid

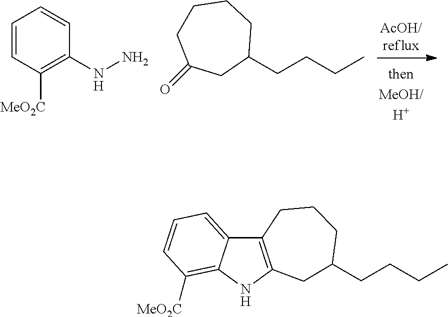 [SMILES: COC(=O)c1ccccc1NN ; CCCCC1CCCCC(=O)C1 ; CCCCC1CCCc2c([nH]c3c(C(=O)OC)cccc23)C1]

Step 1.1: Hydrazine (1.12 g) and ketone (3 g) was mixed in AcOH and stirred at 130° C. which after 3 hr, AcOH was distilled off. Reaction was then neutralized with saturated sodium bicarbonate and extracted with EtOAc (300 mL×3) which was dried and concentrated by rotary evaporation. Purification by column chromatography (30% EtOAc:Pet Ether) gave 1 g of desired indole product.
Step 1.2: 1 g of indole was dissolved in 15 mL of MeOH. 1 mL of H₂SO₄ was added and heated at 80° C. After 16 hrs, MeOH was distilled off from the reaction mixture, neutralized with saturated sodium bicarbonate and extracted with EtOAc (300 mL×3) which was dried and concentrated by rotary evaporation. Purification by column chromatography (20% EtOAc:Pet Ether) gave 900 mg of desired indole ester product

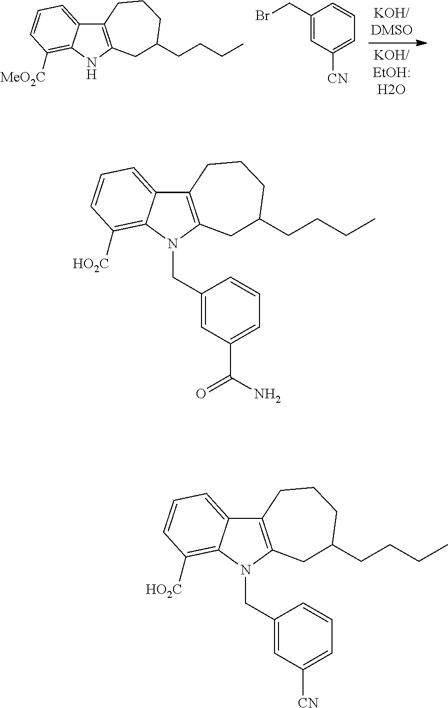

Step 2.1: The indole ester (900 mg) and the 3-Cyano-benzyl bromide (1.18 g) was dissolved in DMSO (10 mL) and then KOH (842 mg) was added at room temperature and stirred. After 2 hr, reaction was diluted with water and extracted with EtOAc (300 mL×3) which was dried and concentrated by rotary evaporation. Purification by column chromatography (15% EtOAc:Pet Ether) gave 700 mg of desired indole ester product
Step 2.2: Benzyl indole was dissolved in EtOH:H2O (30:6 mL) and then KOH (473 mg) was added at room temperature and heated to 70° C. After 15 min, reaction was cooled to r.t., neutralized with 1N HCl solution and extracted with EtOAc (300 mL×3). Collected organic extract was then dried and concentrated by rotary evaporation. Purification by MS directed purification gave 110 mg of 7-butyl-5-[(3-cyanophenyl)methyl]-5H,6H,7H,8H,9H,10H-cyclohepta[b]indole-4-carboxylic acid and 105 mg of 7-butyl-5-[(3-carbamoylphenyl)methyl]-5H,6H,7H,8H,9H,10H-cyclohepta[b]indole-4-carboxylic acid.
Synthetic Method B: Fisher indole synthesis using an unsubstituted cyclic ketone and 2-carboxylate-phenyl hydrazine followed by esterification gave indole intermediate. TFAA-DMSO alkylation protocol (Masanori Tayu et al., Org. Biomol. Chem. (2013) 1_496) with the required nucleophile followed by ester hydrolysis gave rise to the desired product after purification.

### Representative Example: 9-[(3-carbamoylphenyl)methyl]-2-pentyl-2,3,4,9-tetrahydro-1H-carbazole-8-carboxylic acid

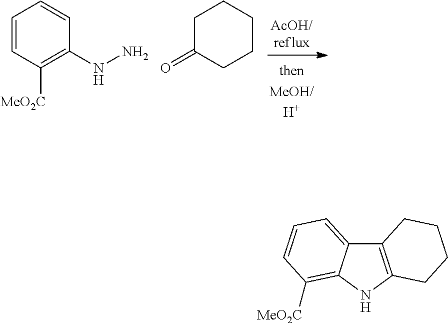

Step 1.1: Hydrazine (6.0 g) and ketone (6.2 g) was mixed in AcOH (100 mL) and stirred at 130° C. which after 3 hr, AcOH was distilled off. Reaction was then neutralized with saturated sodium bicarbonate and extracted with EtOAc (300 mL×3) which was dried and concentrated by rotary evaporation. Purification by column chromatography (30% EtOAc:Pet Ether) gave 5 g of desired indole product.
Step 1.2: 5 g of indole was dissolved in 100 mL of MeOH. 7 mL of concentrated H₂SO₄ was added and heated at 80° C. After 16 hours, MeOH was distilled off from the reaction mixture, neutralized with saturated sodium bicarbonate and extracted with EtOAc (300 mL×3) which was dried and concentrated by rotary evaporation. Purification by column chromatography (20% EtOAc:Pet Ether) gave 4.2 g of desired indole ester product.

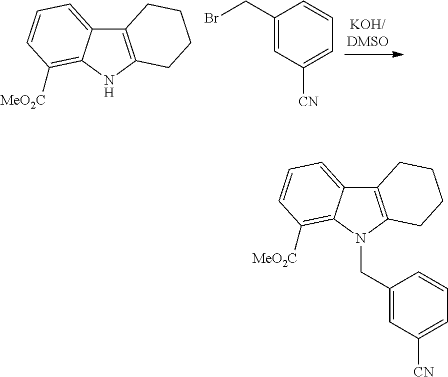

Step 2: The indole ester (3 g) was dissolved in DMSO (50 mL) and then KOH (3.675 g) was added at room temperature. 3-Cyano-benzyl bromide (5.13 g) was then added in portions and stirred. After 2 hr, reaction slowly poured into 1N HCl in flask with ice bath and then the organics were extracted with EtOAc (300 mL×3) which was dried and concentrated by rotary evaporation. Purification by column chromatography (20% EtOAc:Pet Ether) gave 3.5 g of desired indole ester product

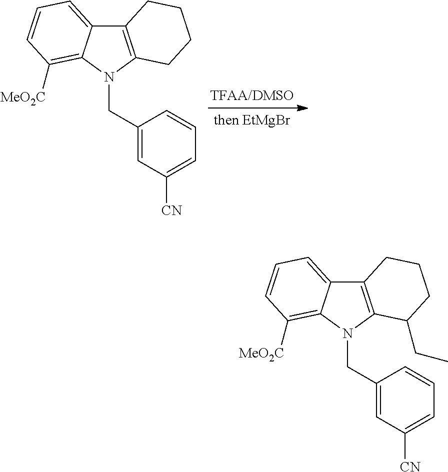 [SMILES: COC(=O)c1cccc2c3c(n(Cc4cccc(C#N)c4)c12)CCCC3 ; CCC1CCCc2c1n(Cc1cccc(C#N)c1)c1c(C(=O)OC)cccc21]

Step 3: To the solution of indole ester (500 mg) in dichloromethane (7 mL) at −40° C., was added DMSO (0.315 mL). to this mixture, Trifluoro-acetic anhydride (0.617 mL) was added dropwise and stirred at −40° C. After 1 hr, ethyl magnesium bromide (17.647 mL, 1M) was added dropwise to this mixture. After 2 hrs, the reaction was slowly poured into a solution of 10 mL of saturated NaHCO3+20 mL H2O+30 mL of EtOAc. Organic layer was separated and dried and concentrated in vacuo. Purification by column chromatography (20% EtOAc:Pet Ether) gave 300 mg of desired product

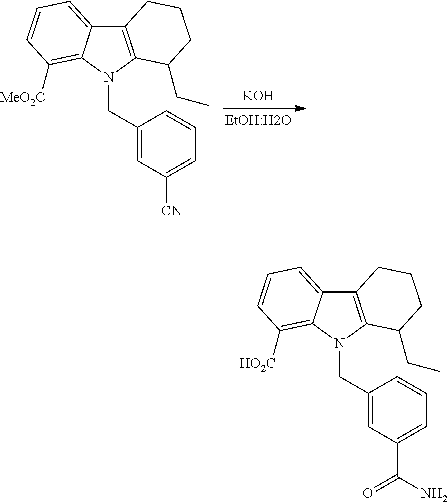

Step 4: Benzyl indole (150 mg) was dissolved in EtOH:H2O (5:2 mL) and then KOH (156 mg) was added at room temperature and stirred. After 16 hrs, reaction neutralized with 1N HCl solution and solid was filtered as 9-[(3-carbamoylphenyl)methyl]-2-pentyl-2,3,4,9-tetrahydro-1H-carbazole-8-carboxylic acid (30 mg).

### Antiviral Activity

Activity of compounds affecting viral replication and disease was tested against coronavirus replication in differentiated 3T3-L1 mouse adipocyte cells using human coronavirus OC43 as a model virus. 3T3-L1 preadipocytes (ATCC) were routinely cultured in a growth medium composed of DMEM high-glucose (Sigma), 10% FBS (Gibco), 10 U/ml penicillin and 10 μg/ml streptomycin (P/S; Gibco) in a standard cell culture incubator at 37° C. To induce adipogenic differentiation, a confluent layer of 3T3-L1 cells were incubated with the growth medium containing 2 μM rosiglitazone, 1 μM dexamethasone, 500 μM IBMX, and 1 μg/ml insulin (Sigma). Forty-eight (48) hours later (on day 2) and on days 4 and 6, medium of the cells was replaced with fresh medium containing 1 μg/ml insulin. On days 8 and 10, the medium was refreshed with regular growth medium and addition of insulin was omitted. On day 11 or 12, the cells were infected with human coronavirus OC43 (ATCC) at a multiplicity of 0.05 TCID50 (tissue-culture infectious-dose 50) per cell in DMEM containing 2% heat-inactivated FBS and P/S. One hour after infection, medium of the cells were replaced with fresh medium containing either the indicated FABP4 modulating compounds or the vehicle in which the compounds were dissolved (DMSO). The final compound concentration was 10 μM and final concentration of DMSO was 0.100 (v/v). The cells were incubated at 35° C. during the course of infection and compound treatment. Twenty-four hours after infection, medium of the cells was collected, cleared by centrifugation at 2,000 g for 5 minutes, and frozen at −80 degrees C. until further processing. The viral titers in the samples were determined by TCID50 assay in MRC5 cells (ATCC) using Reed-Munch method. and median fold reduction of virus titers in compound treated cells compared to vehicle control was calculated.

Table 1 shown below depicts the antiviral activity of the FABP4 modulating compounds. The compounds were categorized based on the magnitude of their antiviral activity. The compounds reduced virus titers >100-fold, between 10- to 99-fold, and between 2- to 9-fold compared to vehicle control were represented as “A”, “B” and “C”, respectively.

TABLE 1

| | | Antiviral |
|---|---|---|
| | | activity |
| Compound | IUPAC name | group |
| 1 | 7-butyl-5-[(3-carbamoylphenyl)methyl]- | B |
| | 5H,6H,7H,8H,9H,10H- | |
| | cyclohepta[b]indole-4-carboxylic acid | |
| 2 | 7-butyl-5-[(3-cyanophenyl)methyl]- | A |
| | 5H,6H,7H,8H,9H,10H- | |
| | cyclohepta[b]indole-4-carboxylic acid | |
| 3 | 7-butyl-5-[(4-methylphenyl)methyl]- | B |
| | 5H,6H,7H,8H,9H,10H- | |
| | cyclohepta[b]indole-4-carboxylic acid | |
| 4 | 7-butyl-5-[(2-methylphenyl)methyl]- | A |
| | 5H,6H,7H,8H,9H,10H- | |
| | cyclohepta[b]indole-4-carboxylic acid | |
| 5 | 5-[(3-carbamoylphenyl)methyl]-7-hexyl- | B |
| | 5H,6H,7H,8H,9H,10H- | |
| | cyclohepta[b]indole-4-carboxylic acid | |
| 6 | 5-[(3-cyanophenyl)methyl]-7-hexyl- | A |
| | 5H,6H,7H,8H,9H,10H- | |
| | cyclohepta[b]indole-4-carboxylic acid | |
| 7 | 9-[(3-carbamoylphenyl)methyl]-2-hexyl- | B |
| | 2,3,4,9-tetrahydro-1H-carbazole-8- | |
| | carboxylic acid | |
| 8 | 9-[(3-cyanophenyl)methyl]-2-hexyl- | B |
| | 2,3,4,9-tetrahydro-1H-carbazole-8- | |
| | carboxylic acid | |
| 9 | 2-hexyl-9-[(pyridin-3-yl)methyl]-2,3,4,9- | A |
| | tetrahydro-1H-carbazole-8-carboxylic | |
| | acid | |
| 10 | 2-hexyl-9-[(2-methylphenyl)methyl]- | A |
| | 2,3,4,9-tetrahydro-1H-carbazole-8- | |
| | carboxylic acid | |
| 11 | 9-[(2-fluorophenyl)methyl]-2-hexyl- | A |
| | 2,3,4,9-tetrahydro-1H-carbazole-8- | |
| | carboxylic acid | |
| 12 | 1-butyl-9-[(3-carbamoylphenyl)methyl]- | C |
| | 2,3,4,9-tetrahydro-1H-carbazole-8- | |
| | carboxylic acid | |
| 13 | 1-butyl-9-[(3-cyanophenyl)methyl]- | B |
| | 2,3,4,9-tetrahydro-1H-carbazole-8- | |
| | carboxylic acid | |
| 14 | 6-butyl-5-[(3-carbamoylphenyl)methyl]- | B |
| | 5H,6H,7H,8H,9H,10H- | |
| | cyclohepta[b]indole-4-carboxylic acid | |
| 15 | 6-butyl-5-[(3-cyanophenyl)methyl]- | B |
| | 5H,6H,7H,8H,9H, 10H- | |
| | cyclohepta[b]indole-4-carboxylic acid | |
| 16 | 3-butyl-4-[(3-carbamoylphenyl)methyl]- | C |
| | 1H,2H,3H,4H-cyclopenta[b]indole-5- | |
| | carboxylic acid | |
| 17 | 2-butyl-4-[(3-carbamoylphenyl)methyl]- | C |
| | 1H,2H,3H,4H-cyclopenta[b]indole-5- | |
| | carboxylic acid | |
| 18 | 2-butyl-4-[(3-cyanophenyl)methyl]- | C |
| | 1H,2H,3H,4H-cyclopenta[b]indole-5- | |
| | carboxylic acid | |
| 19 | 10-butyl-5-[(3-carbamoylphenyl)methyl]- | C |
| | 5H,6H,7H,8H,9H,10H- | |
| | cyclohepta[b]indole-4-carboxylic acid | |
| 20 | 3-butyl-9-[(3-carbamoylphenyl)methyl]- | C |
| | 2,3,4,9-tetrahydro-1H-carbazole-8- | |
| | carboxylic acid | |
| 21 | 2-((2′-(5-Ethyl-3,4-diphenyl-1H-pyrazol- | C |
| | 1-yl)-[1,1′-biphenyl]-3-yl)oxy)acetic acid | |
| 22 | 6-chloro-4-phenyl-2-(piperidin-1-yl)-3- | A |
| | (1H-tetrazol-5-yl)quinoline | |
| 23 | 5-((3-chloro-2-methylphenoxy)methyl)- | B |
| | 2-phenyl-[1,2,4]triazolo[1,5-a]pyrimidin- | |
| | 7(4H)-one | |
| 24 | 3-(5-cyclopropyl-3-(3,5-dimethyl-1H- | B |
| | pyrazol-4-yl)-2-(3-isopropoxyphenyl)- | |
| | 1H-indol-1-yl)propanoic acid | |

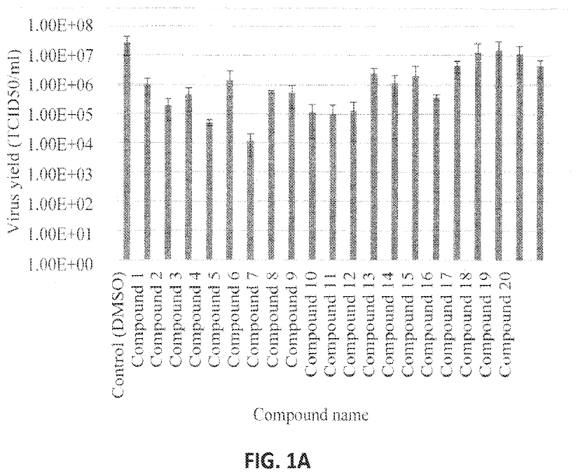

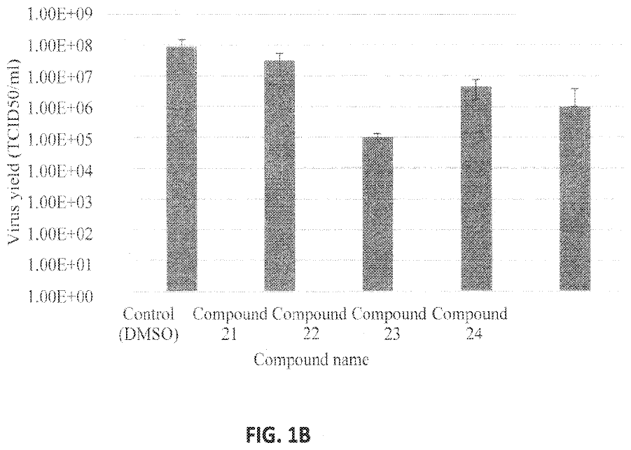

FIG. 1A and FIG. 1B depict graphical charts showing the virus yield obtained from cells treated with the corresponding compounds and vehicle control (DMSO). More specifically, FIG. 1 includes a graph for the vehicle control (DMSO) and different FABP4 modulating compounds and a second graph for the FABP4 modulating compounds 2-((2′-(5-Ethyl-3,4-diphenyl-1H-pyrazol-1-yl)-[1,1′-biphenyl]-3-yl)oxy)acetic acid (Compound 21), referred to as BMS309403 in the art, 6-chloro-4-phenyl-2-(piperidin-1-yl)-3-(1H-tetrazol-5-yl)quinoline (Compound 22), 5-((3-chloro-2-methylphenoxy)methyl)-2-phenyl-[1,2,4]triazolo[1,5-a]pyrimidin-7(4H)-one (Compound 23), 3-(5-cyclopropyl-3-(3,5-dimethyl-1H-pyrazol-4-yl)-2-(3-isopropoxyphenyl)-1H-indol-1-yl)propanoic acid (Compound 24) and their corresponding vehicle control. Each of the graphs include an x-axis associated with compound name and a y-axis associated with a viral titer in TCID50/ml. Data are shown as mean (±standard deviation) of two or three biological replicates.

According to one embodiment of the invention, compounds of the invention demonstrate antiviral activity against coronavirus. The subject matter of the present invention is particularly advantageous due to its unexpected results with coronavirus. For example, as described herein, compounds of the invention is particularly efficacious in treating coronavirus.

Although this invention has been described with a certain degree of particularity, it is to be understood that the present disclosure has been made only by way of illustration and that numerous changes in the details of construction and arrangement of parts may be resorted to without departing from the spirit and the scope of the invention.

What is claimed is:

1. A method for treating a viral disease in a subject comprising administering to said subject a therapeutically effective dose of one or more of the compounds described in Formula (I): 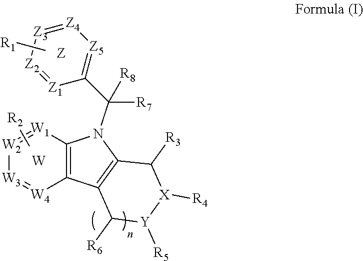
Wherein:
W₁₋₄ and Z₁-Z₅ are each independently —C, —CH, CH₂, O, S, or N;
X is independently CH₂, N or CHR₄;
Y is independently CH₂, or CHR₅;
n is a number between 0 and 3;
One or more R₁'s on the ring Z are independently selected from the group consisting of: CN, OH, COOH, OCH₃, CF₃, CONH₂, B(OH)₂, B(OR)₂, an acid isostere, a substituted amine, ethers, and a halogen, substituted or unsubstituted alkyl, substituted or unsubstituted cyclic or heterocyclic, substituted or unsubstituted cycloaryl or cychloheteroaryl, wherein the substituted cycloaryl or cychloheteroaryl may be substituted with hydrogen, CN, OH, COOH, OCH₃, CF₃, CONH₂, B(OH)₂, B(OR)₂, an acid isostere, a substituted amine, ethers, and a halogen, substituted or unsubstituted alkyl, substituted or unsubstituted cyclic or heterocyclic, substituted or unsubstituted cycloaryl or cychloheteroaryl, and SO₂NH₂;
One or more R₂'s on the ring W are independently selected from the group consisting of: CN, OH, CHF2, CH2F, CF₃, COOH, CONH₂, B(OH)₂, B(OR)₂, an acid isostere, a halogen, and a bicyclic heteroaryl;
R₇ is hydrogen or CN, COOH, CONH₂, B(OH)₂, B(OR)₂ or an acid isostere;
R is alkyl;
when n is not zero, R₃, R₄, R₅ or R₈, or R₆ is each independently selected from:
(1) hydrogen;
(2) substituted or unsubstituted alkyl or ether having 1 to 12 carbon atoms,
(3) a substituted amine, or
(4) —(CH₂)ₘ G, wherein m is 1 to 12 and G is independently selected from:
(a) cycloalkyl containing 3 to 6 carbon atoms,
(b) aryl or heteroaryl,
(c) CF₃, CF₂H or CFH₂, or
(d) a heterocycle,
provided that R₃, R₄, R₅, R₈, or R₆ are not all hydrogen;
or pharmaceutically acceptable salts or stereoisomers thereof.

2. The method according to claim 1, wherein R₁ and R₂ are both present and each is independently CN, COOH, or CONH₂.

3. The method according to claim 1, wherein the Formula I includes more than one R₁ and more than one R₂.

4. The method according to claim 1, wherein R₃, R₄, R₅ R₈, or R₆ when n is not zero, is each independently alkyl having 4 carbon atoms.

5. The method according to claim 1, wherein R₃, R₄, R₅ R₈, or R₆ when n is not zero, is each independently alkyl having 5 carbon atoms.

6. The method according to claim 1, wherein R₃, R₄, R₅ R₈, or R₆ when n is not zero, is each independently alkyl having 6 carbon atoms.

7. The method according to claim 1, wherein the ring Z contains Z₁-Z₄.

8. The method according to claim 1, wherein the ring Z contains Z₁-Z₅.

9. The method according to claim 1, wherein the R₁ on the ring Z is the halogen.

10. The method according to claim 1, wherein the R₁ on the ring Z is the CN.

11. The method according to claim 1, wherein the R₁ on the ring Z is the CF₃.

12. The method according to claim 1, wherein the R₂ on the ring W is the halogen.

13. The method according to claim 1, wherein the R₁ on the ring Z comprise the CN and/or the halogen.

14. The method according to claim 1, wherein the R₁ on the ring Z comprise the CN and/or the halogen, and wherein the R₂ on the ring W comprise another halogen.

15. The method according to claim 14, wherein the halogen is identical to the other halogen.

16. The method according to claim 14, wherein the halogen differs from the other halogen.

17. The method according to claim 1, wherein each of the one or more compounds are a pure optical isomer.

18. The method according to claim 1 for use in the prophylaxis or treatment of viral disorders by acting on the fatty acid binding protein FABP4.

19. The method according to claim 18, wherein the viral disorders are selected from common cold, SARS, and COVID-19.

20. The method according to claim 1, wherein the one or more compounds are delivered as pharmaceutical compositions.

21. A method for treating a viral disease in a subject comprising administering to said subject a therapeutically effective dose of one or more compounds described in Formula (II): 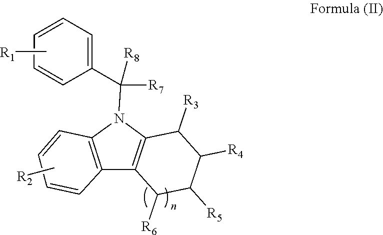
Wherein:
n=0, 1, or 2;
R₁ is selected from the group consisting of: CN, COOH, CONH₂, B(OH)₂, B(OR)₂, an acid isostere, and a halogen;
R₂ is selected from the group consisting of: CN, COOH, CONH₂, B(OH)₂, B(OR)₂, an acid isostere, a halogen, and a bicyclic compound;
R₇ is hydrogen or CN, COOH, CONH₂, B(OH)₂, B(OR)₂ or an acid isostere;
R is alkyl;
R₃, R₄, R₅ or R₈, or R₆ when n is not zero, is each independently selected from:
(1) hydrogen;
(2) alkyl having 1 to 12 carbon atoms, or
(3) —(CH₂)ₘG, wherein m is 1 to 12 and G is independently selected from:
(a) cycloalkyl containing 3 to 6 carbon atoms,
(b) aryl or heteroaryl, or
(c) CF₃, CF₂H or CFH₂;
provided that G is not a nitrogen or oxygen-containing group; and
provided that R₃, R₄, R₅, R₈, or R₆ are not all hydrogen;
or pharmaceutically acceptable salts thereof.

22. The method according to claim 21, wherein R₁ and R₂ are both present, and each is independently CN, COOH, or CONH₂.

23. The method according to claim 21, wherein the Formula II includes more than one R₁ and more than one R₂.

24. The method according to claim 21, wherein R₃, R₄, R₅, R₈, or R₆ when n is not zero, is each independently alkyl having 4 carbon atoms.

25. The method according to claim 21, wherein R₃, R₄, R₅, R₈, or R₆ when n is not zero, is each independently alkyl having 5 carbon atoms.

26. The method according to claim 21, wherein R₃, R₄, R₅, R₈, or R₆ when n is not zero, is each independently alkyl having 6 carbon atoms.

27. The method according to claim 21, wherein each of the one or more compounds are a pure optical isomer.

28. The method according to claim 21 for use in the prophylaxis or treatment of viral disorders by acting on the fatty acid binding protein FABP4.

29. The method according to claim 28, wherein the viral disorders are selected from common cold, SARS, and COVID-19.

30. The method according to claim 21, wherein the one or more compounds are delivered as pharmaceutical compositions.

31. A method for treating a viral disease in a subject comprising administering to said subject a therapeutically effective dose of one or more of the compounds described in Formula (III): 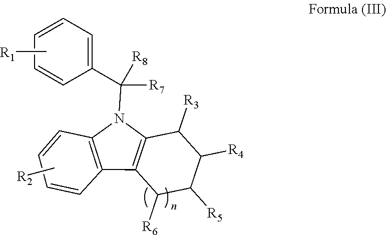
Wherein:
n=0, 1, or 2;
R₁ and R₂ are each independently halogen, alkyl, cycloalkyl, aryl, heteroaryl, CN, COOH, CONH₂, B(OH)₂, B(OR)₂, or an acid isostere;
R₇ is hydrogen, alkyl, cycloalkyl, aryl, heteroaryl, CN, COOH, CONH₂, B(OH)₂, B(OR)₂ or an acid isostere;
R is alkyl;
R₃, R₄, R₅ or R₈, or R₆ when n is not zero, are each independently selected from:
(1) hydrogen;
(2) alkyl having 1 to 12 carbon atoms, or
(3) —(CH₂)ₘG, wherein m is 1 to 12 and G is independently selected from:
(a) cycloalkyl containing 3 to 6 carbon atoms;
(b) aryl or heteroaryl; or
(c) CF₃, CF₂H, or CFH₂;
provided that R₃, R₄, R₅, R₈, or R₆ are not all hydrogen;
or pharmaceutically acceptable salts or stereoisomers thereof.

32. The method according to claim 31, wherein R₃, R₄, R₅, R₈, or R₆ when n is 1, is each independently alkyl having 4 carbon atoms.

33. The method according to claim 31, wherein R₃, R₄, R₅, R₈, or R₆ when n is 1, is each independently alkyl having 5 carbon atoms.

34. The method according to claim 31, wherein R₃, R₄, R₅, R₈, or R₆ when n is 1, is each independently alkyl having 6 carbon atoms.

35. The method according to claim 31, wherein each of the one or more compounds are a pure optical isomer.

36. The method according to claim 31 for use in the prophylaxis or treatment of viral disorders by acting on the fatty acid binding protein FABP4.

37. The method according to claim 36, wherein the viral disorders are selected from common cold, SARS, and COVID-19.

38. The method according to claim 31, wherein the one or more compounds are delivered as pharmaceutical compositions.

39. A method for treating a viral disease in a subject comprising administering to said subject a therapeutically effective dose of one or more of the of compounds described in Formula (IV): 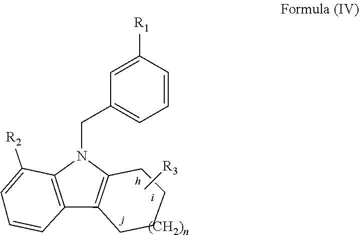
Wherein:
n=0, 1 or 2;
R₁ and R₂ are each independently CN, COOH or CONH₂;
R₃ is independently selected from:
(1) alkyl having 1 to 12 carbon atoms;
(2) —(CH₂)ₘG, wherein m is 1 to 12 and G is independently selected from:
(a) cycloalkyl containing 3 to 6 carbon atoms;
(b) phenyl; and
provided that G is not a nitrogen or oxygen-containing group;
or pharmaceutically acceptable salts thereof.

40. The method according to claim 39, wherein n=0, R₃ is attached to the h-, i- or j-position.

41. The method according to claim 39, wherein: n=1 and R₃ is attached to the h-, i- or j-position.

42. The method according to claim 39, wherein: n=2 and R₃ is attached to the h-, i- or j-position.

43. The method according to claim 39, wherein each of the one or more compounds are a pure optical isomer.

44. The method according to claim 39 for use in the prophylaxis or treatment of viral disorders by acting on the fatty acid binding protein FABP4.

45. The method according to claim 44, wherein the viral disorders are selected from common cold, SARS, and COVID-19.

46. The method according to claim 39, wherein the one or more compounds are delivered as pharmaceutical compositions.

47. A method for treating a viral disease in a subject comprising administering to said subject a therapeutically effective dose of one or more of the compounds selected from the group consisting of:
5-[(3-cyanophenyl)methyl]-2-fluoro-7-hexyl-5H,6H,7H,8H,9H,10H-cyclohepta[b]indole-4-carboxylic acid, 5-[(6-cyanopyridin-2-yl)methyl]-7-hexyl-5H,6H,7H,8H,9H,10H-cyclohepta[b]indole-4-carboxylic acid, 5-[(6-carbamoylpyridin-2-yl)methyl]-7-hexyl-5H,6H,7H,8H,9H,10H-cyclohepta[b]indole-4-carboxylic acid, 6-({4-carboxy-7-hexyl-5H,6H,7H,8H,9H,10H-cyclohepta[b]indol-5-yl}methyl)pyridine-2-carboxylic acid, 5-[(3-cyano-2-fluorophenyl)methyl]-7-hexyl-5H,6H,7H,8H,9H,10H-cyclohepta[b]indole-4-carboxylic acid, 5-[(1,3-benzoxazol-6-yl)methyl]-7-hexyl-5H,6H,7H,8H,9H,10H-cyclohepta[b]indole-4-carboxylic acid, 5-[(1,3-benzoxazol-5-yl)methyl]-7-hexyl-5H,6H,7H,8H,9H,10H-cyclohepta[b]indole-4-carboxylic acid, 5-[(6-fluoropyridin-2-yl)methyl]-7-hexyl-5H,6H,7H,8H,9H,10H-cyclohepta[b]indole-4-carboxylic acid, 5-[(2-fluoropyridin-4-yl)methyl]-7-hexyl-5H,6H,7H,8H,9H,10H-cyclohepta[b]indole-4-carboxylic acid, 7-hexyl-5-{[6-(trifluoromethyl)pyridin-2-yl]methyl}-5H,6H,7H,8H,9H,10H-cyclohepta[b]indole-4-carboxylic acid, 7-hexyl-5-{[2-(trifluoromethyl)pyridin-4-yl]methyl}-5H,6H,7H,8H,9H,10H-cyclohepta[b]indole-4-carboxylic acid, 5-[(5-cyanopyridin-3-yl)methyl]-7-hexyl-5H,6H,7H,8H,9H,10H-cyclohepta[b]indole-4-carboxylic acid, 5-[(5-cyanothiophen-2-yl)methyl]-7-hexyl-5H,6H,7H,8H,9H,10H-cyclohepta[b]indole-4-carboxylic acid, 5-[(4-cyanothiophen-2-yl)methyl]-7-hexyl-5H,6H,7H,8H,9H,10H-cyclohepta[b]indole-4-carboxylic acid, 5-[(5-cyanofuran-2-yl)methyl]-7-hexyl-5H,6H,7H,8H,9H,10H-cyclohepta[b]indole-4-carboxylic acid, 5-[(3,5-dimethyl-1,2-oxazol-4-yl)methyl]-7-hexyl-5H,6H,7H,8H,9H,10H-cyclohepta[b]indole-4-carboxylic acid, 5-(3-cyanobenzoyl)-7-hexyl-5H,6H,7H,8H,9H,10H-cyclohepta[b]indole-4-carboxylic acid, 5-[(1,3-benzoxazol-7-yl)methyl]-7-hexyl-5H,6H,7H,8H,9H,10H-cyclohepta[b]indole-4-carboxylic acid, 5-[(5-cyanothiophen-3-yl)methyl]-7-hexyl-5H,6H,7H,8H,9H,10H-cyclohepta[b]indole-4-carboxylic acid, 7-hexyl-5-[(1H-indol-4-yl)methyl]-5H,6H,7H,8H,9H,10H-cyclohepta[b]indole-4-carboxylic acid, 5-[(3-carbamoylphenyl)methyl]-7-propyl-5H,6H,7H,8H,9H,10H-cyclohepta[b]indole-4-carboxylic acid, 7-butyl-5-[(3-carbamoylphenyl)methyl]-5H,6H,7H,8H,9H,10H-cyclohepta[b]indole-4-carboxylic acid, 7-butyl-5-[(3-cyanophenyl)methyl]-5H,6H,7H,8H,9H,10H-cyclohepta[b]indole-4-carboxylic acid, 7-butyl-5-[(pyridin-3-yl)methyl]-5H,6H,7H,8H,9H,10H-cyclohepta[b]indole-4-carboxylic acid, 7-butyl-5-[(3-methylphenyl)methyl]-5H,6H,7H,8H,9H,10H-cyclohepta[b]indole-4-carboxylic acid, 7-butyl-5-[(3-methoxyphenyl)methyl]-5H,6H,7H,8H,9H,10H-cyclohepta[b]indole-4-carboxylic acid, 7-butyl-5-[(3-chlorophenyl)methyl]-5H,6H,7H,8H,9H,10H-cyclohepta[b]indole-4-carboxylic acid, 7-butyl-5-[(3-hydroxyphenyl)methyl]-5H,6H,7H,8H,9H,10H-cyclohepta[b]indole-4-carboxylic acid, 7-butyl-5-[(2-methoxypyridin-4-yl)methyl]-5H,6H,7H,8H,9H,10H-cyclohepta[b]indole-4-carboxylic acid, 7-butyl-5-[(4-carbamoylphenyl)methyl]-5H,6H,7H,8H,9H,10H-cyclohepta[b]indole-4-carboxylic acid, 7-butyl-5-[(2-carbamoylphenyl)methyl]-5H,6H,7H,8H,9H,10H-cyclohepta[b]indole-4-carboxylic acid, 7-butyl-5-[(4-methylphenyl)methyl]-5H,6H,7H,8H,9H,10H-cyclohepta[b]indole-4-carboxylic acid, 7-butyl-5-[(2-cyanophenyl)methyl]-5H,6H,7H,8H,9H,10H-cyclohepta[b]indole-4-carboxylic acid, 7-butyl-5-[(2-methylphenyl)methyl]-5H,6H,7H,8H,9H,10H-cyclohepta[b]indole-4-carboxylic acid, 7-butyl-5-[(2-fluorophenyl)methyl]-5H,6H,7H,8H,9H,10H-cyclohepta[b]indole-4-carboxylic acid, 5-[(3-carbamoylphenyl)methyl]-7-pentyl-5H,6H,7H,8H,9H,10H-cyclohepta[b]indole-4-carboxylic acid, 5-[(3-cyanophenyl)methyl]-7-pentyl-5H,6H,7H,8H,9H,10H-cyclohepta[b]indole-4-carboxylic acid, 5-[(3-carbamoylphenyl)methyl]-7-(2-phenylethyl)-5H,6H,7H,8H,9H,10H-cyclohepta[b]indole-4-carboxylic acid, 5-[(3-cyanophenyl)methyl]-7-(2-phenylethyl)-5H,6H,7H,8H,9H,10H-cyclohepta[b]indole-4-carboxylic acid, 5-[(3-carbamoylphenyl)methyl]-7-hexyl-5H,6H,7H,8H,9H,10H-cyclohepta[b]indole-4-carboxylic acid, 5-[(3-cyanophenyl)methyl]-7-hexyl-5H,6H,7H,8H,9H,10H-cyclohepta[b]indole-4-carboxylic acid, 5-[(3-carbamoylphenyl)methyl]-7-octyl-5H,6H,7H,8H,9H,10H-cyclohepta[b]indole-4-carboxylic acid, 5-[(3-cyanophenyl)methyl]-7-octyl-5H,6H,7H,8H,9H,10H-cyclohepta[b]indole-4-carboxylic acid, 5-[(3-fluorophenyl)methyl]-7-hexyl-5H,6H,7H,8H,9H,10H-cyclohepta[b]indole-4-carboxylic acid, 7-hexyl-5-[(pyridin-3-yl)methyl]-5H,6H,7H,8H,9H,10H-cyclohepta[b]indole-4-carboxylic acid, 7-hexyl-5-[(3-methylphenyl)methyl]-5H,6H,7H,8H,9H,10H-cyclohepta[b]indole-4-carboxylic acid, 7-hexyl-5-[(3-methoxyphenyl)methyl]-5H,6H,7H,8H,9H,10H-cyclohepta[b]indole-4-carboxylic acid, 5-[(3-chlorophenyl)methyl]-7-hexyl-5H,6H,7H,8H,9H,10H-cyclohepta[b]indole-4-carboxylic acid, 7-hexyl-5-[(2-methoxypyridin-4-yl)methyl]-5H,6H,7H,8H,9H,10H-cyclohepta[b]indole-4-carboxylic acid, 5-[(3-carboxyphenyl)methyl]-7-hexyl-5H,6H,7H,8H,9H,10H-cyclohepta[b]indole-4-carboxylic acid, 5-[(4-carbamoylphenyl)methyl]-7-hexyl-5H,6H,7H,8H,9H,10H-cyclohepta[b]indole-4-carboxylic acid, 5-[(2-carbamoylphenyl)methyl]-7-hexyl-5H,6H,7H,8H,9H,10H-cyclohepta[b]indole-4-carboxylic acid, 7-hexyl-5-[(4-methylphenyl)methyl]-5H,6H,7H,8H,9H,10H-cyclohepta[b]indole-4-carboxylic acid, 5-[(4-cyanophenyl)methyl]-7-hexyl-5H,6H,7H,8H,9H,10H-cyclohepta[b]indole-4-carboxylic acid, 5-[(2-cyanophenyl)methyl]-7-hexyl-5H,6H,7H,8H,9H,10H-cyclohepta[b]indole-4-carboxylic acid, 7-hexyl-5-[(2-methylphenyl)methyl]-5H,6H,7H,8H,9H,10H-cyclohepta[b]indole-4-carboxylic acid, 5-[(2-fluorophenyl)methyl]-7-hexyl-5H,6H,7H,8H,9H,10H-cyclohepta[b]indole-4-carboxylic acid, 5-[(4-fluorophenyl)methyl]-7-hexyl-5H,6H,7H,8H,9H,10H-cyclohepta[b]indole-4-carboxylic acid, 9-[(3-carbamoylphenyl)methyl]-2-hexyl-2,3,4,9-tetrahydro-1H-carbazole-8-carboxylic acid, 9-[(3-cyanophenyl)methyl]-2-hexyl-2,3,4,9-tetrahydro-1H-carbazole-8-carboxylic acid, 9-[(3-carboxyphenyl)methyl]-2-hexyl-2,3,4,9-tetrahydro-1H-carbazole-8-carboxylic acid, 9-[(3-fluorophenyl)methyl]-2-hexyl-2,3,4,9-tetrahydro-1H-carbazole-8-carboxylic acid, 2-hexyl-9-[(pyridin-3-yl)methyl]-2,3,4,9-tetrahydro-1H-carbazole-8-carboxylic acid, 2-hexyl-9-[(3-methylphenyl)methyl]-2,3,4,9-tetrahydro-1H-carbazole-8-carboxylic acid, 2-hexyl-9-[(3-methoxyphenyl)methyl]-2,3,4,9-tetrahydro-1H-carbazole-8-carboxylic acid, 9-[(3-chlorophenyl)methyl]-2-hexyl-2,3,4,9-tetrahydro-1H-carbazole-8-carboxylic acid, 2-hexyl-9-[(3-hydroxyphenyl)methyl]-2,3,4,9-tetrahydro-1H-carbazole-8-carboxylic acid, 2-hexyl-9-[(2-methoxypyridin-4-yl)methyl]-2,3,4,9-tetrahydro-1H-carbazole-8-carboxylic acid, 9-[(3-carboxyphenyl)methyl]-2-hexyl-2,3,4,9-tetrahydro-1H-carbazole-8-carboxylic acid, 9-[(4-carbamoylphenyl)methyl]-2-hexyl-2,3,4,9-tetrahydro-1H-carbazole-8-carboxylic acid, 9-[(2-carbamoylphenyl)methyl]-2-hexyl-2,3,4,9-tetrahydro-1H-carbazole-8-carboxylic acid, 2-hexyl-9-[(4-methylphenyl)methyl]-2,3,4,9-tetrahydro-1H-carbazole-8-carboxylic acid, 9-[(4-cyanophenyl)methyl]-2-hexyl-2,3,4,9-tetrahydro-1H-carbazole-8-carboxylic acid, 9-[(2-cyanophenyl)methyl]-2-hexyl-2,3,4,9-tetrahydro-1H-carbazole-8-carboxylic acid, 2-hexyl-9-[(2-methylphenyl)methyl]-2,3,4,9-tetrahydro-1H-carbazole-8-carboxylic acid, 9-[(2-fluorophenyl)methyl]-2-hexyl-2,3,4,9-tetrahydro-1H-carbazole-8-carboxylic acid, 9-[(3-carbamoylphenyl)methyl]-2-(2-phenylethyl)-2,3,4,9-tetrahydro-1H-carbazole-8-carboxylic acid, 9-[(3-cyanophenyl)methyl]-2-(2-phenylethyl)-2,3,4,9-tetrahydro-1H-carbazole-8-carboxylic acid, 9-[(3-carbamoylphenyl)methyl]-4-(2-phenylethyl)-2,3,4,9-tetrahydro-1H-carbazole-8-carboxylic acid, 9-[(3-cyanophenyl)methyl]-4-(2-phenylethyl)-2,3,4,9-tetrahydro-1H-carbazole-8-carboxylic acid, 9-[(3-carbamoylphenyl)methyl]-2-propyl-2,3,4,9-tetrahydro-1H-carbazole-8-carboxylic acid, 9-[(3-cyanophenyl)methyl]-2-propyl-2,3,4,9-tetrahydro-1H-carbazole-8-carboxylic acid, 2-butyl-9-[(3-carbamoylphenyl)methyl]-2,3,4,9-tetrahydro-1H-carbazole-8-carboxylic acid, 9-[(3-carbamoylphenyl)methyl]-2-pentyl-2,3,4,9-tetrahydro-1H-carbazole-8-carboxylic acid, 9-[(3-cyanophenyl)methyl]-2-pentyl-2,3,4,9-tetrahydro-1H-carbazole-8-carboxylic acid, 1-butyl-9-[(3-carbamoylphenyl)methyl]-2,3,4,9-tetrahydro-1H-carbazole-8-carboxylic acid, 9-[(3-carbamoylphenyl)methyl]-1-(pentyloxy)-2,3,4,9-tetrahydro-1H-carbazole-8-carboxylic acid, 9-[(3-cyanophenyl)methyl]-1-(pentyloxy)-2,3,4,9-tetrahydro-1H-carbazole-8-carboxylic acid, 1-butyl-9-[(3-cyanophenyl)methyl]-2,3,4,9-tetrahydro-1H-carbazole-8-carboxylic acid, 6-butyl-5-[(3-carbamoylphenyl)methyl]-5H,6H,7H,8H,9H,10H-cyclohepta[b]indole-4-carboxylic acid, 6-butyl-5-[(3-cyanophenyl)methyl]-5H,6H,7H,8H,9H,10H-cyclohepta[b]indole-4-carboxylic acid, 9-[(3-carbamoylphenyl)methyl]-1-ethyl-2,3,4,9-tetrahydro-1H-carbazole-8-carboxylic acid, 9-[(3-cyanophenyl)methyl]-1-ethyl-2,3,4,9-tetrahydro-1H-carbazole-8-carboxylic acid, 9-[(3-carboxyphenyl)methyl]-1-ethyl-2,3,4,9-tetrahydro-1H-carbazole-8-carboxylic acid, 9-[(3-carbamoylphenyl)methyl]-1-propoxy-2,3,4,9-tetrahydro-1H-carbazole-8-carboxylic acid, 9-[(3-carbamoylphenyl)methyl]-4-ethyl-2,3,4,9-tetrahydro-1H-carbazole-8-carboxylic acid, 4-[(3-carbamoylphenyl)methyl]-3-ethyl-1H,2H,3H,4H-cyclopenta[b]indole-5-carboxylic acid, 4-[(3-cyanophenyl)methyl]-3-ethyl-1H,2H,3H,4H-cyclopenta[b]indole-5-carboxylic acid, 3-butyl-4-[(3-carbamoylphenyl)methyl]-1H,2H,3H,4H-cyclopenta[b]indole-5-carboxylic acid, 3-butyl-4-[(3-cyanophenyl)methyl]-1H,2H,3H,4H-cyclopenta[b]indole-5-carboxylic acid, 2-butyl-4-[(3-carbamoylphenyl)methyl]-1H,2H,3H,4H-cyclopenta[b]indole-5-carboxylic acid, 2-butyl-4-[(3-cyanophenyl)methyl]-1H,2H,3H,4H-cyclopenta[b]indole-5-carboxylic acid, 5-[(3-carbamoylphenyl)methyl]-10-ethyl-5H,6H,7H,8H,9H,10H-cyclohepta[b]indole-4-carboxylic acid, 5-[(3-cyanophenyl)methyl]-10-ethyl-5H,6H,7H,8H,9H,10H-cyclohepta[b]indole-4-carboxylic acid, 5-[(3-carbamoylphenyl)methyl]-10-propyl-5H,6H,7H,8H,9H,10H-cyclohepta[b]indole-4-carboxylic acid, 5-[(3-cyanophenyl)methyl]-10-propyl-5H,6H,7H,8H,9H,10H-cyclohepta[b]indole-4-carboxylic acid, 9-[(3-carboxyphenyl)methyl]-4-ethyl-2,3,4,9-tetrahydro-1H-carbazole-8-carboxylic acid, 9-[(3-carbamoylphenyl)methyl]-3-ethyl-2,3,4,9-tetrahydro-1H-carbazole-8-carboxylic acid, 10-butyl-5-[(3-carbamoylphenyl)methyl]-5H,6H,7H,8H,9H,10H-cyclohepta[b]indole-4-carboxylic acid, 10-butyl-5-[(3-cyanophenyl)methyl]-5H,6H,7H,8H,9H,10H-cyclohepta[b]indole-4-carboxylic acid, 5-[(3-carbamoylphenyl)methyl]-10-pentyl-5H,6H,7H,8H,9H,10H-cyclohepta[b]indole-4-carboxylic acid, 5-[(3-cyanophenyl)methyl]-10-pentyl-5H,6H,7H,8H,9H,10H-cyclohepta[b]indole-4-carboxylic acid, 4-[(3-carbamoylphenyl)methyl]-2-pentyl-1H,2H,3H,4H-cyclopenta[b]indole-5-carboxylic acid, 4-[(3-cyanophenyl)methyl]-2-pentyl-1H,2H,3H,4H-cyclopenta[b]indole-5-carboxylic acid, 9-[(3-carbamoylphenyl)methyl]-2-ethyl-2,3,4,9-tetrahydro-1H-carbazole-8-carboxylic acid, 9-[(3-carboxyphenyl)methyl]-2-ethyl-2,3,4,9-tetrahydro-1H-carbazole-8-carboxylic acid, 5-[(3-carbamoylphenyl)methyl]-7-ethyl-5H,6H,7H,8H,9H,10H-cyclohepta[b]indole-4-carboxylic acid, 5-[(3-cyanophenyl)methyl]-7-ethyl-5H,6H,7H,8H,9H,10H-cyclohepta[b]indole-4-carboxylic acid, 9-[(3-cyanophenyl)methyl]-3-ethyl-2,3,4,9-tetrahydro-1H-carbazole-8-carboxylic acid, 9-[(3-carbamoylphenyl)methyl]-4-propyl-2,3,4,9-tetrahydro-1H-carbazole-8-carboxylic acid, 9-[(3-carbamoylphenyl)methyl]-3-propyl-2,3,4,9-tetrahydro-1H-carbazole-8-carboxylic acid, 9-[(3-cyanophenyl)methyl]-3-propyl-2,3,4,9-tetrahydro-1H-carbazole-8-carboxylic acid, 4-butyl-9-[(3-carbamoylphenyl)methyl]-2,3,4,9-tetrahydro-1H-carbazole-8-carboxylic acid, 4-butyl-9-[(3-cyanophenyl)methyl]-2,3,4,9-tetrahydro-1H-carbazole-8-carboxylic acid, 3-butyl-9-[(3-carbamoylphenyl)methyl]-2,3,4,9-tetrahydro-1H-carbazole-8-carboxylic acid, 3-butyl-9-[(3-cyanophenyl)methyl]-2,3,4,9-tetrahydro-1H-carbazole-8-carboxylic acid, 9-[(3-carbamoylphenyl)methyl]-4-pentyl-2,3,4,9-tetrahydro-1H-carbazole-8-carboxylic acid, 9-[(3-cyanophenyl)methyl]-4-pentyl-2,3,4,9-tetrahydro-1H-carbazole-8-carboxylic acid, 9-[(3-carbamoylphenyl)methyl]-3-pentyl-2,3,4,9-tetrahydro-1H-carbazole-8-carboxylic acid, 9-[(3-cyanophenyl)methyl]-3-pentyl-2,3,4,9-tetrahydro-1H-carbazole-8-carboxylic acid, 4-[(3-carbamoylphenyl)methyl]-3-propyl-1H,2H,3H,4H-cyclopenta[b]indole-5-carboxylic acid, 4-[(3-cyanophenyl)methyl]-3-propyl-1H,2H,3H,4H-cyclopenta[b]indole-5-carboxylic acid, 2-({7-butyl-5-[(3-carbamoylphenyl)methyl]-5H,6H,7H,8H,9H,10H-cyclohepta[b]indol-4-yl}formamido)acetic acid, 2-({7-butyl-5-[(3-cyanophenyl)methyl]-5H,6H,7H,8H,9H,10H-cyclohepta[b]indol-4-yl}formamido)acetic acid, 7-butyl-5-[(3-carbamoylphenyl)methyl]-N-(2-hydroxyethyl)-5H,6H,7H,8H,9H,10H-cyclohepta[b]indole-4-carboxamide, 7-butyl-5-[(3-cyanophenyl)methyl]-N-(2-hydroxyethyl)-5H,6H,7H,8H,9H,10H-cyclohepta[b]indole-4-carboxamide, 7-butyl-5-[(3-fluorophenyl)methyl]-5H,6H,7H,8H,9H,10H-cyclohepta[b]indole-4-carboxylic acid, and 7-butyl-5-[(3-carboxyphenyl)methyl]-5H,6H,7H,8H,9H,10H-cyclohepta[b]indole-4-carboxylic acid, or pharmaceutically acceptable salts or stereoisomers thereof.

48. A method for treating a viral disease in a subject comprising administering to said subject a therapeutically effective dose of one or more of the compounds selected from the group consisting of: 2-((2′-(5-Ethyl-3,4-diphenyl-1H-pyrazol-1-yl)-[1,1′-biphenyl]-3-yl)oxy)acetic acid, 6-chloro-4-phenyl-2-(piperidin-1-yl)-3-(1H-tetrazol-5-yl)quinoline, 5-((3-chloro-2-methylphenoxy)methyl)-2-phenyl-[1,2,4]triazolo[1,5-a]pyrimidin-7(4H)-one, and 3-(5-cyclopropyl-3-(3,5-dimethyl-1H-pyrazol-4-yl)-2-(3-isopropoxyphenyl)-1H-indol-1-yl)propanoic acid, or pharmaceutically acceptable salts or stereoisomers thereof.

49. The method according to claim 47, wherein each of the one or more compounds are a pure optical isomer.

50. The method according to claim 47 for use in the prophylaxis or treatment of viral disorders by acting on the fatty acid binding protein FABP4.

51. The method according to claim 50, wherein the viral disorders are selected from common cold, SARS, and COVID-19.

52. The method according to claim 47, wherein the one or more compounds are delivered as pharmaceutical compositions.

53. A method for treating a viral disease in a subject comprising administering to said subject a therapeutically effective dose of one or more of the compounds described in Formula (V): 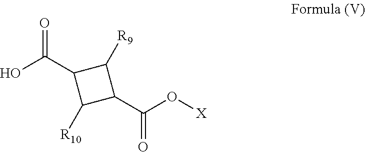
Wherein:
R₉ and R₁₀ are each independently a phenyl or anisole; and
X comprises naphthalene or fluorene;
or pharmaceutically acceptable salts or stereoisomers thereof.

54. The method of claim 53, wherein said subject is a human subject.

55. A method for treating a viral disease in a subject comprising administering to said subject a therapeutically effective dose of one or more of the compounds described in Formula (VI): 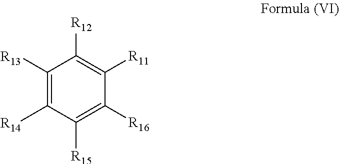
Wherein:
R₁₁ is —C(CH₃)₂ or 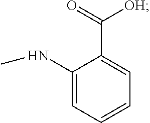
R₁₂ is —H, —O, a halogen, a phenyl, or piperidine;
R₁₃ is —C(CH₃)₂, a halogen, or 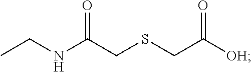
R₁₄ is a halogen, —CF₃, —NHCH₃, coumaran or 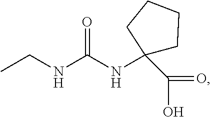
R₁₅ is —C(CH₃)₂ or a phenyl; and
R₁₆ is —COOH, a halogen, —SO₃H, 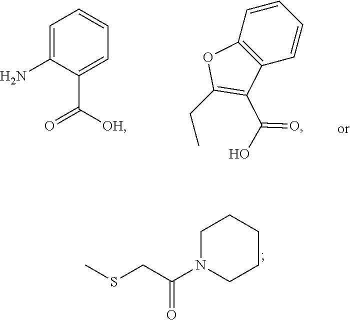
or pharmaceutically acceptable salts or stereoisomers thereof.

56. The method of claim 55, wherein said subject is a human subject.

57. A method for treating a viral disease in a subject comprising administering to said subject a therapeutically effective dose of one or more of the compounds described in Formula (VII): 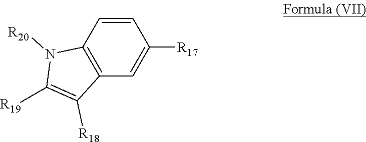
Wherein:
R₁₇ is cycloalkane;
R₁₈ is 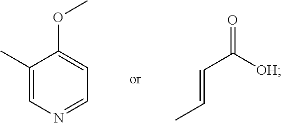
R₁₉ is a phenyl; and
R₂₀ is —(CH₂)₂COOH or 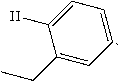
wherein H is a halogen;
or pharmaceutically acceptable salts or stereoisomers thereof.

58. The method of claim 57, wherein said subject is a human subject.

59. A method for treating a viral disease in a subject comprising administering to said subject a therapeutically effective dose of one or more of the compounds described in Formula (VIII): 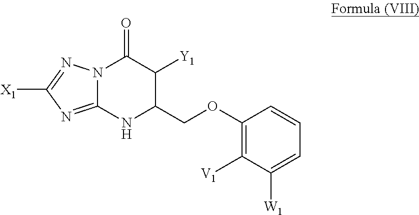
Wherein:
X₁ is a phenyl;
Y₁ and W₁ are independently a halogen; and
V₁ is —CH₃ or cycloalkane;
or pharmaceutically acceptable salts or stereoisomers thereof.

60. The method of claim 59, wherein said subject is a human subject.

61. A method for treating a viral disease in a subject comprising administering to said subject a therapeutically effective dose of one or more of the compounds described in Formula (IX): 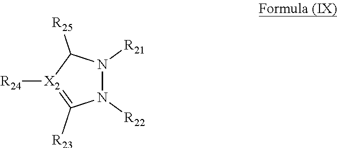
Wherein:
X₂ is —CH or O;
R₂₁ is —H or 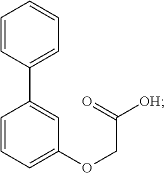 [SMILES: O=C(O)COc1cccc(-c2ccccc2)c1]
R₂₂ is —H, —O, a phenyl, or 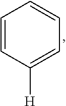
wherein H is a halogen;
R₂₃ is —H, thiophene, a phenyl, 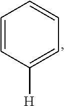
wherein H is a halogen; and
R₂₄ is —H or a phenyl; and
R₂₅ is —CH₂CH₃, 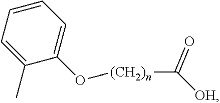
or 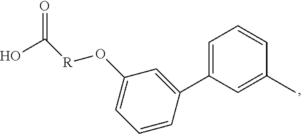
wherein n is 3 or 4, and wherein R is —CH₂ or —C(CH₃)₂;
or pharmaceutically acceptable salts or stereoisomers thereof.

62. The method of claim 61, wherein said subject is a human subject.

63. A method for treating a viral disease in a subject comprising administering to said subject a therapeutically effective dose of a compound selected from the group consisting of:
2-((2′-(5-ethyl-3,4-diphenyl-1H-pyrazol-1-yl)-[1,1′-biphenyl]-3-yl)oxy)acetic acid, 2-((2-oxo-2-((2-(piperidin-1-yl)benzyl)amino)ethyl)thio)acetic acid, (3,5-dibromo-4-hydroxyphenyl)(2-ethylbenzofuran-3-yl)methanone, (E)-4-((5-(methoxycarbonyl)-[2,2′-bithiophen]-3-yl)amino)-4-oxobut-2-enoic acid, 2-(1-(methoxymethyl)cyclopentyl)-6-pentyl-4-phenyl-3-(2H-tetrazol-5-yl)-5,6,7,8-tetrahydroquinoline, 2-cyclohexyl-4-(2-methylpyridin-4-yl)-3-(2H-tetrazol-5-yl)-6-(trifluoromethyl)-5,6,7,8-tetrahydroquinoline, 6-chloro-4-phenyl-2-(piperidin-1-yl)-3-(2H-tetrazol-5-yl)quinoline, 6-chloro-2-methyl-4-phenylquinoline-3-carboxylic acid, N-(2-(4-(1-allyl-2,4-dioxo-2,3,4,5-tetrahydro-1H-pyrrolo[3,2-d]pyrimidin-6-yl)phenoxy)ethyl)picolinamide, (5-chloro-3-hydroxyquinoxalin-2-yl)(2-(3,5-dichlorobenzyl)pyrrolidin-1-yl)methanone, 1-(3-(4,6-dichloro-[1,1′-biphenyl]-2-yl)ureido)cyclopentane-1-carboxylic acid, 5-((3-chloro-2-methylphenoxy)methyl)-2-phenyl-[1,2,4]triazolo[1,5-a]pyrimidin-7(4H)-one, 6-chloro-5-((3-chloro-2-cyclopropylphenoxy)methyl)-2-phenyl-[1,2,4]triazolo[1,5-a]pyrimidin-7(4H)-one, 2-((3-chloro-2-(2,3-dihydrobenzofuran-5-yl)phenyl)amino)benzoic acid, 2-((6-chloro-5-(methylamino)-[1,1′-biphenyl]-2-yl)amino)benzoic acid, (1S,2R,3S,4R)-2,4-bis(2-methoxyphenyl)-3-((naphthalen-1-yloxy)carbonyl)cyclobutane-1-carboxylic acid, (1R,2R,3R,4R)-3-(((9H-fluoren-9-yl)methoxy)carbonyl)-2,4-bis(2-methoxyphenyl)cyclobutane-1-carboxylic acid, (1R,2R,3R,4R)-3-((naphthalen-1-yloxy)carbonyl)-2,4-diphenylcyclobutane-1-carboxylic acid, 1-[1-[4,4-Bis(4-fluorophenyl)butyl]-4-piperidinyl]-1,3-dihydro-2H-benzimidazole-2-one, 2,4,6-triisopropylbenzoic acid, 2,4,6-triisopropylbenzenesulfonic acid, 2-((2′-(1-(4-chlorophenyl)-5-(thiophen-2-yl)-1H-pyrazol-3-yl)-[1,1′-biphenyl]-3-yl)oxy)-2-methylpropanoic acid, (E)-3-(1-(2-fluorobenzyl)-1H-indol-3-yl)acrylic acid, 4-(2-(5-(2-chlorophenyl)-1-phenyl-1H-pyrazol-3-yl)phenoxy)butanoic acid, 5-(2-(4,5-diphenyloxazol-2-yl)phenoxy)pentanoic acid, 2-((2′-(4,5-diphenyloxazol-2-yl)-[1,1′-biphenyl]-3-yl)oxy)acetic acid, 4-(9H-carbazol-9-yl)butanoic acid, 3-((9H-carbazol-9-yl)sulfonyl)thiophene-2-carboxylic acid, 5-(3-carbamoylbenzyl)-5,6,7,8,9,10-hexahydrocyclohepta[b]indole-4-carboxylic acid, 9-(2-(trifluoromethyl)benzyl)-2,3,4,9-tetrahydro-1H-carbazole-8-carboxylic acid, 3-(5-cyclopropyl-3-(3,5-dimethyl-1H-pyrazol-4-yl)-2-(3-isopropoxyphenyl)-1H-indol-1-yl)propanoic acid, 3-(5-cyclopropyl-3-(3-methoxypyridin-4-yl)-2-phenyl-1H-indol-1-yl)propanoic acid, and 2-((2-oxo-2-(piperidin-1-yl)ethyl)thio)-6-(trifluoromethyl)pyrimidin-4(3H)-one.

64. A method for treating a viral disease in a subject comprising administering to said subject a therapeutically effective dose of one or more of the compounds described in Formula X: 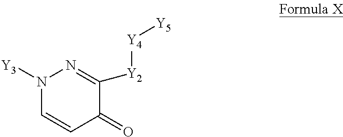
Wherein:
Y₂ is CR¹³R¹³, O, NR¹⁵ or S;
Y₃ is heteroaryl or aryl, wherein said heteroaryl and aryl groups are optionally substituted with one to three groups independently selected from the group consisting of halo, cyano, C₁₋₆ alkyl, (C₁₋₆ alkyl)R¹⁷, OR¹⁷, (C=0)NR¹⁵R¹⁶, heterocyclyl, aryl, heteroaryl and (heteroaryl)R¹⁶;
Y₄ is CR¹⁴R¹⁴0, C₂₋₃ alkenyl, O, NR¹⁵ or S;
Y₅ is heteroaryl or phenyl, wherein said heteroaryl and phenyl groups are optionally substituted with one to three groups independently selected from the group consisting of halo, cyano, oxo, C₁₋₆ alkyl, (C₁₋₆ alkyl)0R¹⁶, 0R¹⁶, R¹⁷, 0R¹⁷, 0(C₁₋₆ alkyl)0R¹⁶, 0(C₁₋₆ alkyl)R¹⁷, (C=0)R¹⁶, (C=0)0R¹⁶, (C=0)NHR¹⁶, (C=0)R¹⁷, NHR¹⁶, NH(C0)0R¹⁶, NH(C=0)R¹⁷, NH(C=0)0, (C₁₋₆ alkyl)0R¹⁶ and N0₂;
R¹³ is hydrogen, halo or C₁₋₆ alkyl;
R¹⁴ is hydrogen, halo, C₁₋₆ alkyl or (C=0)0R¹⁶;
R¹⁵ is hydrogen or C₁₋₆ alkyl,
R¹⁶ is hydrogen or C₁₋₆ alkyl, wherein said alkyl is optionally substituted with one to three groups independently selected from the group consisting of halo and hydroxyl;
R¹⁷ is hydrogen, heterocyclyl, aryl or heteroaryl, wherein said heterocyclyl and heteroaryl groups are optionally substituted with one to two groups independently selected from the group consisting of cyano, halo, hydroxyl, R¹⁶, R¹⁸, 0R¹⁶, (C₁₋₆ alkyl)0R¹⁶, (C₁₋₆ alkyl)0R¹⁸, S0iC₁₋₆ alkyl), (C=0)R¹⁸; and
R¹⁸ is heterocyclyl or heteroaryl, wherein said heterocyclyl group is optionally substituted with cyano, halo, hydroxyl, R¹⁶, 0R¹⁶ or (C=0)0R¹⁶;
or a pharmaceutically acceptable salt thereof.

65. A method for treating a viral disease in a subject comprising administering to said subject a therapeutically effective dose of one or more of the compounds described in Formula (XI): 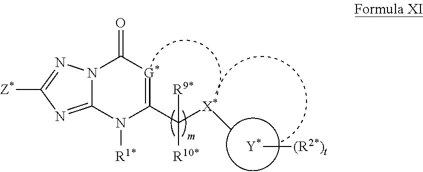
wherein:
Y* is a ring as described below;
X* is either:
(i) C(R>7′oR8^{α}), N(FT), S, S(O₂), or O, and both 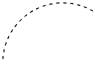
are absent; or
(ii) C(R⁷), and X* is linked, either to the ring marked Y* as shown by the 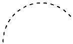
towards the right of X*, or to the position marked G* on the triazolopyrimidinone ring as shown by the 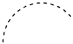
towards the left of X*, to form a 3-8 membered cycloalkyl or a 4-8 membered heterocyclyl ring containing 1-3 heteroatoms independently selected from N, O and S, or (iii) N (with R³ being absent), and X* is linked, either to the ring marked Y* as shown by the 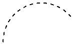
towards the right of X*, or to the position marked G* on the triazolopyrimidinone ring as shown by the 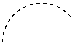
towards the left of X*, to form a 4-8 membered heterocyclyl ring containing 1-3 heteroatoms independently selected from N, O and S, including the nitrogen atom of X*; further wherein 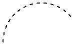
shown to the right of X* is mutually exclusive of 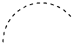
shown to the left of X*;
G* is C(R⁶) when 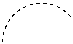
shown to the left of X* is absent, and G* is C when 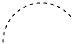
shown to the left of X* is present;
Ring Y* is aryl, heteroaryl, heterocyclyl or cycloalkyl, and when 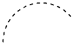
shown to the right of X* is present, a ring atom on Y* is the point of attachment for said 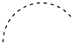
shown to the right of X*, wherein each of said aryl, heteroaryl, heterocyclyl and cycloalkyl can be unsubstituted or optionally independently substituted with one or more substituents which can be the same or different, each substituent being independently selected from the group consisting of halo, hydroxyl, alkyl, alkoxy, CN and CF₃;
R₃ may be present or absent as stated above, and when R³ is present, R³ is H, alkyl or cycloalkyl, wherein each of said alkyl and cycloalkyl may be unsubstituted or optionally independently substituted with one or more substituents which can be the same or different, each substituent being independently selected from the group consisting of halo, hydroxyl, alkoxy, CN and CF₃;
R¹* is H, alkyl, -alkyl-OR⁴, haloalkyl, haloalkoxy or -alkyl-CN; m is 1-2; t is 0-4; each
R²* is independently selected from the group consisting of halo, CN, —OR⁵, alkyl, haloalkyl, haloalkoxy, aryl, heteroaryl, heterocyclyl, cycloalkyl, —NH₂, —NH(alkyl), —NH(aryl), —NH(heteroaryl), —NH(cycloalkyl), —S-alkyl, —S-aryl, —S-heteroaryl, —S-cycloalkyl, —C(O)OH, —C(O)—NH₂, —C(O)N(H)(alkyl), —C(O)N(H)aryl, —C(O)N(H)(heteroaryl), —C(O)N(H)(heterocyclyl), —C(O)—N(H)(cycloalkyl), —C(O)N(alkyl)₂, —C(O)N(aryl)₂, —C(O)N(heteroaryl)₂, —C(O)N(heterocyclyl)₂, —C(O)—N(cycloalkyl)₂, —C(O)N(aryl)(alkyl), —C(O)N(heteroaryl)(aryl), —C(O)N(heterocyclyl)(heteroaryl), —C(O)N(aryl)(heterocyclyl), —C(O)—N(alkyl)(cycloalkyl), —C(O)N(cycloalkyl)(aryl), —C(O)N(cycloalkyl)(heterocyclyl), —NH—C(O)—NH₂, —C(O)R⁵, and —C(O)OR⁵, wherein each of said alkyl, aryl, heteroaryl, heterocyclyl and cycloalkyl may be unsubstituted or optionally independently substituted with one or more substituents which can be the same or different, each substituent being independently selected from the group consisting of halo, alkyl, aryl, heteroaryl, hydroxyl, cycloalkyl, alkoxy, —C(O)R⁵, CN and CF₃;
R* is H, —OR⁵, —C(O)OR⁵, alkyl, alkenyl, alkynyl, cycloalkyl, haloalkyl, aryl or heteroaryl, wherein each of said alkyl, cycloalkyl, haloalkyl, aryl and heteroaryl may be unsubstituted or optionally independently substituted with one or more substituents which can be the same or different, each substituent being independently selected from the group consisting of halo, CN, —OR⁵, alkyl, haloalkyl, haloalkoxy, aryl, heteroaryl, heterocyclyl, cycloalkyl, —C(O)—NH₂, —C(O)N(H)(CH₃), —NH—C(O)—NH₂, —C(O)R⁵, and —C(O)OR⁵;
R⁴ is H, alkyl, aryl or heteroaryl, wherein each of said alkyl, aryl and heteroaryl may be unsubstituted or optionally independently substituted with one or more substituents which can be the same or different, each substituents being independently selected from the group consisting of halo, hydroxyl, alkoxy, CN and CF₃;
R⁵ is H, alkyl, cycloalkyl, aryl or heteroaryl, wherein each of said alkyl, cycloalkyl, aryl and heteroaryl may be unsubstituted or optionally independently substituted with one or more substituents which can be the same or different, each substituents being independently selected from the group consisting of halo, hydroxyl, alkoxy, —OC(O)R⁴, CN and CF₃; R⁶ is H, halo, alkyl, aryl or heteroaryl, wherein each of said alkyl, aryl and heteroaryl may be unsubstituted or optionally independently substituted with one or more substituents which can be the same or different, each substituents being independently selected from the group consisting of halo, hydroxyl, alkoxy, CN and CF₃; R⁷ is H, alkyi, hydroxy or alkoxy, wherein each of said alkyl and alkoxy may be independently unsubstituted or optionally independently substituted with one or more substituents which can be the same or different, each substituent being independently selected from the group consisting of halo, hydroxyl, alkoxy, CN and CF₃;
R⁸ is H, alkyl, hydroxy or alkoxy, wherein each of said alkyl and alkoxy may be independently unsubstituted or optionally independently substituted with one or more substituents which can be the same or different, each substituent being independently selected from the group consisting of halo, hydroxyl, alkoxy, CN and CF₃;
R⁹* is H, halo, alkyl, cycloalkyl, aryl or heteroaryl, wherein each of said alkyl, aryl and heteroaryl may be unsubstituted or optionally independently substituted with one or more substituents which can be the same or different, each substituents being independently selected from the group consisting of halo, hydroxyl, alkoxy, CN and CF₃; and
R¹⁰* is H, halo, alkyl, cycloalkyl, aryl or heteroaryl, —C(O)OH, —C(O)—NH₂, —C(O)N(H)(alkyl), —C(O)N(H)aryl, —C(O)N(H)(heteroaryl), —C(O)N(H)(heterocyclyl), —C(O)—N(H)(cycloalkyl), —C(O)N(alkyl)₂, —C(O)N(aryl)₂, —C(O)N(heteroaryl)₂, —C(O)N(heterocyclyl)₂, —C(O)—N(cycloalkyl)₂, —C(O)N(aryl)(alkyl), —C(O)N(heteroaryl)(aryl), —C(O)N(heterocyclyl)(heteroaryl), —C(O)N(aryl)(heterocyclyl), —C(O)—N(alkyl)(cycloalkyl), —C(O)N(cycloalkyl)(aryl), —C(O)N(cycloalkyl)(heterocyclyl), —NH—C(O)—NH₂, —C(O)R⁵, and —C(O)OR⁵, wherein each of said alkyl, aryl, heteroaryl, heterocyclyl and cycloalkyl may be unsubstituted or optionally independently substituted with one or more substituents which can be the same or different, each substituent being independently selected from the group consisting of halo, alkyl, aryl, heteroaryl, hydroxyl, cycloalkyl, alkoxy, —C(O)R⁵, CN and CF₃; or alternatively,
R⁹* and R¹⁰* can together form ═O or R⁹* and R¹⁰* can be joined to form a spirocyclyl group.

66. A method for treating a viral disease in a subject comprising administering to said subject a therapeutically effective dose of one or more of the compounds described in Formula (XII): 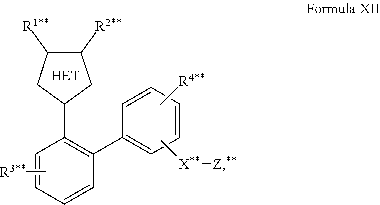
its stereoisomers, a pharmaceutically acceptable salt or prodrug ester thereof,
wherein R¹** and R²** are the same or different and are independently selected from, alkyl, cycloalkyl, cycloalkenyl, aryl, aralkyl, or;
R³** is selected from hydrogen, halogen, alkyl, alkenyl, alkynyl, alkoxy, cycloalkyl, cycloalkylalkyl, cycloalkenyl, alkylcarbonyl, cycloalkenylalkyl, haloalkyl, polyhaloalkyl, cyano, nitro, hydroxy, amino, alkanoyl, alkylthio, alkylsulfonyl, alkoxycarbonyl, alkylaminocarbonyl, alkylcarbonylamino, alkylcarbonyloxy, alkylaminosulfonyl, alkylamino, dialkylamino, all optionally substituted through available carbon atoms with 1, 2, 3, 4 or 5 groups selected from hydrogen, halo, alkyl, polyhaloalkyl, alkoxy, haloalkoxy, polyhaloalkoxy, alkoxycarbonyl, alkenyl, alkynyl, cycloalkyl, cycloalkylalkyl, hydroxy, hydroxyalkyl, nitro, cyano, amino, substituted amino, akylamino, dialkylamino, thiol, alkylthio, alkylcarbonyl, acyl, alkoxycarbonyl, aminocarbonyl, alkynylaminocarbonyl, alkylaminocarbonyl, alkenylaminocarbonyl, alkylcarbonyloxy, alkylcarbonylamino, arylcarbonylamino, alkoxycarbonylamino, alkylsulfonyl, aminosulfinyl, aniinosulfonyl, alkylsulfinyl, sulfonamido or sulfonyl;
R⁴** is selected from hydrogen, halogen, alkyl, alkenyl, alkynyl, alkoxy, aryl, arylalkyl, arylalkenyl, arylalkynyl, cycloalkyl, cycloalkylalkyl, polycycloalkyl, polycycloalkylaikyl, cycloalkenyl, cycloalkynyl, alkylcarbonyl, arylcarbonyl, cycloheteroalkyl, cycloheteroalkylalkyl, cycloalkenylalkyl, polycycloalkenyl, polycycloalkenylalkyl, polycycloalkynyl, polycycloalkynylalkyl, haloalkyl, polyhaloalkyl, cyano, nitro, hydroxy, amino, alkanoyl, aroyl, alkylsulfonyl, arylsulfonyl, alkoxycarbonyl, aryloxycarbonyl, alkylaminocarbonyl, arylaminocarbonyl, alkylcarbonylamino, alkoxycarbonyloxy, alkylaminosulfonyl, arylaminosulfonyl, alkylamino, dialkylamino, all optionally substituted through available carbon atoms with 1, 2, 3, 4 or 5 groups selected from hydrogen, halo, haloalkyl, polyhaloalkyl, alkoxy, haloalkoxy, polyhaloalkoxy, alkoxycarbonyl, alkenyl, alkynyl, cycloalkyl, cycloalkylalkyl, aryl, arylalkyl, arylcycloalkyl, arylalkenyl, arylalkynyl, aryloxy, aryloxyalkyl, arylalkoxy, arylazo, hydroxy, hydroxyalkyl, nitro, cyano, amino, substituted amino, alkylamino, dialkylamino, thiol, alkylthio, arylthio, arylthioalkyl, alkylcarbonyl, arylcarbonyl, acyl, arylaminocarbonyl, alkoxycarbonyl, aminocarbonyl, alkynylaminocarbonyl, alkylaminocarbonyl, alkenylaminocarbonyl, alkylcarbonyloxy, arylcarbonyloxy, alkylcarbonylamino, arylcarbonylamino, alkoxycarbonylamino, arylsulfinyl, arylsulfmylalkyl, arylsulfonyl, alkylsulfonyl, aminosulfinyl, aminosulfonyl, arylsulfonylamino, alkylsulfinyl, sulfonamido or sulfonyl;
X* is a bond or a linker group selected from (CH₂)ₙ, O(CH₂)ₙ, S(CH₂)ₙ, cycloalkylene, N(R⁵**)(CH₂)ₙ, NHCO, or CH═CH where n=0-5 and R⁵ is hydrogen, alkyl, or alkanoyl;
Z** is CO₂H or tetrazole of the formula 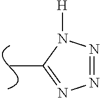
or its tautomer; and the group 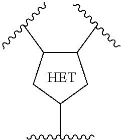 [SMILES: *C1CC(*)C(*)C1]
represents a heteroaryl group of the formula 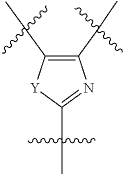
where Y is NR⁸** or O;
which may further be optionally substituted with one or two groups, which may be the same or different and are independently selected from alkyl, alkenyl, hydroxyalkyl, keto, carboxyalkyl, carboxy, cycloalkyl, alkoxy, formyl, alkanoyl, alkoxyalkyl or alkoxycarbonyl,
with the provisos that
(1) n≠o when Z** is CO₂H and X** is O(CH₂)ₙ, S(CH₂)ₙ or N(R⁵)(CH₂)ₙ), and
(2) when 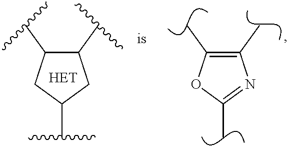
then X**—Z** may not be O-lower alkylene-CO₂H or —O-lower alkylene-CO₂alkyl when R¹** and R²** are both aryl or substituted aryl and R²** and R⁴** are each hydrogen.
